(19) United States
(12) Patent Application Publication
(10) Pub. No.: US 2004/0204341 A1
(43) Pub. Date: Oct. 14, 2004

(54) Nr2b receptor antagonists for the treatment or prevention of migraines

(76) Inventors: Christopher Allen, Doylestown, PA (US); Ken S. Koblan, Carversville, PA (US); Timothy Sleeth, Blue Bell, PA (US)

Correspondence Address: MERCK AND CO INC, P O BOX 2000, RAHWAY, NJ, 070650907

(21) Appl. No.: 10/479,923

(22) PCT Filed: Jun. 7, 2002
(86) PCT No.: PCT/US02/21069

Related U.S. Application Data
(60) Provisional application No. 60/297,672, filed on Jun. 12, 2001.

(51) Int. Cl. A61K 31/415; A61K 38/00; A61K 31/365; A61K 31/18

(52) U.S. Cl. 514/2; 514/406; 514/471; 514/602

(57) ABSTRACT

The present invention encompasses a method for treating or preventing migraines in a mammalian patient in need of such treatment or prevention comprising administering to said patient an NR2B receptor antagonist in an amount that is effective to treat or prevent migraines. The invention also encompasses the combination of an NR2B antagonist with a cyclooxygenase-2 selective inhibitor, a calcitonin gene-related peptide receptor (CGRP) ligand, a leukotriene receptor antagonist or a 5HT_{1B/1D} agonist for the treatment or prevention of migraines.

### BACKGROUND OF THE INVENTION

Migraines are recurrent, often familial, symptom complexes of periodic attacks of vascular headache. The condition is characterized by intermittent attacks of headache, preceded by an aura in approximately 15% of patients. The headache is often accompanied by associated symptoms, most commonly nausea, vomiting, photophobia and phonophobia. Migraines affect approximately 17% of adult women and 6% of adult men (Stewart et al., Neurology, 1994, 44 (suppl. 4), 517-523). This invention relates to a method for treating or preventing migraines comprising administering an NR2B receptor antagonist.

Ions such as glutamate play a key role in processes related to chronic pain and pain-associated neurotoxicity—primarily by acting through N-methyl-D-aspartate (“NMDA”) receptors. Thus, inhibition of such action—by employing ion channel antagonists, particularly NMDA antagonists—can be beneficial in the treatment and control of pain.

Known NMDA antagonists include ketamine, dextrometorphan, and 3-(2-carboxypiperazin-4-yl)-propyl-1-phosphonic acid (“CPP”). Although these compounds have been reported (J. D. Kristensen, et al., Pain, 51:249-253 (1992); P. K. Eide, et al., Pain, 61:221-228 (1995); D. J. Knox, et al., Anaesh. Intensive Care 23:620-622 (1995); and M. B. Max, et al., Clin. Neuropharmacol 18:360-368 (1995)) to produce symptomatic relief in a number of neuropathies including postherpetic neuralgia, central pain from spinal cord injury, and phantom limb pain, widespread use of these compounds is precluded by their undesirable side effects. Such side effects at analgesic doses include psychotomimetic effects such as dizziness, headache, hallucinations, dysphoria, and disturbances of cognitive and motor function. Additionally, more severe hallucinations, sedation, and ataxia are produced at doses only marginally higher than analgesic doses.

NMDA receptors are heteromeric assemblies of subunits, of which two major subunit families: designated NR1 and NR2 have been cloned. Without being bound by theory, it is generally believed that the various functional NMDA receptors in the mammalian central nervous system (“CNS”) are only formed by combinations of NR1 and NR2 subunits, which respectively express glycine and glutamate recognition sites. The NR2 subunit family is in turn divided into four individual subunit types: NR2A, NR2B, NR2C, and NR2D. T. Ishii, et al., J. Biol. Chem., 268:2836-2843 (1993), and D. J. Laurie et al., Mol. Brain Res., 51:23-32 (1997) describe how the various resulting combinations produce a variety of NMDA receptors differing in physiological and pharmacological properties such as ion gating properties, magnesium sensitivity, pharmacological profile, as well as in anatomical distribution.

For example, while NR1 is found throughout the brain, NR2 subunits are differentially distributed. In particular, it is believed that the distribution map for NR2B lowers the probability of side effects while producing pain relief. For example, S. Boyce, et al., Neuropharmacology, 38:611-623(1999) describes the effect of selective NMDA NR2B antagonists on pain with reduced side effects.

5HT_{1B/1D} agonists (triptans) have shown to be efficacious in the acute treatment of migraine (Teall J, Tuchman M, Cutler N, Gross M, Willoughby E, Smith B, Jiang K, Reines S, Block G: Rizatriptan (MAXALT™) for the acute treatment of migraine and migraine recurrence. A placebo-controlled, outpatient study. Headache 1998;38:281-287). However, their action at 5HT_{1B} receptors produces therapeutic cerebral vasoconstriction with coronary vasoconstriction as an unwanted side effect. Consequently, all triptans are contraindicated in patients with known or suspected coronary artery disease. The present invention provides for the use of NR2B receptor antagonists having similar efficacy in the acute treatment of migraine without the cardiovascular liability of triptans.

Although triptans have shown efficacy in acute migraine, only about 40% of patients are free of headache pain by 2 hours (Teall, et al, supra). The present invention also provides for the concomitant use of NR2B receptor antagonists and triptans wherein the analgesic effects of the NR2B receptor antagonists complement the therapeutic effect of the triptan.

A traditional NSAID such as naproxen has been demonstrated to be effective in the prophylactic treatment of migraine attacks (Bellavance AJ, Meloche JP. A comparative study of naproxen sodium, pizotyline and placebo in migraine prophylaxis. Headache 1990;30(11):710-5; Welch KM, Ellis DJ, Keenan PA. Successful migraine prophylaxis with naproxen sodium. Neurology 1985 September;35(9):1304-10). The present invention provides for the use of NR2B receptor antagonists having similar efficacy to naproxen in migraine prophylaxis, but better tolerated than naproxen in chronic administration, which will improve compliance with therapy. In addition, as prophylactic agents usually provide 50% headache frequency reduction in less than half of treated patients (Stellar S, Ahrens SP, Meibohm AR, Reines SA. Migraine prevention with timolol. A double-blind crossover study. JAMA 1984;252:2576-80), the concomitant administration of an NR2B receptor antagonist with a COX-II inhibitor or montelukast provides a synergistic benefit in prophylaxis greater than that seen with prophylaxis monotherapy.

### SUMMARY OF THE INVENTION

The present invention encompasses a method for treating or preventing migraines in a mammalian patient in need of such treatment or prevention comprising administering to said patient an NR2B receptor antagonist in an amount that is effective to treat or prevent migraines. The invention also encompasses the combination of an NR2B antagonist with a cyclooxygenase-2 selective inhibitor, a calcitonin gene-related peptide receptor (CGRP) ligand, a leukotriene receptor antagonist or a 5_{HT 1B/1D} agonist for the treatment or prevention of migraines.

### DETAILED DESCRIPTION OF THE INVENTION

The present invention encompasses a method for treating or preventing migraines in a mammalian patient in need of such treatment or prevention comprising administering to said patient an NR2B receptor antagonist in an amount that is effective to treat or prevent migraines.

An embodiment of the invention encompasses the above method wherein the NR2B antagonist is administered at a dose ranging from about 0.1 mg to about 2500 mg.

Another embodiment of the invention encompasses the above method wherein the mammalian patient is human.

Another embodiment encompasses a method for treating migraines in a mammalian patient in need of such treatment comprising administering to said patient an NR2B receptor antagonist in an amount that is effective to treat migraines.

For purposes of this specification, treating migraines means relieving both the headache and the consequent associated symptoms of migraine. Treating migraines is synonymous with the acute treatment of migraines.

Another embodiment of the invention encompasses a method for preventing migraines in a mammalian patient in need of such prevention comprising administering to said patient an NR2B antagonist in an amount that is effective to prevent migraines For purposes of this specification, prevention of migraines means reducing the severity, the frequency or both the severity and frequency of migraine attacks. Preventing migraines is synonymous with migraine prophylaxis or the chronic treatment of migraines.

For purposes of this specification, migraine is meant to include migraine without aura, migraine with aura, migraine with typical aura, migraine with prolonged aura, familial hemiplegic migraine, basilar migraine, migraine aura without headache, migraine with acute onset aura, ophthalmoplegic migraine, retinal migraine, childhood periodic syndromes that may be precursors to or associated with migraine, benign paroxysmal vertigo of childhood, alternating hemiplegia of childhood, status migrainosus and migrainous infarction. Reference is made to the following: Headache Classification Committee of the International Headache Society: Classification ad diagnostic criteria for headache disorders, cranial neuralgias and facial pain. Cephalalgia. 1988;8(suppl 7): 1-96, which is hereby incorporated by reference in its entirety.

For purpose of this specification, an amount that is effective to treat or prevent migraines is that amount that will relieve the subject being treated of the symptoms of or reduce the severity and/or frequency of the migraine attack. The specific dose level and frequency of dosage may vary and will depend upon a variety of factors including the activity of the specific compounds used in combination, the metabolic stability and length of action of the compounds, the age, body weight, general health, sex diet, mode and time of administration, rate of excretion, the severity of the particular condition and the host undergoing therapy. However, dosage levels of the NR2B receptor antagonist on the order of about 0.001 mg/kg to about 30 mg/kg of body weight per day, are useful in the novel method of treatment. The compound may be administered on a regimen of up to 6 times per day, preferably 1 to 4 times per day. For the treatment of a migraine attack, the active ingredient may be administered orally, topically, parenterally, by inhalation, spray, rectally or intravaginally in formulations containing pharmaceutically acceptable carriers.

NR2B receptor antagonists are disclosed, for example, in the following published PCT patent publications: WO 01/32171, WO 01/32174, WO 01/32177, WO 01/32179, WO 01/32615 and WO 01/32634, all of which published on May 10, 2001 and all of which are hereby incorporated by reference in their entirety.

Compounds that are antagonists of the NR2B receptor also include compounds represented by Formula (I):

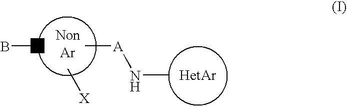

or pharmaceutically acceptable salts thereof, wherein

NonAr is a nonaromatic 5-7 membered ring containing 1 or 2 nitrogen ring atoms or an aza bicyclo octane ring;

HetAr is a 5 or 6 membered heteroaromatic ring containing 1-3 nitrogen ring atoms, or isoxazolyl, thiazolyl, thiadiazolyl, quinolinyl, quinazolinyl, purinyl, pteridinyl, benzimidazolyl, pyrrolopyrimidinyl, or imidazopyridinyl;

HetAr is optionally substituted with 1 or 2 substituents, each substituent independently is C₁₋₄alkyl, trifluoromethyl, hydroxy, hydroxyC₁₋₄alkyl, fluoro, chloro, bromo, iodo, cyano, methylsulfanyl, amino, nitro, (C₁₋₄alkyl)(C₁₋₂alkyl)NCH₂—, (C₁₋₂alkyl)HNCH₂—, or NH₂C(O);

A is —C₀₋₄alkyl-;

B is aryl(C₂)₀₋₃—O—C(O)—, heteroaryl(CH₂)₁₋₃—C(O)—, aryl(CH₂)₁₋₃—C(O)—, aryl-cyclopropyl-(O)—, heteroaryl(CH₂)₁₋₃—C(O)—, aryl(CH₂)₁₋₃-, heteroaryl(CH₂)₁₋₃-aryl(CH₂)₁₋₃—NH—C(O)—, aryl(CH₂)₁₋₃—NH—C(NCN), aryl(CH₂)₁₋₃—SO₂—, heteroaryl(CH₂)₁₋₃—SO₂—, wherein any of the aryl or heteroaryl is optionally substituted by 1-3 substituents, each substituent independently is C₁₋₄alkyl, C₃₋₆cycloalkyl, C₁₋₄alkoxy, trifluoromethyl, bromo, fluoro, or chloro; and

X is H, OH, F, C₁₋₄alkyl, C₁₋₄alkoxy, NH₂, or X taken with an adjacent bond is ═O.

The above compounds are disclosed in U.S. No. 60/271,100, filed on Feb. 23, 2001, which is hereby incorporated by reference in its entirety.

Compounds that are antagonists of the NR2B receptor also include compounds of Formula II:

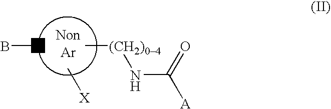

or a pharmaceutically acceptable salt thereof, wherein

NonAr is a nonaromatic 5-7 membered ring containing a) 1 nitrogen ring atom, b) 2 nitrogen ring atoms, c) 1 nitrogen and 1 oxygen ring atom, or d) 1 nitrogen and 1 sulfur ring atom, wherein the remaining ring atoms are carbon;

A is a phenyl optionally substituted with 1-3 substituents, each substituent independently is C₁₋₄alkyl, C₃₋₇cycloalkyl, —CF₃, halogen, —OH, —CN, —NH₂, —O—C₁₋₄alkyl, —NH—C₁₋₄alkyl, or —NHSO₂—C₁₋₄alkyl; or

A is pyrrolyl, imidazolyl, pyrazolyl, triazolyl, thiophenyl, thiazolyl, thiadiazolyl, oxazolyl, or isoxazolyl, each optionally substituted with 1-3 substituents, each substituent independently is —C₁₋₄alkyl, —C₃₋₇cycloalkyl, —CF₃, halogen, —OH, —CN, phenyl, —C₁₋₄hydroxyalkyl; or

A is pyridyl, pyradazinyl, pyrimidinyl, or pyrazinyl, each optionally substituted with 1-3 substituents, each substituent independently is —C₁₋₄alkyl, —C₃₋₇cycloalkyl, —CF₃, halogen, —OH, —CN, phenyl, —C₁₋₄hydroxyalkyl, —C₁₋₄alkoxy, (CH₃)₂N—(CH₂)₂—NH—, —C₀₋₄alkyl-N(C₀₋₄alkyl)(C₀₋₄alkyl), dimethoxyphenyl-CH₂—NH—, or the substituent taken with a neighboring bond is ═O; or

A is pyrrolophenyl, imidazolophenyl, pyrazolophenyl, triazolophenyl, pyridinoimidazolyl, naphthyridinyl, tetrahydrocyclopentopyrazolyl, quinolinyl, pyrimidinopyrazololyl, benzothiazolyl, benzoimidazolyl, or purinyl, each optionally substituted with 1-3 substituents, each substituent independently is —C₁₋₄alkyl, —C₃₋₇cycloalkyl, —CF₃, halogen, —OH, or —CN;

B is aryl(CH₂)₀₋₃—O—C(O)—, heteroaryl(CH₂)₁₋₃—O—C(O), indanyl(CH₂)₀₋₃—O—C(O)—, aryl(CH₂)₁₋₃—O—C(O)—, aryl-cyclopropyl-C(O)—, heteroaryl(CH₂)₁₋₃—C(O), aryl(CH₂)₁₋₃—, heteroaryl(CH₂)₁₋₃—, aryl(CH₂)₁₋₃—NH—C(O)—, aryl(CH₂)₁₋₃—NH—C(NCN)—, aryl(CH₂)₁₋₃—SO₂—, or heteroaryl(CH₂)₁₋₃—SO₂— wherein any of the aryl or heteroaryl is optionally substituted by 1-3 substituents, each substituent independently is C₁₋₄alkyl, C₃₋₆cycloalkyl, C₁₋₄alkoxy, trifluoromethyl, bromo, fluoro, or chloro; or

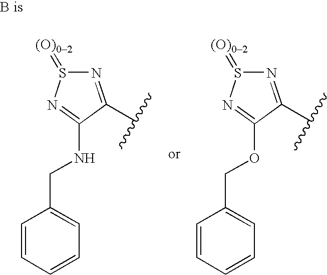

wherein the phenyl is optionally substituted by 1-3 substituents, each substituent independently is C₁₋₄alkyl, C₃₋₆cycloalkyl, C₁₋₄alkoxy, trifluoromethyl, bromo, fluoro, or chloro; and

X is H, OH, F, C₁₋₄alkyl, or C₁₋₄alkoxy.

The above compounds are disclosed in U.S. No. 60/281,166, filed on Apr. 3, 2001, which is hereby incorporated by reference in its entirety.

Compounds that are NR2B receptor antagonists also include compounds of Formula III:

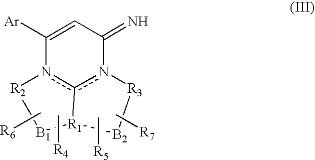

or a pharmaceutically acceptable salt thereof, wherein

i) Ar is an aromatic group, the aromatic group being phenyl, naphthyl, pyridinyl, pyridazinyl, pyrimidinyl, pyrazinyl, imidazolyl, quinoxalinyl, furyl, thienyl, pyrrolyl, benzimidazolyl, indolyl, quinolinyl, isoquinolinyl, pyrazolyl, indazolyl, oxazolyl, isoxazolyl, thiazolyl, isothiazolyl, oxadiazolyl, thiadiazolyl, triazolyl, tetrazolyl, imidazolyl, benzthienyl, or benzofuryl, the aromatic group optionally substituted by one or two substituents, each substituent independently is halogen, C₁₋₄alkyl, or oxyC₁₋₄alkyl;

ii) R₁ is a phenyl; or —CH₂—, —NH—, —NR₄—, —NR₅—, or ═N-when optionally connected either via B₁ to R₂ or via B₂ to R₃;

iii) R₂ is a phenyl group, a C₁₋₄alkylphenyl group, or absent, wherein the groups optionally may be substituted by one or two substituents, each substituent is independently halogen, C₁₋₄alkyl, or oxy₁₋₄alkyl; R₂ optionally is —CH₂— or ═CH— connected via B₁ to R₁;

iv) R₃ is a phenyl group, a C₁₋₄alkylphenyl group, or absent, wherein the groups optionally may be substituted by one or two substituents, each substituent is independently halogen, C₁₋₄alkyl, or oxyC₁₋₄alkyl; R₃ optionally is —CH₂— or ═CH— connected via B₂ to R₁;

v) R₄ is a phenyl group, a C₁₋₄alkylphenyl group, or absent, wherein the groups optionally may be substituted by one or two substituents, each substituent is independently halogen, C₁₋₄alkyl, or oxyC₁₋₄alkyl;

vi) R₅ is a phenyl group, a C₁₋₄alkylphenyl group, or absent, wherein the groups optionally may be substituted by one or two substituents, each substituent is independently halogen, C₁₋₄alkyl, or oxyC₁₋₄alkyl;

vii) R₆ is a phenyl group, a C₁₋₄alkylphenyl group, or absent;

viii) R₇ is a phenyl group, a C₁₋₄alkylphenyl group, or absent;

ix) B₁ is —CH₂—, ═CH—, —CH₂CH₂—, —CH═CH—, or absent; and

x) B₂ is —CH₂—, ═CH—, —CH₂CH₂—, —CH═CH—, or absent.

Examples of the above compounds are as follows:

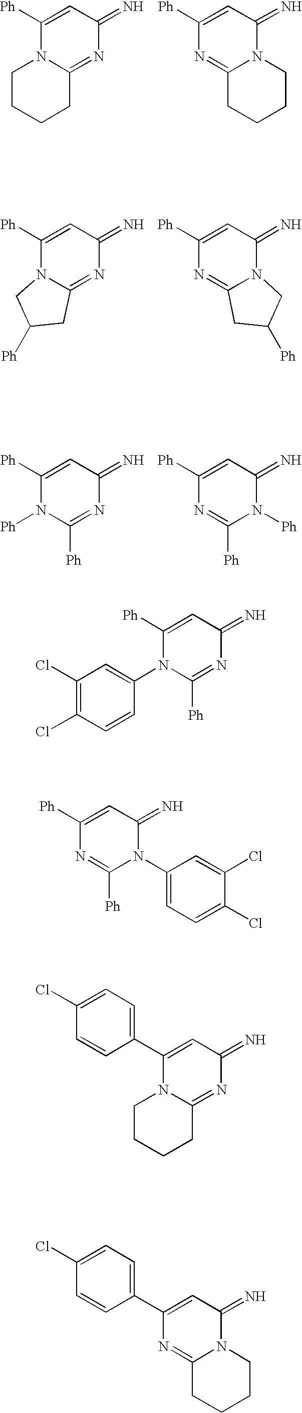

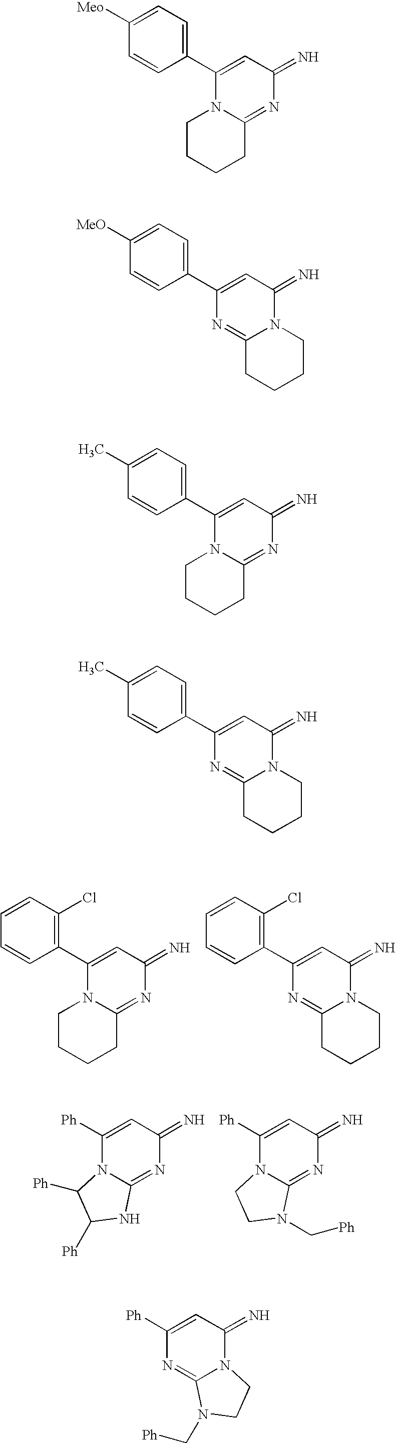

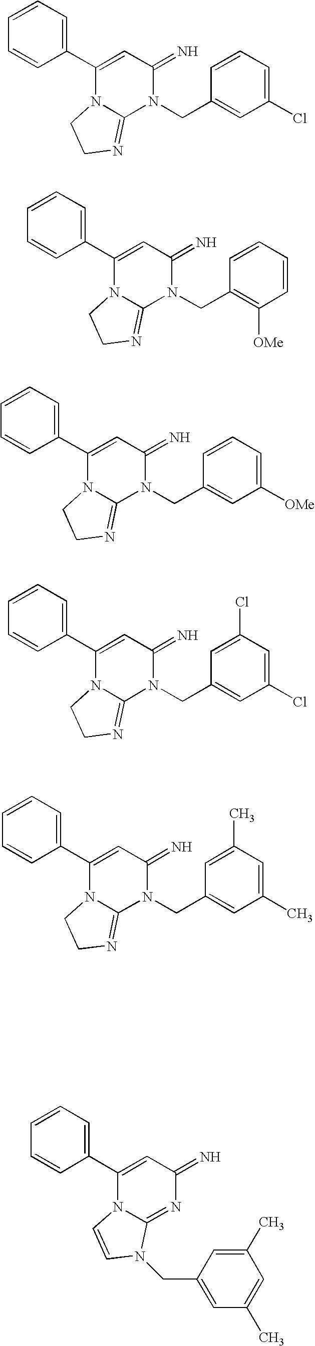

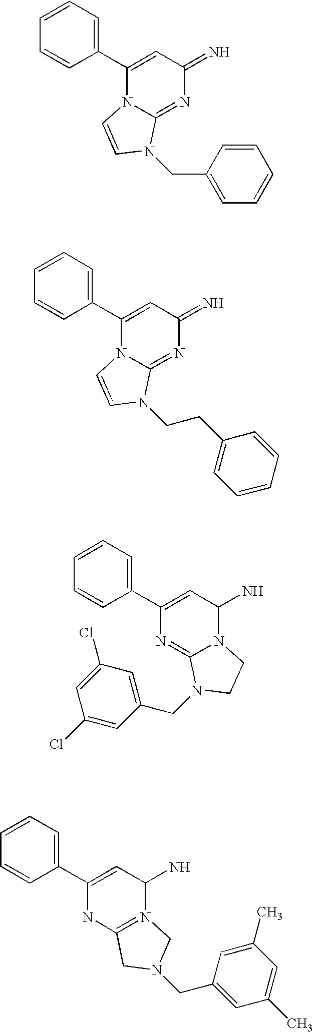

or pharmaceutically acceptable salts thereof,

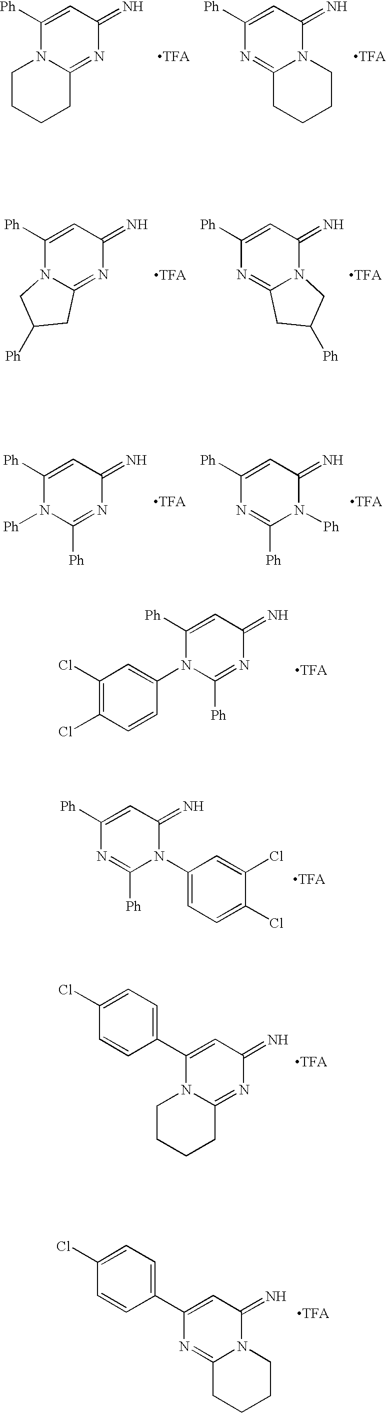

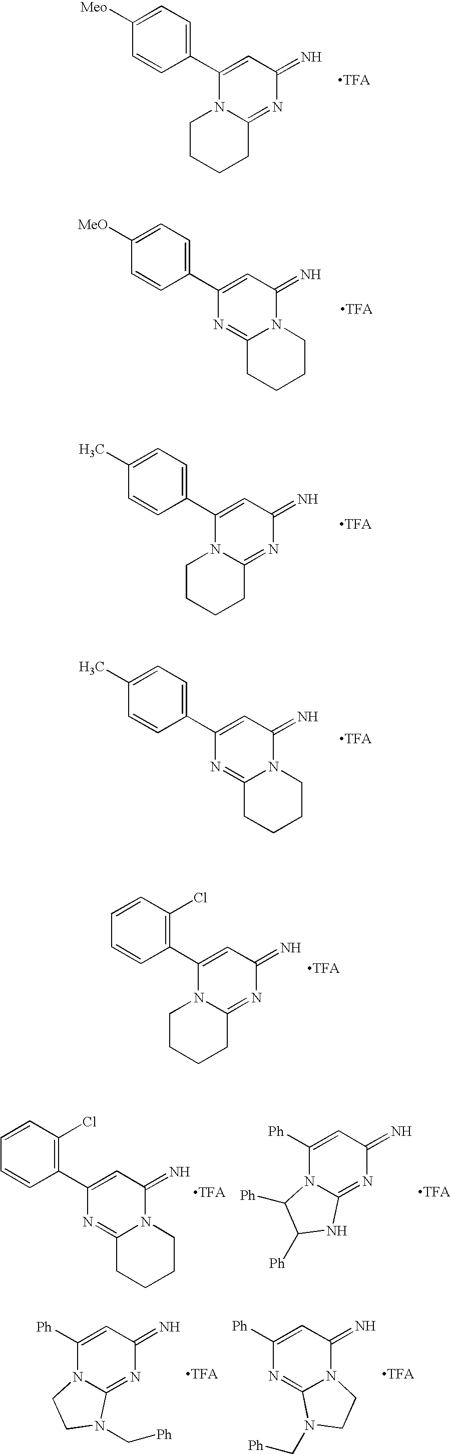

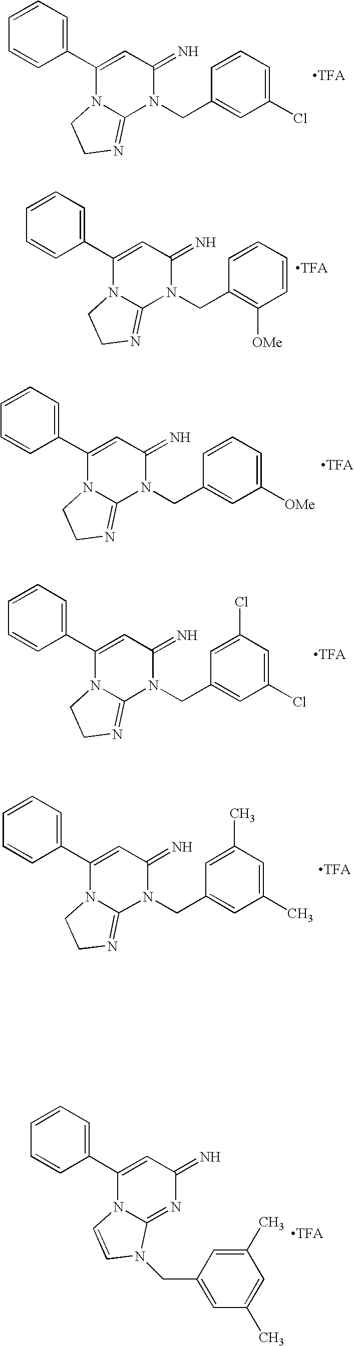

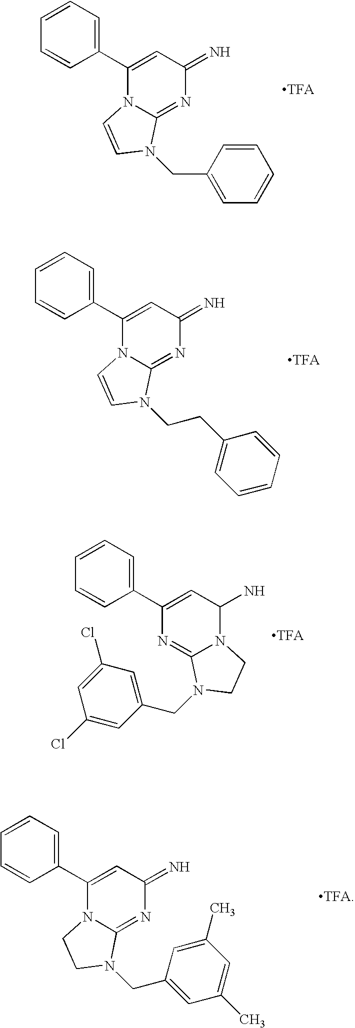

The above compounds are disclosed in U.S. No. 60/214,654, filed on Jun. 26, 2000 and WO 02/00629, published on Jan. 3, 2002, which are hereby incorporated by reference in its entirety.

The NR2B antagonists described herein may contain one or more asymmetric centers and may thus give rise to diastereomers and optical isomers. The present invention includes all such possible diastereomers as well as their racemic mixtures, their substantially pure resolved enantiomers, all possible geometric isomers, and pharmaceutically acceptable salts thereof. Mixtures of stereoisomers as well as isolated specific stereoisomers are also included. During the course of the synthetic procedures used to prepare such compounds, or in using racemization or epimerization procedures known to those skilled in the art, the products of such procedures can be a mixture of stereoisomers.

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When the compound of the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particularly preferred are citric, hydrobromic, hydrochloric, maleic, phosphoric, sulfuric, and tartaric acids.

The pharmaceutical compositions of the present invention comprise an NR2B receptor antagonist (or pharmaceutically acceptable salts thereof) as an active ingredient, a pharmaceutically acceptable carrier and optionally other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the NR2B receptor antagonist, or pharmaceutically acceptable salts thereof, of this invention can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the NR2B receptor antagonist, or pharmaceutically acceptable salts thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and a compound or a pharmaceutically acceptable salt of the NR2B receptor antagonist. The NR2B receptor antagonist, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 1 mg to about 500 mg of the active ingredient and each cachet or capsule preferably containing from about 1 mg to about 500 mg of the active ingredient.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared, utilizing an NR2B receptor antagonist of this invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in moulds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing an NR2B receptor antagonist, or pharmaceutically acceptable salts thereof, may also be prepared in powder or liquid concentrate form.

NR2B antagonists may also be administered in combination with other agents for the treatment or prevention of migraines. Such administration may either be in unit dosage form or concomitantly. All conventional anti-migraine agents are used in conjunction with the NR2B antagonist at conventional doses that are determined by the skilled clinician. These compounds are known and normal daily dosages are well established. Typically, the individual daily dosages for these combinations may range from about one-fifth of the minimally recommended clinical dosages to the maximum recommended levels for the entities when they are given alone. Precise dosages are left to the discretion of the physician

Thus, in further aspects, the invention encompasses a method for treating or preventing migraines in a mammalian patient in need of such treatment or prevention comprising concomitantly administering a calcitonin gene-related peptide receptor (CGRP) ligand with a NR2B receptor antagonist in amounts that are effective to treat or prevent migraines. CGRP ligands are disclosed, for example, in the following published patent applications: WO 00/18764 published on Apr. 6, 2000, WO 01/10425 published on Feb. 15, 2001, WO 00/55154 published on Sep. 21, 2000, and WO 98/11128 published on Mar. 19, 1998, all of which are hereby incorporated by reference in their entirety.

When administered in combination, either a single or as a separate pharmaceutical composition for the treatment or prevention of migraine, the NR2B receptor antagonist and the CGRP ligand are presented in a ratio that is consistent with the manifestation of the desired effect. In particular, the ratio by weight of the NR2B receptor antagonist to the CGRP ligand will suitably be approximately 1 to 1. Preferably, this ratio will be between 0.001 to 1 and 1000 to 1, and especially between 0.01 to 1 and 100 to 1.

For purposes of the present invention, intravenous dosages or oral dosages of CGRP ligands will range between about 0.001 to 5 mg/kg and 0.01 to 50 mg/kg, respectively. The compound may be administered on a regimen of up to 6 times per day, preferably 1 to 4 times per day.

The invention also encompasses a method for treating or preventing migraines in a mammalian patient in need of such treatment or prevention comprising concomitantly administering a cyclooxygenase-2 selective inhibiting compound with a NR2B receptor antagonist in amounts that are effective to treat or prevent migraines. Examples of cyclooxygenase-2 selective inhibiting compounds useful in the methods described herein include Celebrex® (celecoxib), VIOXX® (rofecoxib), etoricoxib (WO98/03484), valdecoxib (U.S. Pat. No. 5,663,272), parecoxib (U.S. Pat. No. 5,932,598), COX189, BMS347070, ABT963, CS502, GW406381, JTE522, which has the following structure:

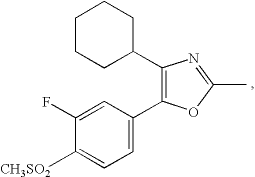

as well as the compounds disclosed in U.S. Pat. No. 6,020,343, including the following:

(1) 2-(3,4-difluorophenoxy)-3-(4-methylsulfonylphenyl)-cyclopent-2-enone,

(2) 3-(5-Benzothiophenyloxy)-5,5-dimethyl-4-(4-(methylsulfonyl) phenyl)-5H-furan-2-one,

(3) 5,5-dimethyl-4-(4-methylsulfonyl-phenyl)-3-(pyridyl-4-oxy)-5H-furan-2-one,

(4) 5,5-dimethyl-4-(4-methylsulfonyl-phenyl)-3-(pyridyl-3-oxy)-5H-furan-2-one,

(5) 3-(2-Methyl-5-pyridyloxy)-5,5-dimethyl-4-(4-(methylsulfonyl) phenyl)-5H-furan-2-one,

(6) 3(2-Fluoro-4-trifluoromethyl)phenoxy-4-(4-methylsulfonyl)phenyl)-5,5-dimethyl-5H-furan-2-one,

(7) 3-(5-Chloro-2-pyridylthio)-5,5-dimethyl-4-(4-methylsulfonyl)phenyl-5H-furan-2-one,

(8) 2-(3,5-Difluorophenoxy)-3-(4-methylsulfonylphenyl)-cyclopent-2-enone,

(9) 3-(2-Pyrimidinoxy)-5,5-dimethyl-4-(4-methylsulfonyl)phenyl-5H-furan-2-one,

(10) 3-(3-Methyl-2-pyridyloxy)-5,5-dimethyl-4-(4-methylsulfonyl)phenyl-5H-furan-2-one,

(11) 3-(3-Chloro-5-pyridyloxy)-5,5-dimethyl-4-(4-(methylsulfonyl) phenyl)-5H-furan-2-one,

(12) 3-(3-(1,2,5-Thiadiazolyl)oxy)4-(4-(methylsulfonyl)phenyl)-5,5-dimethyl-5H-furan-2-one,

(13) 3-(5-Isoquinolinoxy)-5,5-dimethyl-4-(4-methylsulfonyl)phenyl-5H-furan-2-one,

(14) 3-(6-Amino-2-pyridyloxy)-5,5-dimethyl-4-(4-(methylsulfonyl) phenyl)-5H-furan-2-one,

(15) 3-(3-Chloro-4-fluoro)phenoxy-4-(methylsulfonyl)phenyl)-5,5-dimethyl-5H-furan-2-one,

(16) 3-(6-Quinolinoxy)-5,5-dimethyl-4-(4-(methylsulfonyl)phenyl)-5H-furan-2-one,

(17) 3-(5-Nitro-2-pyridyloxy)-5,5-dimethyl-4-(4-methylsulfonyl)phenyl-5H-furan-2-one,

(18) 3-(2-Thiazolylthio)-5,5-dimethyl-1(4-(methylsulfonyl)phenyl)-5H-furan-2-one,

(19) 3-(3-Chloro-5-pyridyloxy)-5,5-dimethyl-4-(4-(methylsulfonyl) phenyl)-5H-furan-2-one,

(20) 5,5-Dimethyl-4(4-methylsulfonylphenyl)-3-(2-propoxy)-5H-furan-2-one,

(21) 3-(3-Trifluoromethyl)phenoxy-4-(4-methylsulfonyl)phenyl)-5,5-dimethyl-5H-furan-2-one,

(22) 5,5-Dimethyl-(4-(4-methylsulfonyl)phenyl)-3-(piperidine-1-carbonyl)-5-H-furan-2-one,

(23) 5,5-Dimethyl-3-(2-Butoxy)-4-(4-methylsulfonylphenyl)-5H-furan-2-one,

(24) 5,5-Dimethyl-4-(4-methylsulfonylphenyl)-3-(3-pentoxy)-5H-furan-2-one,

(25) 2-(5-Chloro-2-pyridyloxy)-3-(4-methylsulfonyl)phenylcyclopent-2-enone,

(26) 3-(4-Methyl-2-pyridyloxy)-5,5-dimethyl-4-(4-methylsulfonyl)phenyl-5H-furan-2-one,

(27) (5R)-3-(3,4-Difluorophenoxy)-5-ethyl-5-methyl-4-(4-methylsulfonyl)phenyl-5H-furan-2-one,

(28) (5R)-3-(4-Chlorophenoxy)-5-ethyl-5-methyl-4-(4-methylsulfonyl)phenyl-5H-furan-2-one,

(29) 3-(2-Methyl-3-pyridyloxy)-5,5-diethyl-4-(4-methylsulfonyl)phenyl-5H-furan-2-one,

(30) 3-(4-Methyl-5-nitro-2-pyridyloxy)-5,5-dimethyl-4-(4-methylsulfonyl)phenyl-5H-furan-2-one,

(31) 3-(5-Chloro-4-methyl-2-pyridyloxy)-5,5-dimethyl-4-(4-methylsulfonyl)phenyl-5H-furan-2-one,

(32) 3-(5-Fluoro-4-methyl-2-pyridyloxy)-5,5-dimethyl-4-(4-methylsulfonyl)phenyl-5H-furan-2-one,

(33) 3-(3-Chloro-2-pyridyloxy)-5,5-dimethyl-4-(4-methylsulfonyl)phenyl-5H-furan-2-one,

(34) 3-(4-Fluorophenoxy)-5-methyl(4-methylsulfonyl)phenyl-5-propyl-5H-furan-2-one,

(35) 3-(N,N-Diethylamino)-5,5-dimethyl-4-(4-(methylsulfonyl) phenyl)-5H-furan-2-one,

(36) 5,5-dimethyl-4-(4-methylsulfonyl-phenyl)-3-(3,5-dichloro-2-pyridyloxy)-5H-furan-2-one,

(37) (5R)-3-(4-Bromophenoxy)-5-ethyl-5-methyl-4-(4-methylsulfonyl)phenyl-5H-furan-2-one,

(38) (5R)-3-(4-Methoxyphenoxy)-5-ethyl-5-methyl-4-(4-methylsulfonyl)phenyl-5H-furan-2-one,

(39) (5R)-3-(5-Chloro-2-pyridyloxy)-5-methyl-4-(4-methylsulfonyl)phenyl-5-(2,2,2-trifluoroethyl)-5H-furan-2-one,

(40) 3-(5-Chloro-2-pyridyloxy)-5-methyl-4-(4-methylsulfonyl)phenyl-5-propyl-5H-furan-2-one,

(41) 3-(1-Cyclopropyl-ethoxy)-5,5-dimethyl-4-(4-methyl sulfonyl)phenyl)-5H-furan-2-one,

(42) 5-Methyl-4-(4-(methylsulfonyl)phenyl)-3-(2-(propoxy)-5-(2-trifluoroethyl)-5H-furan-2-one,

(43) 5(R)-5-ethyl-5-methyl-4-(4-(methylsulfonyl)phenyl)-3-(2-propoxy)-5H-furan-2-one,

(44) 5,5-dimethyl-3-(2,2-dimethylpropyloxy)-4-(4-(methylsulfonyl)phenyl)-5H-furan-2-one,

(45) 5(R) 3-(1-cyclopropyl-ethoxy)-5-ethyl-5-methyl-4-(4-(methyl sulfonyl)phenyl-5H-furan-2-one,

(46) 5(S) 5-Ethyl-5-methyl-4-(4-(methylsulfonyl)phenyl-3-(2-propoxy)-5H-furan-2-one,

(47) 3-(1-cyclopropylethoxy)-5,5-dimethyl-4-(4-(methylsulfonyl)phenyl)-5H-furan-2-one,

(48) 3-(1-cyclopropylethoxy)-5,5-dimethyl-4-(4-(methylsulfonyl)phenyl)-5H-furan-2-one,

(49) 3-(cyclopropylmethoxy)-5,5-dimethyl-4-(4-(methylsulfonyl) phenyl-5H-furan-2-one,

(50) 5,5-dimethyl-3-(isobutoxy)-4-(4-(methylsulfonyl)phenyl)-5H-furan-2-one,

(51) 3-(4-Bromophenoxy)-5,5-dimethyl-4-(4-(methylsulfonyl)phenyl)-5H-furan-2-one,

(52) 3-(2-Quinolinoxy)-5,5-dimethyl-4-(4-(methylsulfonyl)phenyl)-5H-furan-2-one,

(53) 3-(2-Chloro-5-pyridyloxy)-5,5-dimethyl-4-(4-(methylsulfonyl)phenyl)-5H-furan-2-one,

(54) 3-(6-benzothiazolyloxy)-5,5-dimethyl-(4-(methylsulfonyl) phenyl)-5H-furan-2-one,

(55) 3-(6-Chloro-2-pyridyloxy)-5,5-dimethyl-4-(4-(methylsulfonyl) phenyl)-5H-furan-2-one,

(56) 3-(4-Quinazolyloxy)-5,5-dimethyl-4-(4-(methylsulfonyl)phenyl)-5H-furan-2-one,

(57) (5R)-3-(5-Fluoro-2-pyridyloxy)-5-ethyl-5-methyl-4-(4-methylsulfonyl)phenyl-5H-furan-2-one,

(58) (5R)-3-(4-Fluorophenoxy)-5-ethyl-5-methyl-4-(4-methylsulfonyl)phenyl-5H-furan-2-one,

(59) (5R)-3-(5-Fluoro-2-pyridyloxy)-5-methyl-4-(4-methylsulfonyl)phenyl-5-(2,2,2-trifluoroethyl)-5H-furan-2-one,

(60) 3-(1-Isoquinolinyloxy)-5,5-dimethyl-4-(methylsulfonyl)phenyl-5H-furan-2-one,

(61) (5R)-3-(4-fluorophenoxy)-5-methyl-4-(4-methylsulfonyl)phenyl-5-(2,2,2-trifluoroethyl)-5H-furan-2-one,

(62) 3-(3-Fluoro-2-pyridyloxy)-5,5-dimethyl-4-(4-methylsulfonyl) phenyl-5H-furan-2-one,

(63) (5R)-3-(3,4-difluorophenoxy)-5-methyl-4-(4-methylsulfonyl) phenyl-5-(2,2,2-trifluoroethyl)-5H-furan-2-one,

(64) (5R)-3-(5-chloro-2-pyridyloxy)-5-ethyl-5-methyl-4-(4-methylsulfonyl)phenyl-5H-furan-2-one,

(65) 3-(3,4-difluorophenoxy)-5-methyl-5-trifluoromethyl-4-(4-methylsulfonyl)phenyl-5H-furan-2-one,

(66) 3-(3,4-Difluorophenoxy)-5-methyl-4-(4-(methylsulfonyl)phenyl)-5-propyl-5H-furan-2-one,

(67) 3-Cyclobutyloxy-5,5-dimethyl-4-(4-methylsulfonylphenyl-5H-furan-2-one,

(68) 3-(1-Indanyloxy)-5,5-dimethyl-4-(4-(methylsulfonyl)phenyl)-5H-furan-2-one,

(69) 3-(2-Indanyloxy)-5,5-dimethyl-4-(4-methylsulfonyl)phenyl)-5H-furan-2-one,

(70) 3-Cyclopentyloxy-5,5-dimethyl-4-(4-methylsulfonylphenyl)5H-furan-2-one,

(71) 3-(3,3-Dimethylcyclopentyloxy)-5,5-dimethyl-4-(4-methylsulfonyl-phenyl)-5H-furan-2-one,

(72) 3-Isopropoxy-5-methyl-4-(4-methylsulfonylphenyl)-5-propyl-5H-furan-2-one,

(73) 3-(2-Methoxy-5-pyridyloxy)-5,5-dimethyl-4-(4-methylsulfonyl)phenyl-5H-furan-2-one,

(74) 3-(5-Methyl-2-pyridyloxy)-5,5-dimethyl-4-(4-methylsulfonyl)phenyl-5H-furan-2-one,

(75) (5RS)-3-(3,4-Difluorophenoxy)-5-methyl-4-(4-methylsulfonyl)phenyl-5-(2,2,2-trifluoroethyl)-5H-furan-2-one,

(76) 3-(3-Chloro-4-methoxyphenoxy)-5,5-dimethyl-4-(4-methylsulfonyl)phenyl-5H-furan-2-one,

(77) (5R)-3-(3-Chloro-4-methoxyphenoxy)-5-ethyl-5-methyl-4-(4-methylsulfonyl)phenyl-5H-furan-2-one,

(78) (5R)-3-(4-Chlorophenoxy)-5-trifuoroethyl-5-methyl-4-(4-methylsulfonyl)phenyl-5H-furan-2-one,

(79) (5R)-3-(4-Bromophenoxy)-5-trifuoroethyl-5-methyl-4-(4-methylsulfonyl)phenyl-5H-furan-2-one,

(80) 5-Cyclopropylmethyl-3-(3,4-difluorophenoxy)-5-methyl-(4-methylsulfonyl)phenyl-5H-furan-2-one,

(81) (5R)-3-(3-Fluorophenoxy)-5-ethyl-5-methyl-4-(4-methylsulfonyl)phenyl-5H-furan-2-one,

(82) (5R)-3-(4-Chloro-3-fluorophenoxy)-5-ethyl-5-methyl-4-(4-methylsulfonyl)phenyl-5H-furan-2-one,

(83) (5R)-3-Phenoxy-5-ethyl-5-methyl-4-(4-methylsulfonyl)phenyl-5H-furan-2-one,

(84) (5R)-3-(4-Chloro-3-methylphenoxy)-5-ethyl-5-methyl-4-(4-methylsulfonyl)phenyl-5H-furan-2-one,

(85) 3-(4-Chloro-3-methylphenoxy)-5-5-dimethyl-4-(4-methylsulfonyl)phenyl-5H-furan-2-one,

(86) (5R)-3-(5-bromo-2-pyridyloxy)4-(4-methylsulfonylphenyl)-5-methyl-5-(2,2,2-trifluoroethyl)-5H-furan-2-one,

(87) (5R)-3-(5-bromo-2-pyridyloxy)-4-(4-methylsulfonylphenyl)-5-ethyl-5-methyl-5H-furan-2-one,

(88) 3-(5-chloro-6-methyl-2-pyridyloxy)-5,5-dimethyl-4-(4-methylsulfonyl)phenyl-5H-furan-2-one,

(89) 3-(5-cyclopropyl-2-pyridyloxy)-5,5-dimethyl-4-(4-methylsulfonyl)phenyl-5H-furan-2-one,

(90) 3-(1-cyclopropylethoxy)-4-(4-methylsulfonyl)phenyl-5H-furan-2-one, and

(91) 3-(cyclopropylmethoxy)-4-(4-methylsulfonyl)phenyl-5H-furan-2-one.

All of the aforesaid patents and published applications are hereby incorporated by reference in their entirety. A preferred cyclooxygenase-2 selective inhibiting compound for the present invention is refecoxib. Another preferred cyclooxygenase-2 selective inhibiting compound for the present invention is etoricoxib.

When administered in combination, either a single or as a separate pharmaceutical composition for the treatment or prevention of migraine, the NR2B receptor antagonist and the cyclooxygenase-2 selective inhibiting compound are presented in a ratio that is consistent with the manifestation of the desired effect. In particular, the ratio by weight of the NR2B receptor antagonist to the COX-2 inhibitor will suitably be approximately 10 to 1. Preferably, this ratio will be between 0.001 to 1 and 1000 to 1, and especially between 0.01 to 1 and 100 to 1.

For purposes of the present invention, the COX-2 inhibitor may be administered at a dosage level up to conventional dosage levels for such analgesics, but preferably at a reduced level in accordance with the present invention. Suitable dosage levels will depend upon the analgesic effect of the chosen COX-2 inhibitor, but typically suitable levels will be about 0.001 to 25 mg/kg per day, preferably 0.005 to 10 mg/kg per day, and especially 0.005 to 5 mg/kg per day. The compound may be administered on a regimen of up to 6 times per day, preferably 1 to 4 times per day.

The invention also encompasses a method for treating or preventing migraines in a mammalian patient in need of such treatment or prevention comprising concomitantly administering a 5HT_{1B/1D} agonist with a NR2B receptor antagonist in amounts that are effective to treat or prevent migraines. Examples of 5HT_{1B/1D} agonists are rizatriptan (EP 0,497,512), sumatriptan (GB 2,162,522), naratriptan (GB 2,208,646), zolmitriptan (WO91/18897), eletriptan (WO92/06973), and almotriptan (WO94/02460). All of the aforesaid patents and published applications are hereby incorporated by reference in their entirety.

The preferred 5HT_{1B/1D} agonist for use in this invention is rizatriptan, which is N,N-dimethyl-2-[5-(1,2,4-triazol-1-ylmethyl)-1H-indol-3-yl]ethylamine, the benzoate salt thereof being particularly preferred.

When administered in combination, either a single or as a separate pharmaceutical composition for the treatment or prevention of migraine, the NR2B receptor antagonist and the 5HT_{1B/1D} agonist are presented in a ratio that is consistent with the manifestation of the desired effect. In particular, the ratio by weight of the NR2B receptor antagonist to the 5HT_{1B/1D} agonist will suitably be approximately 10 to 1. Preferably, this ratio will be between 0.001 to 1 and 1000 to 1, and especially between 0.01 to 1 and 100 to 1.

A suitable dosage of the 5HT_{1B/1D} agonist for purposes of the present invention is about 0.01 to 250 mg/kg per day, preferably about 0.05 to 100 mg/kg per day, and especially about 0.05 to 5 mg/kg per day. The 5HT_{1B/1D} agonist may be administered on a regimen of 1 to 4 times per day.

The invention also encompasses a method for treating or preventing migraines in a mammalian patient in need of such treatment or prevention comprising concomitantly administering a leukotriene receptor antagonist with a NR2B receptor antagonist in amounts that are effective to treat or prevent migraines. Various leukotriene receptor antagonist drugs are known in the art. The two most widely used leukotriene receptor antagonists are (i) zafirlukast, which is sold under the tradename ACCOLATE®), and (ii) montelukast, sold under the tradename SINGULAIR®. Other leukotriene receptor antagonist drugs have also been reported in the literature, which fall generally into two categories: (1) leukotriene receptor-blocking drugs, such as pranlukest, BAY×7195, LY293111, ICI 204,219, and ONO-1078; and, (2) drugs which inhibit the biosynthesis of leukotrienes, such as BAY×1005, MK-886, MK0591, ZD2138, and zileuton. A preferred leukotriene receptor antagonist is montelukast.

When administered in combination, either a single or as a separate pharmaceutical composition, for the treatment or prevention of migraine, the NR2B receptor antagonist and the leukotriene receptor antagonist are presented in a ratio that is consistent with the manifestation of the desired effect. In particular, the ratio by weight of the NR2B receptor antagonist to the leukotriene receptor antagonist will suitably be approximately 10 to 1. Preferably, this ratio will be between 0.001 to 1 and 1000 to 1, and especially between 0.01 to 1 and 100 to 1.

For purpose of the present invention, leukotriene receptor antagonists may be administered at a dosage of about 0.001 mg to about 100 mg per kg body weight of a mammal, preferably 0.01 mg to about 10 mg per kg, and most preferably 0.1 to 1 mg per kg, in single or divided doses.

The invention also encompasses a pharmaceutical composition comprising an NR2B receptor antagonist and a CGRP receptor ligand in combination with a pharmaceutically acceptable carrier.

The invention also encompasses a pharmaceutical composition comprising an NR2B receptor antagonist and a 5HT_{1B/1D} agonist in combination with a pharmaceutically acceptable carrier.

The invention also encompasses a pharmaceutical composition comprising an NR2B receptor antagonist and a leukotriene receptor antagonist in combination with a pharmaceutically acceptable carrier.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, effective dosages other than the particular dosages as set forth herein above may be applicable as a consequence of variations in the responsiveness of the mammal being treated for any of the indications with the compounds of the invention indicated above. Likewise, the specific pharmacological responses observed may vary according to and depending upon the particular active compounds selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims that follow and that such claims be interpreted as broadly as is reasonable.

### EXAMPLES

The following exemplify NR2B receptor antagonists as well as methods for synthesis:

Intermediates:

Intermediate A1a:

Carbonic acid 2,5-dioxo-pyrrolidin-1-yl ester 4-methyl-benzyl ester

Disuccinimidyl carbonate (5.03 g, 19.65 mmol) in 30 mL MeCN and 30 mL DCM was treated with 4-methylbenzyl alcohol (2.4 g, 19.6 mmol) followed by DMAP (1.20 g, 9.82 mmol). The resulting cloudy reaction mixture cleared over 2 min, stirred overnight at rt, then poured into 100 mL water and partitioned. The organic layer was dried over anhydrous sodium sulfate and the solvent evaporated. The solid thus obtained was stirred with approx. 25 mL ether, filtered, washed with a small volume of ether and dried to yield carbonic acid 2,5-dioxo-pyrrolidin-1-yl ester 4-methyl-benzyl ester as a white solid.

Ref: Chem. Pharm. Bull., 38(1):110-115(1990).

The following compounds were prepared in the manner similar to that described above for INTERMEDIATE A1a:

Intermediate A1b:

Carbonic acid 2,5-dioxo-pyrrolidin-1-yl ester 4-chloro-benzyl ester

Intermediate A1c:

Carbonic acid 2,5-dioxo-pyrrolidin-1-yl ester 4-fluoro-benzyl ester

Intermediate A1d:

Carbonic acid 2,5-dioxo-pyrrolidin-1-yl ester 4-ethyl-benzyl ester

Intermediate A1e:

Carbonic acid 2,5-dioxo-pyrrolidin-1-yl ester 4-isopropyl-benzyl ester

Utilizing the carbonic acid derivatives described above for INTERMEDIATES A1a-A1e, and following the procedure described below in EXAMPLE A13, step 1, the following INTERMEDIATES A2a-A2e were obtained

Intermediate A2a:

4-Methylbenzyl 4-(aminomethyl)piperidine-1-Carboxylate

Intermediate A2b:

4-Chlorobenzyl 4-(aminomethyl)piperidine-1-carboxylate

Intermediate A2c:

4-Fluorobenzyl 4-(aminomethyl)piperidine-1-carboxylate

Intermediate A2d:

4-Ethylbenzyl 4-(aminomethyl)piperidine-1-carboxylate

Intermediate A2e:

4-Isopropylbenzyl 4-(aminomethyl)piperidine-1-carboxylate

### Example A1

Benzyl 4-[(4-pyridinylamino)methyl]-1-piperidinecarboxylate:

Step 1:

Benzyl 4-[(4-pyridinylamino)carbonyl]-1-piperidinecarboxylate

In DMF (5 mL), 1-[(benzyloxy)carbonyl]-4-piperidinecarboxylic acid (P. E. Maligres et al., Tetrahedron, 53:10983(1997)) (1.00 g, 3.80 mmol), 4-aminopyridine (572 mg, 6.08 mmol), EDC (801 mg, 4.18 mmol), and HOAt (569 mg, 4.18 mmol) were combined and aged under N₂ for 4 h. The reaction was partitioned between sat. NaHCO₃ and ethyl acetate. The layers were separated and the aqueous layer was extracted with ethyl acetate (2×). The combined organics were washed with water and brine then dried over Na₂SO₄, filtered and concentrated under reduced pressure, affording 1.16 g of benzyl 4-[(4-pyridinylamino)carbonyl]-1-piperidinecarboxylate as a yellow oil which was used without further purification.

Step 2:

Benzyl 4-[(4-pyridinylamino)methyl]-1-piperidinecarboxylate

The amide from step 1 above (17.82 g, 52.50 mmol) was dissolved in THF (50 mL) and was treated with BH₃-THF (200 mmol, 200 mL, 1 M in THF) over 10 min. and was aged at r.t. 3 h. The reaction was quenched by slowly adding 2N HCl and stirring vigorously 15 h. The reaction was basified with 1 M NaOH and extracted with ethyl acetate (3×). The combined organics were washed with brine, dried over Na₂SO₄, filtered and concentrated in vacuo, yielding a white foam which was purified by silica gel chromatography (99:1:0.1 to 90:10:1 CH₂Cl₂:CH₃OH:NH₄OH) to give 11.53 g of benzyl 4-[(4-pyridinylamino)methyl]-1-piperidinecarboxylate as a viscous pale yellow oil.

¹H NMR (HCl salt 400 MHz, CD₃OD): δ 8.09 (brs, 1H, Pyr-H), 7.97 (brs, 1H, Pyr-H), 7.35-7.28 (m, 5H, Ar—H), 6.88 (brs, 2H, Pyr-H), 5.11 (s, 2H, CH₂—Ar), 4.18 (brd, J=11.70 Hz, 2H, CHH), 3.25 (d, J=6.77 Hz, 2H, CH₂—N), 2.86 (brs, 2H, CHH), 1.90-1.77 (m, 3H, CHH, CH), 1.29-1.16 (dq, J=12.36 Hz, 4.16 Hz, 2H, CHH).

M.S. (M+1): 326.47.

### Example A2

4-[(3-Methylpyridin-4-ylamino)methyl]piperidine-1-carboxylic acid benzyl ester:

The title compound was prepared as described in EXAMPLE A1, but replacing 4-aminopyridine with 4-amino-3-methylpyridine (Malinowski et al., J. Prakt. Chem., 330:154-158(1988)).

¹H NMR (400 MHz, CD₃OD): δ 7.74 (d, J=5.85 Hz, 1H, Pyr-H), 7.66 (brs, 1H, Pyr-H), 7.36-7.29 (m, 5H, Ar—H), 6.77 (brs, 1H, Pyr-H), 5.11 (s, 2H, CH₂—Ar), 4.19 (brd, J=13.81 Hz, 3H), 3.31-3.20 (m, 2H, CH₂—N+CH₃OH), 2.84 (brs, 2H, CHH), 2.22 (brs, 2H, CHH), 1.98-1.85 (m, 1H, CH), 1.82 (brd, J=12.89 Hz, 2H, CHH), 1.22-1.14 (m, 2H, CHH).

M.S. (M+1): 340.27.

### Example A3

Benzyl 4-{[(2-pyridinyl)amino]methyl}-1-piperidinecarboxylate

The title compound was prepared as described in EXAMPLE A1, but replacing 4-aminopyridine with 2-aminopyridine.

¹H NMR (400 MHz, CD₃OD): δ 10.00 (brs, 1H, NH), 7.82-7.75 (m, 2H, Pyr-H, Pyr-H), 7.38-7.30 (m, 5H, Ar—H), 6.76-6.70 (m, 2H, Pyr-H, Pyr-H), 5.12 (s, 2H, CH₂—Ar), 4.24 (brs, 2H, CHH), 3.16 (brs, 2H, CH₂—N), 2.84 (brs, 2H, CHH), 2.01-1.80 (m, 3H, CH, CHH+H₂O), 1.26-1.18 (m, 2H, CHH).

M.S. (M+1): 326.28.

### Example A4

Benzyl 4-{[(3-pyridinyl)amino]methyl}-1-piperidinecarboxylate

The title compound was prepared as described in EXAMPLE A1, but replacing 4-aminopyridine with 3-aminopyridine.

¹H NMR (500 MHz, CD₃OD): δ 8.01 (d, J=2.93 Hz, 1H, Pyr-H), 7.95 (dd, J=4.63 Hz, 1.46 Hz, 1H, Pyr-1H), 7.37-7.30 (m, 5H, Ar—H), 7.08 (dd, J=8.30 Hz, 4.59 Hz, 1H, Pyr-H), 6.86-6.84 (m, 1H, Pyr-H), 5.13 (s, 2H, CH₂—Ar), 4.25 (brs, 2H, CHH), 3.80 (brt, J=5.86 Hz, 1H, NH), 3.04 (t, J=6.33 Hz, 2H, CH₂—N), 2.78 (brs, 2H, CHH), 1.78 (brs, 3H, CH, CHH+H₂O), 1.27-1.13 (m, 2H, CHH).

M.S. (M+1): 326.31.

### Example A5

Benzyl 4-{[(4-methyl-2-pyridinyl)amino]methyl}-1-piperidinecarboxylate

The title compound was prepared as described in EXAMPLE A1, but replacing 4-aminopyridine with 2-amino-4-methylpyridine.

M.S. (M+1): 340.40.

### Example A6

Benzyl 4-{[(4-ethyl-2-pyridinyl)amino]methyl}-1-piperidinecarboxylate

The title compound was prepared as described in EXAMPLE A1, but replacing 4-aminopyridine with 2-amino-4-ethylpyridine.

M.S. (M+1): 354.41.

### Example A7

Benzyl 4-[(3-isoxazolylamino)methyl]-1-piperidinecarboxylate

The title compound was prepared as described in EXAMPLE A1, but replacing 4-aminopyridine with 3-aminoisoxazole.

M.S. (M+1): 316.29.

### Example A8

Benzyl 4-[(1,3,4-thiadiazol-2-ylamino)methyl]-1-piperidinecarboxylate

The title compound was prepared as described in EXAMPLE A1, but replacing 4-aminopyridine with 2-amino-1,3,4-thiadiazole.

M.S. (M+1): 333.35.

### Example A9

Benzyl 4-{[(5-methyl-2-pyridinyl)amino]methyl}-1-piperidinecarboxylate

The title compound was prepared as described in EXAMPLE A1, but replacing 4-aminopyridine with 2-amino-5-methylpyridine.

M.S. (M+1): 340.40.

### Example A10

Benzyl 4-{[(1-methyl-1H-imidazol-2-yl)amino]methyl)-1-piperidinecarboxylate

The title compound was prepared as described in EXAMPLE A1, step 1, but replacing 4-aminopyridine with 2-amino-imdazole hemisulfate and gave the EDC coupling product. This product was refluxed in DMF-DMA for 90 min., diluted with ethyl acetate, washed with sat. NaHCO₃, dried over Na₂SO₄, filtered and then concentrated under reduced pressure. The resulting red oil was purified by silica gel chromatography. 50 mg (mmol) of the purified product was reacted with borane as described in EXAMPLE A1, step 2, to give 26 mg of benzyl 4-{[(1-methyl-1H-imidazol-2-yl)amino]methyl}-1-piperidinecarboxylate.

1H NMR (400 MHz, CDCl₃): δ 7.36-7.27 (m, 5H, Ar—H), 6.65 (d, J=1.55 Hz, 1H, imidazole-H), 6.49 (d, J=1.56 Hz, 1H, imidazole-H), 5.12 (s, 2H, CH₂—Ar), 4.19 (brs, 2H, CHH), 3.58 (brs, 1H, NH), 3.34 (s, 3H, CH₃), 3.23 (m, 2H, CH₂—N), 2.79 (brs, 2H, CHH), 1.85-1.70 (m, 3H, CHH, CH), 1.23-1.13 (m, 2H, CHH).

M.S. (M+1): 329.27.

### Example A11

### 4-(Quinolin-4-ylaminomethyl)-piperidine-1-carboxylic acid benzyl ester

The title compound was prepared as described in EXAMPLE A1, replacing 4-aminopyridine with 4-aminoquinoline.

M.S. (M+1): 376.39.

### Example A12

Benzyl 4-{[(1-oxido-4-pyridinyl)amino]methyl}-1-piperidinecarboxylate

Step 1:

Benzyl 4-{[(1-oxido-4-pyridinyl)amino]carbonyl}-1-piperidinecarboxylate

Benzyl 4-[(4-pyridinylamino)carbonyl]-1-piperidinecarboxylate (EXAMPLE A1, Step 1) (615 mg, 1.81 mmol) was dissolved in CH₂Cl₂ and treated with mCPBA (3.12 g, 18.10 mmol) and aged 18 h. The reaction was diluted with ethyl acetate and washed with sat. NaHCO₃. The organics were separated, dried over Na₂SO₄, filtered and concentrated under reduced pressure. The resulting oil was purified by silica gel chromatography to afford 124 mg of benzyl 4-{[(1-oxido-4-pyridinyl)amino]carbonyl}-1-piperidinecarboxylate as a clear oil.

¹H NMR (400 MHz, CDCl₃): δ 10.72 (s, 1H, NH), 8.03 (d, J=7.50 Hz, 2H, Pyr-H), 7.80 (d, J=7.50 Hz, 2H, Pyr-H), 7.38-7.28 (m, 5H, Ar—H), 5.12 (s, 2H, CH₂—Ar), 4.18 (brd, J=13.25 Hz, 2H, CHH), 2.81 (brs, 2H, CHH), 2.57-2.45 (m, 1H, CH), 1.86-1.68 (m, 4H, CHH, CHH).

M.S. (M+1): 356.28.

Step 2:

Benzyl 4-{[(1-oxido-4-pyridinyl)amino]methyl}-1-piperidinecarboxylate

Benzyl 4-{[(1-oxido-4-pyridinyl)amino]carbonyl}-1-piperidinecarboxylate (62 mg, 0.17 mmol) was reduced with borane as described in EXAMPLE A1, step 2, to afford 25 mg of benzyl 4-{[(1-oxido-4-pyridinyl)amino]methyl}-1-piperidinecarboxylate as a clear oil.

¹HMR (400 MHz, CDCl₃): δ 7.99 (d, J=7.31 Hz, 2H, Pyr-H), 7.88 (brs, 1H, NH), 7.38-7.30 (m, 5H, Ar—H), 6.66 (brs, 2H, Pyr-H), 5.12 (s, 2H, CH₂—Ar), 4.22 (brs, 2H, CHH), 3.09 (brs, 2H, CH₂—N), 2.77 (brs, 2H, CHH), ), 1.87-1.71 (m, 3H, CHH, CH), 1.26-1.11 (m, 2H, CHH).

M.S. (M+1): 342.33.

### Example A13

Benzyl 4-[(9H-purin-6-ylamino)methyl]-1-piperidinecarboxylate

Step 1:

Benzyl 4-(aminomethyl)piperidine-1-carboxylate

4-Aminomethylpiperidine (40 g, 350 mmol) and benzaldehyde (37.3 mL, 368 mmol) in toluene (600 mL) were heated to reflux under dean stark conditions for 2 h. The resulting reaction mixture was cooled to room temperature and 5001 nL dichloromethane was added. The resulting solution was cooled to 5° C. and treated with N-(benzyloxycarbonyloxy)succinimide (91.7 g, 368 mmol). After 10 min, the cooling bath was removed and the resulting reaction mixture stirred for 1 h. The solvents were evaporated and the residue stirred with 400 mL THF and 400 mL 2M HCl for 1 h. The mixture was concentrated to remove organics and extracted with ether (3×300 mL). The aqueous phase was adjusted to pH14 with 50% NaOH and extracted with ethyl acetate. The organic layer was washed with water and brine, dried over anhydrous sodium sulfate, and the solvent evaporated to give benzyl 4-(aminomethyl)piperidine-1-carboxylate as an oil. (79.7 g)

¹H NMR (500 MHz CDCl₃) δ: 7.4-7.2 (m, 5H); 5.12 (s, 2H); 4.20 (brs, 2H); 2.77 (brs, 2H); 2.58 (d, J=6.6 Hz, 2H) 1.9-1.7 (m, 2H); 1.0-1.5 (m, 5H).

Step 2:

Benzyl 4-[(9H-purin-6-ylamino)methyl]-1-piperidinecarboxylate

In DMF (5 mL), benzyl 4-(aminomethyl)-1-piperidinecarboxylate (1.20 g, 4.83 mmol) and 6-chloropurine (448 mg, 2.49 mmol) were combined and treated with TEA in a single portion and aged under N₂ at 100° C. for 18 h. The resulting reaction was diluted with sat. NaHCO₃ and extracted with ethyl acetate (3×). The combined organics were washed with brine, dried over Na₂SO₄, filtered and concentrated in vacuo to give a brown oil which was purified by silica gel chromatography (20 g, 32-60 um silica, 99:1:0.1 to 90:10:1 CH₂Cl₂:CH₃OH:NH₄OH) to give 600 mg of the benzyl 4-[(9H-purin-6-ylamino)methyl]-1-piperidinecarboxylate as a brown oil.

¹H NMR (400 MHz, CDCl₃): δ 8.42 (s, 1H, purine-H), 7.97 (s, 1H, purine-H), 7.36-7.29 (m, 5H, Ar—H), 6.21 (brs, 1H), 5.13 (s, 2H, CH₂—Ar), 4.22 (brs, 2H, CHH), 3.43 (brs, 2H, CH₂—N), 2.80 (brs, 2H, CHH), 1.95-1.79 (m, 3H, CHH, CH), 1.34-1.21 (m, 2H, CHH).

M.S. (M+1): 367.31.

### Example A14

4-Methylbenzyl 4-[(4-pyrimidinylamino)methyl]-1-piperidinecarboxylate

Step 1:

4-[(2-Methylsulfanyl-pyrimidin-4-ylamino)-methyl]-piperidine-1-carboxylic acid 4-methyl-benzyl ester

The 4-[(2-methylsulfanyl-pyrimidin-4-ylamino)-methyl]-piperidine-1-carboxylic acid 4-methyl-benzyl ester was prepared as described in EXAMPLE A13, Step 2, but replacing 6-chloropurine with 4-chloro-2-methylthiopyrimidine and replacing benzyl 4-(aminomethyl)-1-piperidinecarboxylate with 4-methylbenzyl 4-(aminomethyl)-1-piperidinecarboxylate.

M.S. (M+1): 387

Step 2:

4-Methylbenzyl 4-[(4-pyrimidinylamino)methyl]-1-piperidinecarboxylate

4-[(2-Methylsulfanyl-pyrimidin-4-ylamino)-methyl]-piperidine-1-carboxylic acid 4-methyl-benzyl ester (550 mg, 1.42 mmol)was dissolved in EtOH (15 mL) and treated with Raney Nickel (834 mg, 14.20 mmol) at room temperature for 3 h, filtered, concentrated and purified by silica gel chromatography to give 159 mg of the EXAMPLE A14 as a yellow oil.

¹H NMR (400 MHz, CDCl₃): δ 8.53 (s, 1H, Pyr-1H), 8.13 (brd, J=4.48 Hz, 1H, Pyr-H), 7.24 (d, J=7.86 Hz, 2H, Ar—H), 7.16 (d, J=7.68 Hz, 2H, Ar—H), 6.31 (dd, J=6.00 Hz, 1.20 Hz, 1H, Pyr-H), 5.57 (s, 1H, NH), 5.08 (s, 2H, CH₂—Ar), 4.20 (brs, 2H, CHH), 3.23 (brs, 2H, CH₂—N), 2.75 (brs, 2H, CHH), 2.34 (s, 3H, CH₃), 1.82-1.65 (m, 3H, CHH, CH), 1.23-1.09 (m, 2H, CHH).

M.S. (M+1): 341.35.

### Example A15

Benzyl 4-[(4-pyrimidinylamino)methyl]-1-piperidinecarboxylate

The title compound was prepared as described in EXAMPLE A14, but replacing 4-methylbenzyl 4-(aminomethyl)-1-piperidinecarboxylate with benzyl 4-(aminomethyl)-1-piperidinecarboxylate.

¹H NMR (400 MHz, CDCl₃): δ 8.53 (s, 1H, Pyr-H), 8.13 (brd, J=4.85 Hz, 1H, Pyr-H), 7.38-7.28 (m, 5H, Ar—H), 6.32 (d, J=6.03 Hz, 1H, Pyr-H), 5.51 (brs, 1H, NH), 5.12 (s, 2H, CH₂—Ar), 4.21 (brs, 2H, CHH), 3.24 (brs, 2H, CH₂—N), 2.77 (brs, 2H, CHH), 1.85-1.70 (m, 3H, CHH, CH), 1.27-1.10 (m, 2H, CHH).

M.S. (M+1): 327.29.

### Example A16

Benzyl 4-[(2-pyrimidinylamino)methyl]-1-piperidinecarboxylate

The title compound was prepared as described in EXAMPLE A13, except using benzyl 4-(aminomethyl)-1-piperidinecarboxylate (6.50 g, 26.19 mmol) and 2-chloropyrimidine (990 mg, 8.64 mmol) as starting materials without a solvent to give 1.00 g of the title compound as a yellow oil.

¹H NMR (400 MHz, CDCl₃): δ 8.26 (d, J=4.85 Hz, 1H, Pyr-H), 7.36-7.29 (m, 5H, Ar—H), 6.52 (t, J=4.85 Hz, 1H, Pyr-H), 5.12 (s, 2H, CH₂—Ar), 4.21 (brs, 2H, CHH), 3.30 (t, J=6.26 Hz, 2H, CH₂—N), 2.78 (brs, 2H, CHH), 1.76-1.62 (m, 3H, CHH, CH), 1.28-1.12 (m, 2H, CHH).

M.S. (M+1): 327.33.

### Example A17

4-Methylbenzyl 4-[(2-pyrimidinylamino)methyl]-1-piperidinecarboxylate

The title compound was prepared as described in EXAMPLE A13, except using 4-methylbenzyl 4-(aminomethyl)-1-piperidinecarboxylate (300 mg, 1.14 mmol), 2-chloropyrimidine (131 mg, 1.14 mmol) as starting materials gave 19 mg of the title compound as a yellow oil.

¹H NMR (400 MHz, CDCl₃): δ 8.26 (d, J=4.76, 2H, Pyr-H), 7.26 (d, J=8.96 Hz, 2H, Ar—H), 7.17 (d, J=8.96 Hz, 2H, Ar—H), 6.31 (dd, J=4.85 Hz, 1H, Pyr-H), 5.28 (s, 1H, NH), 5.08 (s, 2H, CH₂—Ar), 4.19 (brs, 2H, CHH), 3.32 (d, J=6.36 Hz, 2H, CH₂—N), 2.76 (brs, 2H, CHH), 2.35 (s, 3H, CH₃), 1.82-1.60 (m, 3H, CHH, CH), 1.25-1.13 (m, 2H, CHH).

M.S. (M+1): 341.37.

### Example A18

Benzyl 4-{[(5-methyl-2-pyrimidinyl)amino]methyl}-1-piperidinecarboxylate

The title compound was prepared as described in EXAMPLE A13, except using benzyl 4-(aminomethyl)-1-piperidinecarboxylate (298 mg, 1.20 mmol), 2-chloro-5-methylpyrimidine (51 mg, 0.40 mmol) as starting materials and using no solvent gave 103 mg of the title compound as a yellow oil.

¹H NMR (400 MHz, CDCl₃): δ 8.10 (s, 2H, Pyr-H), 7.36-7.28 (m, 5H, Ar—H), 5.47 (bt, J=4.98 Hz, 1H, NH), 5.12 (s, 2H, CH₂—Ar), 4.19 (brs, 2H, CHH), 3.32 (d, J=6.22 Hz, 2H, CH₂—N), 2.76 (brs, 2H, CHH), 2.10 (s, 3H, CH₃), 1.82-1.63 (m, 3H, CHH, CH), 1.25-1.12 (m, 2H, CHH).

M.S. (M+1): 341.40.

### Example A19

4-Methylbenzyl 4-({[2-(methylsulfanyl)-4-pyrimidinyl]amino}methyl)-1-piperidinecarboxylate

The title compound was prepared as described in EXAMPLE A13, except using 4-methylbenzyl 4-(aminomethyl)-1-piperidinecarboxylate (600 mg, 2.29 mmol), and 4-chloro-2-methylthiopyrimidine (386 mg, 2.40 mmol) as starting materials and gave 558 mg of the title compound as a yellow oil.

¹H NMR (400 MHz, CDCl₃): δ 7.99 (bs, 1H, Pyr-H), 7.25 (d, J=8.69 Hz, 2H, Ar—H), 7.17 (d, J=8.95 Hz, 2H, Ar—H), 6.00 (d, J=5.94 Hz, 1H, Pyr-H), 5.08 (s, 2H, CH₂—Ar), 4.97 (bs, 1H, NH), 4.21 (brs, 2H, CHH), 3.24 (brs, 2H, CH₂—N), 2.75 (brs, 2H, CHH), 2.48 (s, 3H, CH₃), 2.35 (s, 3H, CH₃), 1.82-1.65 (m, 3H, CHH, CH), 1.27-1.12 (m, 2H, CHH).

M.S. (M+1): 387.34.

### Example A20

Benzyl 4-{[(6-chloro-4-pyrimidinyl)amino}methyl]-1-piperidinecarboxylate

The title compound was prepared as described in EXAMPLE A13, except using 4,6-dichloropyrimidine (1.26 g, 8.45 mmol) in place of 6-chloropurine as starting materials and adding TEA (2.80 mL, 20.13 mmol) in 10 mL DMF. The procedure gave 2.06 g of the title compound as a yellow oil.

¹H NMR (400 MHz, CDCl₃): δ 8.32 (s, 1H, Pyr-H), 7.37-7.28 (m, 5H, Ar—H), 6.35 (s, 1H, Pyr-H), 5.72 (s, 1H, NH), 5.13 (s, 2H, CH₂—Ar), 4.22 (brs, 2H, CHH), 3.23 (brs, 2H, CH₂—N), 2.78 (brs, 2H, CHH), 1.85-1.66 (m, 3H, CHH, CH), 1.27-1.10 (m, 2H, CHH).

M.S. (M+1): 361.32.

### Example A21

Benzyl 4-{[(2-amino-9H-purin-6-yl)amino]methyl}-1-piperidinecarboxylate

The title compound was prepared as described in EXAMPLE A13, except using benzyl 4-(aminomethyl)-1-piperidinecarboxylate (300 mg, 1.21 mmol) and 4-amino-6-chloropurine (68 mg, 0.40 mmol) as starting material. The procedure gave 14 mg of the title compound as a yellow oil.

¹H NMR (400 MHz, CDCl₃): δ 7.60 (s, 1H, purine-H), 7.38-7.28 (m, 5H, Ar—H), 6.01 (vbs, 1H, NH), 5.12 (s, 2H, CH₂—Ar), 4.86 (vbs, 2H, NH₂), 4.19 (brs, 2H, CHH), 3.48 (brs, 2H, CH₂—N), 2.77 (brs, 2H, CHH), 1.88-1.70 (m, 3H, CHH, CH), 1.30-1.13 (m, 2H, CHH).

M.S. (M+1): 382.31.

### Example A22

Benzyl 4-{[(6-chloro-3-pyridazinyl)amino]methyl}-1-piperidinecarboxylate

The title compound was prepared as described in EXAMPLE A13, except using benzyl 4-(aminomethyl)-1-piperidinecarboxylate (1.08 g, 4.34 mmol), 3,6-dichloropyridiazine (636 mg, 4.34 mmol) as starting materials which gave 450 mg of the title compound as a yellow oil.

¹H NMR (400 MHz, CDCl₃): δ7.38-7.28 (m, 6H, Pyr-H, Ar—H), 7.15 (d, J=9.24 Hz, 1H, Pyr-H), 5.12 (s, 2H, CH₂—Ar), 4.89 (bs, 1H, NH), 4.22 (brs, 2H, CHH), 3.32 (brs, 2H, CH₂—N), 2.78 (brs, 2H, CHH), 1.96-1.82 (m, 1H, CH), 1.77 (brd, J=12.34 Hz, 2H, CHH), 1.27-1.12 (m, 2H, CHH).

M.S. (M+1): 361.27.

### Example A23

Benzyl 4-[(3-pyridazinylamino)methyl]-1-piperidinecarboxylate

Benzyl 4-{[(6-chloro-3-pyridazinyl)amino]methyl}-1-piperidinecarboxylate (EXAMPLE A22) (400 mg, 1.11 mmol) was dissolved in abs ethanol. Raney nickel (65 mg, 11.1 mmol) was then added and the resulting reaction was stirred under 1 atm hydrogen for 18 h. The catalyst was filtered and the filtrate was concentrated under reduced pressure. The resulting clear oil was purified by silica gel chromatography to give 9 mg of the title compound as a clear oil.

¹H NMR (400 MHz, CDCl₃): δ 8.54 (dd, J=4.48 Hz, 1.28 Hz, 1H, Pyr-H), 7.38-7.29 (m, 5H, Ar—H), 7.14 (dd, J=9.05 Hz, 4.48 Hz, 1H, Pyr-H), 6.61 (dd, J=8.96 Hz, 1.28 Hz, 1H, Pyr-H), 5.12 (s, 2H, CH₂—Ar), 4.83 (bs, 1H, NH), 4.22 (brs, 2H, CHH), 3.33 (brs, 2H, CH₂—N), 2.78 (brs, 2H, CHH), 1.96-1.71 (m, 3H, CHH, CH), 1.27-1.12 (m, 2H, CHH).

M.S. (M+1): 327.25.

### Example A24

Benzyl 4{[(6-hydroxy-3-pyridazinyl)amino]methyl}-1-piperidinecarboxylate

Benzyl 4-([(6-chloro-3-pyridazinyl)amino]methyl}-1-piperidinecarboxylate (EXAMPLE A22) (37 mg, 0.10 mmol) was dissolved in acetic acid (5 mL) with sodium acetate (82 mg, 1.00 mmol) and was heated to 100° C. for 18 h. The volatiles were removed under reduced pressure and the residue partitioned between sat. NaHCO3 and ethyl acetate. The organics were dried over Na₂SO₄, filtered and concentrated under reduced pressure, affording 35 mg of the title compound as a clear oil.

¹H NMR (400 MHz, CDCl₃): δ 10.78 (brs, 1H, OH), 7.38-7.29 (m, 5H, Ar—H), 6.83 (d, J=10.01 Hz, 1H, Pyr-H), 6.78 (d, J=9.77 Hz, 1H, Pyr-H), 5.12 (s, 2H, CH₂—Ar), 4.20 (brs, 3H, CHH, NH), 3.11 (brs, 2H, CH₂—N), 2.78 (brs, 2H, CHH), 1.87-1.65 (m, 3H, CHH, CH), 1.23-1.13 (m, 2H, CHH).

M.S. (M+1): 343.34.

### Example A25

4-(Pyrazin-2-ylaminomethyl)-piperidine-1-carboxylic acid benzyl ester

Benzyl 4-formyl-1-piperidinecarboxylate (P. E. Maligres, Tetrahedron, 53(32):10983-10992(1997)) (100 mg, 0.40 mmol) and aminopyrazine (46 mg, 0.48 mmol) were dissolved in toluene under N₂ and was heated to reflux under Dean Stark conditions for 18 h. The volatiles were removed in vacuo and the residue taken up in ethanol and treated with solid NaBH₄ (76 mg, 2.00 mmol) in small portions. The reaction aged at 20° C. for 1 h then was quenched with 2N HCl. The reaction was basified with 1M NaOH and was extracted with ethyl acetate (2×). The combined organics were dried over Na₂SO₄, filtered and concentrated in vacuo. The resulting residue was purified by reverse phase HPLC to give 36 mg of the title compound as a yellow oil.

¹H NMR (400 MHz, CD₃OD): δ 8.08 (d, J=1.01 Hz, 1H, Pyr-H), 7.95 (dd, J=3.29 Hz, 1.37 Hz, 1H, Pyr-H), 7.71 (d, J=3.29 Hz, 1H, Pyr-H), 7.35-7.28 (m, 5H, Ar—H), 5.10 (s, 2H, CH₂—Ar), 4.18-4.14 (m, 2H, CHH), 3.27 (d, J=2.14 Hz, 2H, CH₂—N), 2.83 (brs, 2H, CHH), 1.88-1.65 (m, 3H, CHH, CH), 1.23-1.09 (m, 2H, CHH).

M.S. (M+1): 327.26.

### Example A26

Benzyl 4-[(1,3-thiazol-2-ylamino)methyl]-1-piperidinecarboxylate

The title compound was prepared as described in EXAMPLE A25, except using benzyl 4-formyl-1-piperidinecarboxylate (300 mg, 1.21 mmol) and 2-amino-1,3-thiazole (133 mg, 1.33 mmol) as starting materials to give 97 mg of the title compound as a yellow-oil.

¹H NMR (400 MHz, CDCl₃): δ 7.38-7.28 (m, 5H, Ar—H), 7.07 (d, J=3.66 Hz, 1H, thiazole-H), 6.45 (d, J=3.66 Hz, 1H, thiazole-H), 6.39 (brs, 1H, NH), 5.12 (s, 2H, CH₂—Ar), 4.20 (brs, 2H, CHH), 3.15 (d, J=6.58 Hz, 2H, CH₂—N), 2.77 (brs, 2H, CHH), 1.89-1.71 (m, 3H, CHH, CH), 1.26-1.10 (m, 2H, CHH).

M.S. (M+1): 332.34.

### Example A27

4-Methylbenzyl 4-{[(3-methyl-2-pyridinyl)amino]methyl) piperidinecarboxylate

Step 1:

Benzyl 4-{[(3-methyl-2-pyridinyl)amino]carbonyl}-1-piperidinecarboxylate

The benzyl 4-{[(3-methyl-2-pyridinyl)amino]carbonyl}-1-piperidinecarboxylate was prepared as described in EXAMPLE A1, except that 1-[(benzyloxy)carbonyl]-4-piperidinecarboxylic acid (5.00 g, 18.99 mmol), 2-amino-3-methylpyridine (2.16 g, 19.94 mmol), EDC (4.37 g, 22.79 mmol), and HOAt (2.71 g, 19.94 mmol) and DMF (3 mL) were used as starting materials. 5.8 μg of benzyl 4-{[(3-methyl-2-pyridinyl)amino]carbonyl}-1-piperidinecarboxylate was isolated as an off-white solid and used without further purification.

Step 2:

Piperidine-4-carboxylic acid (3-methyl-pyridin-2-yl)-amide

The benzyl 4-{[(3-methyl-2-pyridinyl)amino]carbonyl}-1-piperidinecarboxylate from Step 1 above (5.45 g, 15.42 mmol) was suspended in abs. ethanol (250 mL) and was treated with 10% palladium on carbon (1.50 g) and stirred vigorously for 18 h under 1 atm of hydrogen. The catalyst was filtered off and the filtrate was concentrated under reduced pressure giving 3.61 g of the piperidine-4 carboxylic acid (3-methyl-pyridin-2-yl)-amide as yellow oil.

Step 3:

4-(3-Methyl-pyridin-2-ylcarbamoyl)-piperidine-1-carboxylic acid 4-methyl-benzyl ester

The piperidine-4-carboxylic acid (3-methyl-pyridin-2-yl)-amide from Step 2 above (10 mg, 0.46 mmol) and N-[4-(methylbenzyloxy)-carbonyloxy]succinimide (127 mg, 0.48 mmol) were combined in DMF at r.t. and were mixed vigorously for 15 min. The entire reaction was then purified by preparatory HPLC to give 70 mg of the 4-(3-methyl-pyridin-2-ylcarbamoyl)-piperidine-1-carboxylic acid 4-methyl-benzyl ester as a clear oil.

Step 4:

4-[(3-Methyl-pyridin-2-ylamino)-methyl]-piperidine-1-carboxylic acid 4-methyl-benzyl ester

The 4-(3-methyl-pyridin-2-ylcarbamoyl)-piperidine-1-carboxylic acid 4-methyl-benzyl ester from Step 3 above (65 mg, 0.18 mmol) was treated with 1M BH₃-THF (1.80 mmol, 1.80 mL, 1M in THF) over 10 min. and was aged at r.t. 4 h. The reaction was quenched by slowly adding 2N HCl and stirring vigorously for 30 min. The reaction was basified with sat. NaHCO₃ and extracted with ethyl acetate (2×). The combined organics were washed with brine, dried over Na₂SO₄, filtered and concentrated in vacuo, yielding a white foam which was purified by silica gel chromatography (99:10.1 to 95:5:0.5 CH₂Cl₂:CH₃OH:NH₄OH) to give 62 mg of the 4-[(3-methyl-pyridin-2-ylamino)-methyl]-piperidine-1-carboxylic acid 4-methyl-benzyl ester (alternatively named 4-methylbenzyl 4-{[(3-methyl-2-pyridinyl)amino]methyl}-1-piperidinecarboxylate) as a yellow oil.

¹H NMR (400 MHz, CD₃OD): δ 8.00 (d, J=2.47 Hz, 1H, Pyr-H), 7.26-7.15 (m, 6H, Pyr-H, Ar—H), 6.88 (dd, J=7.03 Hz, 5.12 Hz, 1H, Pyr-H), 5.08 (s, 2H, CH₂—Ar), 4.18 (brs, 2H, CHH), 3.39 (brs, 2H, CH₂—N), 2.78 (brs, 2H, CHH), 2.35 (s, 3H, CH₃), 2.07 (s, 3H, CH₃), 1.90-1.60 (m, 3H, CHH, CH), 1.30-1.10 (m, 4.16 Hz, 2H, CHH).

M.S. (M+1): 354.41.

### Example A28

4-Fluorobenzyl 4-{[(3-methyl-2-pyridinyl)amino]methyl}-1-piperidinecarboxylate

The piperidine compound (600 mg, 2.74 mmol) from EXAMPLE A27, Step 2, was treated in accordance with Steps 3 and 4 of that EXAMPLE A27, except that N-[4-(fluorobenzyloxy)-carbonyloxy]succinimide (805 mg, 3.01 mmol) was used instead of N-[4-(methylbenzyloxy)-carbonyloxy]succinimide in Step 3 to give 481 mg of the 4-fluorobenzyl 4-{[(3-methyl-2-pyridinyl)amino]methyl}-1-piperidinecarboxylate as a clear oil.

¹H NMR (400 MHz, CDCl₃): δ 7.99 (d, J=4.29 Hz, 1H, Pyr-H), 7.34-7.31 (m, 2H, Ar—H), 7.20-7.18 (m, 1H, Pyr-H), 7.05-7.00 (m, 1H, Pyr-H), 6.50 (dd, J=7.13 Hz, 5.12 Hz, 2H, Ar—H), 5.08 (s, 2H, CH₂—Ar), 4.22 (brs, 3H, CHH, NH), 3.38 (brs, 2H, CH₂—N), 2.77 (brs, 2H, CHH), 2.06 (s, 3H, CH₃), 1.84-1.77 (m, 3H, CHH, CH), 1.26-1.12 (m, 2H CHH).

M.S. (M+1): 358.35.

### Example A29

4-Chlorobenzyl 4-{[(3-methyl-2-pyridinyl)amino]methyl}-1-piperidinecarboxylate

The piperidine compound (600 mg, 2.74 mmol) from Example A27, Step 2, was treated in accordance with Steps 3 and 4, except that N-[4-(chlorobenzyl-oxy)carbonyloxy]succinimide (855 mg, 3.01 mmol) was used instead of N-[4-(methylbenzyloxy)-carbonyloxy]succinimide in Step 3 to give 102 mg of the 4-chlorobenzyl 4-{[(3-methyl-2-pyridinyl)amino]methyl}-1-piperidinecarboxylate as a clear oil.

¹H NMR (400 MHz, CDCl₃): δ 7.99 (dd, J=4.90 Hz, 1.23 Hz, 1H, Pyr-H), 7.32-7.27 (m, 4H, Ar—H), 7.20-7.18 (m, 1H, Pyr-H), 6.50 (dd, J=7.18 Hz, 5.08 Hz, 1H, Pyr-H), 5.08 (s, 2H, CH₂—Ar), 4.20 (brs, 3H, CHH, NH), 3.38 (brs, 2H, CH₂—N), 2.78 (brs, 2H, CHH), 2.06 (s, 3H, CH₃), 1.90-1.72 (m, 3H, CHH, CH), 1.26-1.12 (m, 2H CHH).

M.S. (M+1): 374.31.

### Example A30

3-Fluorobenzyl 4-[(4-pyridinylamino)methyl]-1-piperidinecarboxylate

Step 1:

N-(4-piperidinylmethyl)-4-pyridinamine

Benzyl 4-[(4-pyridinylamino)methyl]-1-piperidinecarboxylate (EXAMPLE A1) (7 g, 21 mmol) was dissolved in abs. Ethanol (150 mL) with 10% palladium on carbon (700 mg) and stirred under 1 atm of hydrogen for 2 h. The catalyst was filtered off and the filtrate was concentrated under reduced pressure to afford the N-(4-piperidinylmethyl)-4-pyridinamine as a clear oil which was used without further purification.

Step 2:

3-Fluorobenzyl 4-[(4-pyridinylamino)methyl]-1-piperidinecarboxylate

3-Fluorobenzyl alcohol (30 mg, 0.24 mmol) was treated with triphosgene (24 mg, 0.08 mmol) and N-(4-piperidinylmethyl)-4-pyridinamine (50 mg, 0.26 mmol), and aged at 40° C. for 45 min. The resulting reaction solution was partitioned between 0.5M NaOH and ethyl acetate. The organics were separated, dried over Na₂SO₄, filtered and concentrated under reduced pressure. The resulting oil was purified by preparatory HPLC to give 14 mg of TFA salt of the 3-fluorobenzyl 4-[(4-pyridinylamino)methyl]-1-piperidinecarboxylate as a yellow oil.

M.S. (M+1): 344.36.

The following EXAMPLES A32-A36 were prepared as described above in EXAMPLE A30, but replacing 3-fluorobenzyl alcohol with the appropriate alcohol:

### Example A31

2-Methylbenzyl 4-[(4-pyridinylamino)methyl]-1-piperidinecarboxylate

M.S. (M+1): 340.38.

### Example A32

3-Methylbenzyl 4-[(4-pyridinylamino)methyl]-1-piperidinecarboxylate

M.S. (M+1): 340.39.

### Example A33

4-Methylbenzyl 4-[(4-pyridinylamino)methyl]-1-piperidinecarboxylate

M.S. (M+1): 340.29.

### Example A34

2-Methoxybenzyl 4-[(4-pyridinylamino)methyl]-1-piperidinecarboxylate

M.S. (M+1): 356.37.

### Example A35

3-Methoxybenzyl 4-[(4-pyridinylamino)methyl]-1-piperidinecarboxylate

M.S. (M+1): 356.37.

### Example A36

4-Methoxybenzyl 4-[(4-pyridinylamino)methyl]-1-piperidinecarboxylate

M.S. (M+1): 356.36.

### Example A37

4-Fluorobenzyl 4-[(2-pyrimidinylamino)methyl]-1-piperidinecarboxylate

Benzyl 4-[(2-pyrimidinylarmino)methyl]-1-piperidinecarboxylate (EXAMPLE A16) was hydrogenated as described in Example A30, Step 1. Treatment with N-[4-(fluorobenzyloxy)-carbonyloxy]succinimide as described in EXAMPLE A27, Step 3, afforded the 4-fluorobenzyl 4-[(2-pyrimidinylamino)methyl]-1-piperidinecarboxylate.

¹H NMR (400 MHz, CDCl₃): δ 8.26 (d, J=4.89 Hz, 2H, Pyr-H), 7.35-7.27 (m, 2H, Ar—H), 7.05-7.01 (m, 2H, Ar—H), 6.53 (t, J=4.76 Hz, 1H, Pyr-H), 5.45 (brt, J=5.73 Hz, 1H, NH), 5.08 (s, 2H, CH₂—Ar), 4.20 (brd, J=27.6 Hz, 2H, CHH), 3.32 (t, J=6.22 Hz, 2H, CH₂—N), 2.77 (brs, 2H, CHH), 1.83-1.75 (m, 3H, CHH, CH), 1.26-1.15 (m, 2H CHH).

M.S. (M+1): 345.35.

### Example A38

4-Chlorobenzyl 4-[(2-pyrimidinylamino)methyl]-1-piperidinecarboxylate

The title compound was prepared as described in EXAMPLE A37, except replacing N-[4-(fluorobenzyloxy)-carbonyloxy]-succinimide with N-[4-(chlorobenzyloxy)carbonyloxy]-succinimide.

¹H NMR (400 MHz, CDCl₃): δ 8.25 (d, J=4.75 Hz, 2H, Pyr-H), 7.33-7.27 (m, 4H, Ar—H), 6.51 (t, J=4.84 Hz, 1H, Pyr-H), 5.77 (bs, 1H, NH), 5.08 (s, 2H, CH₂—Ar), 4.18 (brs, 2H, CHH), 3.32 (brt, J=6.12 Hz, 2H, CH₂—N), 2.77 (brs, 2H, CHH), 1.84-1.75 (m, 3H, CHH, CH), 1.26-1.12 (m, 2H CHH).

M.S. (M+1): 361.32.

### Example A39

cis 3-Hydroxy-4-(pyridin-4-ylaminomethyl)-piperidine-1-carboxylic acid benzyl ester

Step 1:

1-Benzyl-4-hydroxymethyl-piperidin-3-ol

Sodium borohydride (40 g) was added in portions to a stirred solution of ethyl N-benzyl-3-oxopiperidine-4-carboxylate hydrochloride in methanol (500 mL), over 2 h. Water (300 mL) was added slowly, the mixture stirred for 15 min and then the organics were evaporated. The residue was partitioned between DCM and water (×3), the combined organic layers dried over anhydrous sodium sulfate, and the solvent evaporated to give the 1-benzyl-4-hydroxymethyl-piperidin-3-ol product (9 g) as a cis/trans mixture, which was used in the next step without further purification.

M.S (M+1): 222.

Step 2:

3-Hydroxy-4-hydroxymethyl-piperidine-1-carboxylic acid benzyl ester

A solution of the 1-benzyl-4-hydroxymethyl-piperidin-3-ol from

Step 1 above (13.5 g) in methanol (450 mL) was hydrogenated at 50 psi over 20% palladium hydroxide on charcoal (10 g) for 48 h in three batches. The combined reaction mixtures were filtered and the filtrate evaporated to give an oil. This was dissolved in water (100 mL) and dioxane (100 mL), cooled to 5° C., and benzyl chloroformate (7.8 mL) was added slowly. 1M NaOH was added to maintain a pH of 10-11. After 30 min, the cooling bath was removed and reaction mixture stirred for 30 min. The reaction mixture was concentrated to remove dioxane and the residue extracted with EtOAc (×3). The combined extracts were washed with brine, dried over anhydrous sodium sulfate and solvent evaporated to give a mixture of cis and trans 3-hydroxy-4-hydroxymethyl-piperidine-1-carboxylic acid benzyl ester products. Purification by flash column chromatography (80% EtOAc hexane to 5% MeOH EtOAc) gave 7.65 g of upper Rf cis isomer and 3.2 g lower Rf trans isomer.

M.S (M+1): 266.

Step 3:

Cis 3-Hydroxy-4-(toluene-4 sulfonyloxymethyl)-piperidine-1-carboxylic acid benzyl ester

A solution of the 3-Hydroxy-4-hydroxymethyl-piperidine-1-carboxylic acid benzyl ester from Step 2 above (7.65 g) in chloroform (200 mL) was treated with pyridine (2.6 mL) and 4-toluenesulfonyl chloride (6.05 g) and the reaction mixture heated to 60° C. for 18 h. Additional pyridine (0.85 mL) and 4-toluenesulfonyl chloride (2.0 g) were added to the cooled reaction and heating continued for a further 24 h. The resulting reaction mixture was cooled to room temperature and washed with 10% aqueous citric acid solution and water, dried over anhydrous sodium sulfate and the solvent evaporated to give, after flash column chromatography, the cis 3-Hydroxy-4-(toluene-4-sulfonyloxymethyl)-piperidine-1-carboxylic acid benzyl ester compound (9.12 g).

Step 4:

Cis 4-Aminomethyl-3-hydroxy-piperidine-1-carboxylic acid benzyl ester

A solution of the tosylate compound (6.80 g) from Step 3 above was dissolved in DMF (50 mL) and treated with sodium azide (3.16 g). The reaction mixture was then heated to 50° C. for 48 h, cooled to room temperature and partitioned between dilute aqueous sodium bicarbonate and EtOAc. The organic layer was washed with brine, dried over anhydrous sodium sulfate and solvent evaporated to give the azide (5.23 g) which was dissolved in THF (50 mL) and treated with triphenylphosphine (14.07 g) and water (3.25 mL). The reaction mixture was stirred for 18 h at room temperature, the volatiles evaporated and the residue purified by flash column chromatography (DCM to 80/20/2 DCM MeOH NH₄OH) to give the cis 4-aminomethyl-3-hydroxy-piperidine-1-carboxylic acid benzyl ester compound as an oil (4.38 g).

M.S (M+1): 265.

Step 5:

cis 3-Hydroxy-4-(pyridin-4-ylaminomethyl)-piperidine-1-carboxylic acid benzyl ester

A mixture of the cis 4-aminomethyl-3-hydroxy-piperidine-1-carboxylic acid benzyl ester (245 mg) from Step 4 above, 4-chloropyridine (105 mg) and isopropanol (0.4 mL) was heated to 120° C. in a sealed vial for 24 h, cooled to room temperature and the solvents evaporated. The resulting crude mixture was purified by flash column chromatography (DCM to 80/20/2 DCM MeOH NH4OH) to give impure cis 3-hydroxy-4-(pyridin-4-ylaminomethyl)-piperidine-1-carboxylic acid benzyl ester. This was purified by preparative reverse phase HPLC (95% H₂O 5% MeCN to 100% MeCN both containing 0.1% TFA). Evaporation gave an oil which was partitioned between DCM and aqueous sodium bicarbonate solution. The organic layer was dried over anhydrous sodium sulfate and solvent evaporated to give a white solid (45 mg).

M.S (M+1): 342.

### Example A40

(−)-cis 3-Hydroxy-4-(pyridin-4-ylaminomethyl)-piperidine-1-carboxylic acid benzyl ester and (+)-cis 3-Hydroxy-4-(pyridin-4-ylaminomethyl)-piperidine-1-carboxylic acid benzyl ester The enantiomers of cis 3-hydroxy-4-(pyridin-4-ylaminomethyl)-piperidine-1-carboxylic acid benzyl ester were separated by preparative HPLC on a Chiralpak® AD column, eluting with 70% (0.1% diethylamine in hexane) 30% isopropanol to give the earlier eluting (−) enantiomer followed by the (+)-enantiomer.

### Example A41

cis 3-Hydroxy-4-(pyridin-4-ylaminomethyl)-piperidine-1-carboxylic acid 4-methyl-benzyl ester

Step 1:

3-Hydroxy-4-[(2,3,5,6-tetrachloro-pyridin-4-ylamino)-methyl]-piperidine-1-carboxylic acid benzyl ester 2,3,5,6-tetrachloro-4-nitropyridine (S. M. Roberts et al., J. Chem. Soc. C, 2844-2848(1968)) (1.7 g, 6.5 mmol) was added to a solution of cis 4-aminomethyl-3-hydroxy-piperidine-1-carboxylic acid benzyl ester (1.71 g, 6.49 mmol) and N-methylmorpholine (0.785 mL, 7.15 mmol) in THF (50 mL) at room temperature. The resulting reaction mixture was stirred for 18 h at room temperature then partitioned between EtOAc and water. The organic layer was washed with saturated sodium bicarbonate solution, dried over anhydrous sodium sulfate and the solvent evaporated to give crude product purified by flash column chromatography (20-80% EtOAc hexane) to give 1.64 g of the 3-hydroxy-4-[(2,3,5,6-tetrachloro-pyridin-4-ylamino)-methyl]-piperidine-1-carboxylic acid benzyl ester compound.

M.S (M+1): 478.

Step 2:

4-(Pyridin-4-ylaminomethyl)-piperidin-3-ol

A suspension of the 3-hydroxy-4-[(2,3,5,6-tetrachloro-pyridin-4-ylamino)-methyl]-piperidine-1-carboxylic acid benzyl ester compound from Step 1 above (1.64 g) and potassium carbonate (6 g) in ethanol (200 mL) was hydrogenated at 60 psi over 1 g of 10% palladium on charcoal for 5 h. The reaction mixture was filtered and the solids washed well with ethanol. The filtrate was evaporated, taken up in 40% MeOH DCM and refiltered. The filtrate was evaporated to give 1.07 g of crude 4-(pyridin-4-ylaminomethyl)-piperidin-3-ol product used without further purification in the next step.

Step 3:

cis 3-Hydroxy-4-(pyridin-4-ylaminomethyl)-piperidine-1-carboxylic acid 4-methyl-benzyl ester

A suspension of the 4-(Pyridin-4-ylaminomethyl)-piperidin-3-ol from Step 2 above (0.076 g, 0.367 mmol) was suspended in DMF (1.5 mL) and treated with carbonic acid 2,5-dioxo-pyrrolidin-1-yl ester 4-methyl-benzyl ester (0.097 g, 0.37 mmol) (prepared as described for analogs in Chem. Pharm. Bull., 38(1):110-115(1990)) and the resulting reaction mixture was stirred at room temperature for 5 min. The mixture was then partitioned between dilute sodium carbonate solution and EtOAc. The organic layer was washed with saturated sodium bicarbonate solution and brine, dried over anhydrous sodium sulfate, and the solvent evaporated to give a crude product. Purification by flash column chromatography (DCM to 80/20/2 DCM MeOH NH4 OH) afforded 60 mg of the cis 3-hydroxy-4-(pyridin-4-ylaminomethyl)-piperidine-1-carboxylic acid 4-methyl-benzyl ester compound.

M.S (M+1): 356.

### Example A42

cis 3-Hydroxy-4-(pyridin-4-ylaminomethyl)-piperidine-1-carboxylic acid 4-ethyl-benzyl ester

The title compound was prepared as described in EXAMPLE A41, Step 3, but replacing carbonic acid 2,5-dioxo-pyrrolidin-1-yl ester 4-methyl-benzyl ester with carbonic acid 2,5-dioxo-pyrrolidin-1-yl ester 4-ethyl-benzyl ester.

M.S (M+1): 370

### Example A43

cis 3-Hydroxy-4-(pyridin-2-ylaminomethyl)-piperidine-1-carboxylic acid benzyl ester

A mixture of cis 4-aminomethyl-3-hydroxy-piperidine-1-carboxylic acid benzyl ester (0.1 g, 378 mmol) and 2-fluoropyridine (0.25 mL) was heated to 120° C. for 24 h. The reaction mixture was partitioned between EtOAc and water. The organic layer was washed with brine, dried over anhydrous sodium sulfate, and the solvent evaporated to give a cis 3-Hydroxy-4-(pyridin-2-ylaminomethyl)-piperidine-1-carboxylic acid benzyl ester crude product, which was purified by flash column chromatography (50% EtOAc hexane to 5% MeOH EtOAc.

M.S (M+1): 342

### Example A44

4-[(3-Cyano-pyridin-2-ylamino)-methyl]-piperidine-1-carboxylic acid benzyl ester

A mixture of cis 4-aminomethyl-3-hydroxy-piperidine-1-carboxylic acid benzyl ester (1 g, 4.03 mmol) and 3-cyanopyridine (0.25 g) was heated to 100° C. for 30 min. The reaction mixture was partitioned between EtOAc and pH5.2 citrate buffer. The organic layer was washed with brine, dried over anhydrous sodium sulfate, and the solvent evaporated to give a solid which was stirred with 5 mL ether and 0.5 mL EtOAc for 1 h and filtered to a solid 4-[(3-cyano-pyridin-2-ylamino)-methyl]-piperidine-1-carboxylic acid benzyl ester (415 mg)

M.S (M+1): 351

### Example A45

4-[(3-Chloro-pyridin-2-ylamino)-methyl]-piperidine-1-carboxylic acid benzyl ester

A mixture of cis 4-aminomethyl-3-hydroxy-piperidine-1-carboxylic acid benzyl ester (1 g, 4.03 mmol) and 3-chloropyridine (0.25 g) was heated to 100° C. for 12 h. The reaction mixture was cooled and partitioned between EtOAc and pH5.2 citrate buffer. The organic layer was washed with brine, dried over anhydrous sodium sulfate and the solvent evaporated to give a crude product. Purification by flash column chromatography (5-50% EtOAc hexane) afforded 159 mg of the 4-[(3-Chloro-pyridin-2-ylamino)-methyl]-piperidine-1-carboxylic acid benzyl ester compound.

M.S (M+1): 360.

### Example A46

4-[(3-Trifluoromethyl-pyridin-2-ylamino)-methyl]-piperidine-1-carboxylic acid benzyl ester

A mixture of cis 4-aminomethyl-3-hydroxy-piperidine-1-carboxylic acid benzyl ester (1 g, 4.03 mmol) and 3-trifluoromethylpyridine (0.25 g) was heated to 100° C. for 12 h. The reaction mixture was cooled and partitioned between EtOAc and pH5.2 citrate buffer. The organic layer was washed with brine, dried over anhydrous sodium sulfate, and the solvent evaporated to give a crude product. Purification by flash column chromatography (5-50% EtOAc hexane) afforded 403 mg of the 4-[(3-trifluoromethyl-pyridin-2-ylamino)-methyl]-piperidine-1-carboxylic acid benzyl ester compound.

M.S (M+1): 394.

### Example A47

4-[(3-Chloro-pyrazin-2-ylamino)-methyl]-piperidine-1-carboxylic acid benzyl ester

A mixture of cis 4-aminomethyl-3-hydroxy-piperidine-1-carboxylic acid benzyl ester (1.25 g, 5.04 mmol) and 2,3-dichloropyrazine (0.25 g) was heated to 100° C. for 1 h. The reaction mixture was cooled and partitioned between EtOAc and pH5.2 citrate buffer. The organic layer was washed with brine, dried over anhydrous sodium sulfate, and the solvent evaporated to give a crude product. Purification by flash column chromatography (5-50% EtOAc hexane) afforded 527 mg of the 4-[(3-chloro-pyrazin-2-ylamino)-methyl]-piperidine-1-carboxylic acid benzyl ester compound.

M.S (M+1): 361.

### Example A48

4-[(3-Hydroxy-pyrazin-2-ylamino)-methyl]-piperidine-1-carboxylic acid benzyl ester

Step 1:

3-[(Piperidin-4-ylmethyl)-amino]-pyrazin-2-ol 4-[(3-Chloro-pyrazin-2-ylamino)-methyl]-piperidine-1-carboxylic acid benzyl ester (2.21 g, 6.12 mmol) and 3M HCl (200 mL) was heated to reflux for 18 h, cooled to room temperature and the volatiles evaporated. The residue was azeotroped with ethanol (3×100 mL) and then stirred with 50 mL ether for 1 h, filtered and the solid dried to yield 1.7 g of a cream solid.

Step 2:

4-[(3-Hydroxy-pyrazin-2-ylamino)-methyl]-piperidine-1-carboxylic acid benzyl ester

To a solution of the 3-[(piperidin-4-ylmethyl)-amino]-pyrazin-2-ol from Step 1 above (0.287 g, 1.021 mmol) in DMF (5 mL) was added triethylamine (0.356 mL, 2.55 mmol), followed by N-(benzyloxycarbonyloxy)succinimide (0.305 g, 1.23 mmol). The reaction was stirred at room temperature for 15 min then partitioned between EtOAc and water. The organic layer was washed with water and brine, dried over anhydrous sodium sulfate and the crude product purified by flash column chromatography (50% EtOAc hexane to 5% MeOH EtOAc) to give an oil which solidified on standing (270 mg).

M.S (M+1): 343.24.

### Example A49

4-[(5-Chloro-pyrimidin-4-ylamino)-methyl]-piperidine-1-carboxylic acid benzyl ester

Step 1:

4-[(2,5,6-Trichloro-pyrimidin-4-ylamino)-methyl]-piperidine-1-carboxylic acid benzyl ester

To a solution of 4-aminomethyl-piperidine-1-carboxylic acid benzyl ester and N,N-diisopropylethylamine (2.6 g, 20 mmol) in THF (40 mL) at −78° C. was added a solution of tetrachloropyrimidine (4.4 g, 20 mmol). The cooling bath was removed and the solution was stirred for 45 min. The solution was concentrated and purified by filtering through a pad of silica gel using ether.

Step 2:

4[(5-Chloro-2,6-bis-methylsulfanyl-pyrimidin-4-ylamino)-methyl]-piperidine-1-carboxylic acid benzyl ester

To 4-[(2,5,6-Trichloro-pyrimidin-4-ylamino)-methyl]-piperidine-1-carboxylic acid benzyl ester (1 g, 2.33 mmol) in DMF was added sodium thiomethoxide (0.4 g, 5.8 mmol). The resulting reaction mixture was stirred for 2 h and quenched with aqueous ammonium chloride. The product was extracted with ethyl acetate, dried (Na₂SO₄), concentrated, and purified by silica gel chromatography (ether/hexanes).

Step 3:

4-[(5-Chloro-pyrimidin-4-ylamino)-methyl]-piperidine-1-carboxylic acid benzyl ester

4-[(5-Chloro-2,6-bis-methylsulfanyl-pyrimidin-4-ylamino)-methyl]-piperidine-1-carboxylic acid benzyl ester (1.0 g, 2.2 mmol) was suspended in ethanol (15 mL) (not very soluble in ethanol so enough ethyl acetate was added to make homogeneous) and excess Raney nickel was added. The resulting reaction mixture was stirred overnight. More Raney Nickel was added and the reaction mixture was heated to 80° C. for 3 h. The mixture was filtered and the solids were washed with hot ethanol/ethyl acetate several times. The organics were concentrated and the resulting residue was purified by silica gel chromatography (isopropanol/methylene chloride). The product was dissolved in ether and treated with ethereal HCl (2.2 mmol) to form the HCl salt which was collected by filtration. The resulting 4-[(5-chloro-pyrimidin-4-ylamino)-methyl]-piperidine-1-carboxylic acid benzyl ester hydrochloride salt was collected by filtration as a colorless solid.

¹H NMR (400 MHz, CD₃OD): δ 8.67 (s, 1 h, pyrimidine), 8.45 (s, 1 h, pyrimidine), 7.32 (m, 5 h, Ar), 5.10 (s, 2 h, CHH), 4.15 (d, J=13.0 Hz, 2 h, CHH), 3.58 (d, J=7.2 Hz, 2 h, CHH), 2.83 (m, 2 h, C1H), 1.97 (m, 1 h, CH), 1.74 (d, J=12.0 Hz, 2 h, CHH).

M.S (M+1): 361.3

### Example A50

4-[(2-Hydroxymethyl-pyridin-4-ylamino)-methyl]-piperidine-1-carboxylic acid benzyl ester

Step 1:

Benzyl 4-(aminomethyl)piperidine-1-carboxylate

4-Aminomethylpiperidine (40 g, 350 mmol) and benzaldehyde (37.3 mL, 368 mmol) in toluene (600 mL) were heated to reflux under dean stark conditions for 2 h. The reaction mixture was cooled to room temperature and 500 mL dichloromethane added. The solution was cooled to 5° C. and treated with N-(benzyloxycarbonyloxy)succinimide (91.7 g, 368 mmol). After 10 min., the cooling bath was removed and the reaction mixture stirred for 1 h. The solvents were evaporated and the residue stirred with 400 mL THF and 400 mL 2M HCl for 1 h. The mixture was concentrated to remove organics and extracted with ether (3×300 mL). The aqueous phase was adjusted to pH14 with 50% NaOH and extracted with ethyl acetate. The organic layer was washed with water and brine, dried over anhydrous sodium sulfate, and the solvent evaporated to give the benzyl 4-(aminomethyl)piperidine-1-carboxylate compound. (79.7 g).

Step 2:

4-[(1-Benzyloxycarbonyl-piperidin-4-ylmethyl)-amino]-pyridine-2-carboxylic acid

To a solution of 4-chloropicolinic acid (0.8 gm, 0.0051 mol) in DMSO (4 mL) was added benzyl 4-(aminomethyl)piperidine-1-carboxylate (2.5 gm, 0.010 mol) and the mixture warmed to 14000 for 18 h. The reaction was cooled and diluted with 10% sodium bicarbonate (100 mL) and washed with ether (2×25 mL). The aqueous extract was washed with dichloromethane (3×50 mL) and dichloromethane extract dried over sodium sulfate and concentrated to an oil (2.4 gm). The oil was chromatographed on silica using dichloromethane/methanol/acetic acid/water-90/10/1/1/to give 4-[(1-benzyloxycarbonyl-piperidin-4-ylmethyl)-amino]-pyridine-2-carboxylic acid (0.59 gm, 32%).

¹H NMR 400 MHz (δ, DMSO) δ: 8.98 (s, 1H); 8.2-8.0 (m, 1H); 7.6-7.2 (m, 5H); 7.01(brs, 1H); 5.08(s, 2H); 4.02 (brd, 2H); 2.80 (brs, 2H); 1.8-1.6 (m, 3H); 1.3-1.1 (m, 2H).

M.S.(M+1): 370.

Step 3:

4-[(2-Hydroxymethyl-pyridin-4-ylamino)-methyl]-piperidine-1-carboxylic acid benzyl ester

To a 0° C. solution of 4-[(1-benzyloxycarbonyl-piperidin-4-ylmethyl)-amino]-pyridine-2-carboxylic acid (0.59 gm, 0.0016 mol) in THF (2 mL) under nitrogen was added a solution of 1.0M borane-tetrahydrofuran (6 mL) and the mixture allowed to stir at room temperature for 1 h. The reaction was cooled to 0° C., quenched with 1N HCl (10 ml), concentrated and diluted with 10% aqueous sodium bicarbonate. Extraction with dichloromethane (2×50 mL) and concentration of the organic layer gave 540 mg of crude material. Column chromatography using dichloromethane/methanol/ammonium hydroxide-90/10/2 and crystallization from diethyl ether gave 4-[(2-hydroxymethyl-pyridin-4-ylamino)-methyl]-piperidine-1-carboxylic acid benzyl ester (340 mg).

¹HNMR (400 MHz CDCL3) δ: 8.13 (d, 1H, J=6.8 Hz); 7.5-7.1 (m, 5H); 6.35 (m, 2H); 5.12(s, 2H); 4.61 (s, 2H); 4.20 (brm, 3H); 3.08 (m, 2H); 2.78(m, 2H) 1.8-1.6 (m, 3H); 1.3-1.1 (m, 2H).

M.S.(M+1): 356.

### Example A51

4-[(2-Dimethylaminomethyl-pyridin-4-ylamino)-methyl]-piperidine-1-carboxylic acid benzyl ester

Step 1:

4-[(2-Dimethylcarbamoyl-pyridin-4-ylamino)-methyl]-piperidine-1-carboxylic acid benzyl ester

To a mixture of 4-[(1-Benzyloxycarbonyl-piperidin-4-ylmethyl)-amino]-pyridine-2-carboxylic acid (EXAMPLE A50, Step 2) (50 mg, 0.000135 mol), 1-hydroxybenzotriazole hydrate (31 mg, 0.0002 mol), 2.0M dimethylamine/THF (0.100 mL, 0.0002 mol) and triethylamine (0.048 mL, 0.0002 mol) in DMF (2 mL) was added 1-ethyl-3-(3-dimethylaminopropyl)carbodiimide hydrochloride (39 mg, 0.0002 mol) and the mixture allowed to stir at room temperature for 7 days. The mixture was quenched into water (10 mL) and extracted with ethyl acetate (20 mL). The ethyl acetate extract was washed with 10% aqueous sodium bicarbonate (10 mL), brine (5 mL), dried over sodium sulfate and filtered. The filtrate was concentrated in vacuo and the residue chromatographed (reverse phase C-18 using acetonitrile/0.1% trifluoroacetic acid in water) to give the 4-[(2-dimethylcarbamoyl-pyridin-4-ylamino)-methyl]-piperidine-1-carboxylic acid benzyl ester compound as its trifluoroacetate salt (28 mg).

M.S.(M+1): 397.

Step 2:

4-[(2-Dimethylaminomethyl-pyridin-4-ylamino)-methyl]-piperidine-1-carboxylic acid benzyl ester

To 4-[(2-Dimethylcarbamoyl-pyridin-4-ylamino)-methyl]-piperidine-1-carboxylic acid benzyl ester (28 mg, 0.05 mmol) was added a solution of 1.0M borane-tetrahydrofuran (2 mL). The reaction was stirred at room temperature for 24 h. The reaction was quenched with 1N HCl (2 mL) and concentrated in vacuo to an oil. Reverse phase chromatography (C-18 using acetonitrile/0.1% trifluoroacetic acid in water) gave upon concentration in vacuo the 4-[(2-dimethylaminomethyl-pyridin-4-ylamino)-methyl]-piperidine-1-carboxylic acid benzyl ester (8 mg).

¹H NMR (400 MHz CD₃OD) δ: 8.10 (m, 1H); 7.4-7.2 (m, 5H); 7.2-6.8 (m, 2H); 5.12(s, 2H); 4.41 (s, 211); 4.18 (m, 2H; 3.30(m, 2H); 2.78(m, 211) 1.8-1.6 (m, 3H); 1.3-1.1 (m, 2H).

M.S.(4+1): 383.

### Example A52

4-[(2-Methylaminomethyl-pyridin-4-ylamino)-methyl]-piperidine-1-carboxylic acid benzyl ester

The title compound was prepared in a similar manner to EXAMPLE A51, except replacing dimethylamine with methylamine in Step 1.

M.S.(M+1): 369.

### Example A53

4-[(3-Chloro-pyrazin-2-ylamino)-methyl]-piperidine-1-carboxylic acid 4-fluoro-benzyl ester

Step 1:

4-Fluorobenzyl 4-(aminomethyl)piperidine-1-carboxylate

The 4-Fluorobenzyl 4-(aminomethyl)piperidine-1-carboxylate was prepared as described in EXAMPLE A13, Step 1, except replacing N-(benzyloxycarbonyloxy)succinimide with N-(4-[4-fluorobenzyl]oxycarbonyloxy)succinimide (prepared as previously described for analogs by Chem. Pharm. Bull., 38(1):110-115(1990).

Step 2:

4-[(3-Chloro-pyrazin-2-ylamino)-methyl]-piperidine-1-carboxylic acid 4-fluoro-benzyl ester

To 2,3-dichloropyrazine (0.160 gm, 0.00107 mol) was added 4-fluorobenzyl 4-(aminomethyl)piperidine-1-carboxylate (0.86 gm, 0.00322 mol) and the resulting mixture heated under nitrogen at 110° C. for 30 min. The reaction was cooled, diluted with ethyl acetate (50 mL), and washed with 10% aqueous sodium/citric acid pH=5.2 (3×30 mL), and 10% aqueous sodium bicarbonate (30 mL). The ethyl acetate extract was dried over sodium sulfate, filtered through a pad of silica and concentrated to an oil. Crystallization from ether/hexane gave the 4-[(3-Chloro-pyrazin-2-ylamino)-methyl]-piperidine-1-carboxylic acid 4-fluoro-benzyl ester (0.376 gm).

¹H NMR (400 MHz DMSO d₆) δ: 7.99 (d, 1H, J=2.7 Hz); 7.52(d, 1H, J=2.7 Hz); 7.41 (d, 1H, J=5.7 Hz); 7.39(d, 1H, J=5.7 Hz); 7.19 (m, 2H); 7.16 (m, 1H); 5.03 (s, 2H); 3.97 (m, 2H); 3.25 (m, 2H); 2.75 (m, 2H); 1.9 (m, 1H); 1.7 (m, 2H); 1.1-0.9 (m, 211).

M.S.(M+1): 379.

### Example A54

4-Hydroxy-4-(pyridin-4-ylaminomethyl)-piperidine-1-carboxylic acid benzyl ester*TFA salt

Step 1:

4-Aminomethyl-1-benzyl-piperidin-4-ol

A mixture of 1-benzyl-4-hydroxy-piperidine-4-carbonitrile (5.00 g, 19.78 mmol) and BH₃.THF (59.35 mmol, 59.35 mL, 1M in THF) was heated at 80° C. for 1 h. Cooled to 0° C. and quenched with conc. HCl (20 mL), the reaction solution was then stirred at rt in 1 h. The reaction solution was basified with 10N NaOH to pH8, and extracted with ethyl acetate (3×100 mL). The combined extracts were washed with water (50 mL), brine (30 mL), dried over Na₂SO₄, filtered and concentrated in vacuo to give the 4-aminomethyl-1-benzyl-piperidin-4-ol compound (4.0 μg).

M.S.(M+1):221.31

Step 2:

4-BOC-aminomethyl-1-benzyl-piperidin-4-ol

To a cooled (0° C.), stirred solution of 4-aminomethyl-1-benzyl-piperidin-4-ol (4.00 g, 18.16 mmol) in dry CH₂Cl₂ (40 mL), under N₂ was slowly added BOC₂O (4.36 g, 19.97 mmol) dissolved in dry CH₂Cl₂ (5 mL). The ice bath was removed and the reaction solution allowed to warm to rt over 1 h, then concentrated in vacuo. The residue was purified by silica gel chromatography, 1-10 (10% NH₄OH in MeOH)/99-90 CH₂Cl₂) to give 4-BOC-aminomethyl-1-benzyl-piperidin-4-ol (3.18 g).

M.S.(M+1):321.41

Step 3:

4-BOC-aminomethyl-piperidin-4-ol

A mixture of 4-BOC-aminomethyl-1-benzyl-piperidin-4-ol (0.50 g, 1.56 mmol), Pd(OH)₂ (20% on carbon, 0.05 g) in absolute ethanol (15 mL) was shaken under 60 psi H₂atmosphere for 3 h. Filtered and concentrated, the reaction gave 0.36 g of the 4-BOC-aminomethyl-piperidin-4-ol compound.

M.S.(M+1):231.28

Step 4:

4-BOC-aminomethyl-1-CBZ-piperidin-4-ol

To a cooled (0° C.), stirred solution of 4-BOC-aminomethyl-piperidin-4-ol (0.35 g, 1.52 mmol) in dried CH₂Cl₂ (5 mL), under N₂ was slowly added CBZ-Cl (0.24 mL, 1.67 mmol), followed by triethyl amine (0.42 mL, 3.04 mmol). The ice bath was removed and the reaction solution was stirred to rt in 1 h, then concentrated in vacuo. The residue was purified by silica gel chromatography (10 CH₂Cl₂: 1-20 IPA: 89-10 hexane)) to give the 4-BOC-aminomethyl-1-CBZ-piperidin-4-ol compound (0.53 g). M.S.(M+1):365.39

Step 5

4-aminomethyl-1-CBZ-piperidin-4-ol

To a stirred solution of 4-BOC-aminomethyl-1-CBZ-piperidin-4-ol (0.50 g, 1.37 mmol) in dried CH₂Cl₂ (3 mL) was slowly added trifluoroacetic acid (3 mL). The resulting reaction solution was stirred at rt for 20 min., then concentrated in vacuo. The residue was dissolved in ethyl acetate (100 mL), washed with sat. aq. NaHCO₃ (20 mL), water (20 mL), brine (10 mL), dried over Na₂SO₄, filtered and concentrated to give 4-aminomethyl-1-CBZ-piperidin-4-ol (0.29 g).

M.S.(M+1):265.32

Step 6:

4-Hydroxy-4-(pyridin-4-ylaminomethyl)-piperidine-1-carboxylic acid benzyl ester*TFA salt

A solution of 4-aminomethyl-1-CBZ-piperidin-4-ol (0.10 g, 0.38 mmol), 4-bromo-pyridine (0.06 g, 0.38 mmol) in IPA (2 mL) was heated at 100° C. in a sealed reaction tube for 7 h. Cooled to rt, the reaction mixture was diluted with ethyl acetate (100 mL), washed with sat. aq. NaHCO₃ (20 mL), water (20 mL), brine (10 mL), dried over Na₂SO₄, filtered and concentrated. The residue was purified by reversed phase chromatography to give the 4-Hydroxy-4-(pyridin-4-ylaminomethyl)-piperidine-1-carboxylic acid benzyl ester compound as a TFA salt (0.017 g).

M.S.(M+1):342.35

### Example A55

4-[(3-Bromo-pyridin-4-ylamino)-methyl]-piperidine-1-carboxylic acid benzyl ester

A mixture of benzyl-4-(aminomethyl)piperidine-1-carboxylate (EXAMPLE A13, Step 1, 0.20 g, 0.81 mmol), 3,4-dibromo-pyridine (Chem. Abstracts, 58:5627) (0.19 g, 0.81 mmol) in IPA (0.5 mL) was heated at 100° C. in a sealed reaction tube for 7 h, then concentrated in vacuo. The residue was purified by silica gel chromatography (DCM IPA hexane)) to give 4-[(3-Bromo-pyridin-4-ylamino)-methyl]-piperidine-1-carboxylic acid benzyl ester (0.06 g).

M.S.(M+1):405.27

### Example 56

4-[(3-Fluoro-pyridin-4-ylamino)-methyl-piperidine-1-carboxylic acid benzyl ester TFA salt

A mixture of benzyl-4-(aminomethyl)piperidine-1-carboxylate (EXAMPLE A13, Step 1, 0.20 g, 0.81 mmol), 3-fluoro-4-iodo-pyridine (Tetrahedron, 49:49-64(1993) (0.18 g, 0.81 mmol) in IPA (0.1 mL) was heated at 100° C. in a sealed reaction tube for 100 h, then concentrated in vacuo. The residue was purified by reversed phase chromatography to give 4-[(3-Fluoro-pyridin-4-ylamino)-methyl]-piperidine-1-carboxylic acid benzyl ester as a TFA salt (0.031 g).

M.S.(M+1):344.36

### Example A57

4-[(2-Chloro-6-methyl-pyrimidin-4-ylamino)-methyl]-piperidine-1-carboxylic acid benzyl ester

To a stirred solution of 2,4-dichloro-6-methyl-pyrimidine (3.61 g, 22.15 mmol), triethyl amine (7.02 mL, 50.34 mmol) in DMF (15 mL) was slowly added benzyl-4-(aminomethyl)piperidine-1-carboxylate (EXAMPLE A13, Step 1, 5.00 g, 20.13 mmol). The resulting reaction solution was stirred at rt for 2 h, then diluted with ethyl acetate (400 mL), washed with water (3×30 mL), brine (30 mL), dried over Na₂SO₄, filtered and concentrated. The residue was purified by silica gel chromatography (20-80% ethyl acetate in hexane) to give 4-[(2-Chloro-6-methyl-pyrimidin-4-ylamino)-methyl]-piperidine-1-carboxylic acid benzyl ester (3.66 g).

M.S.(M+1):375.36

### Example A58

4-[(6-Methyl-pyrimidin-4-ylamino)-methyl]-piperidine-1-carboxylic acid benzyl ester

Step 1:

4-[(6-Methyl-pyrimidin-4-ylamino)-methyl]-piperidine

A mixture of 4-[(2-Chloro-6-methyl-pyrimidin-4-ylamino)-methyl]-piperidine-1-carboxylic acid benzyl ester (EXAMPLE A57, 0.50 g, 1.33 mmol), Pd/C (10%, 0.05 g) in absolute ethanol (15 mL) was vigorously stirred under 1 atm H₂ for 6 h. Filtered and concentrated, the reaction gave 0.27 g of the 4-[(6-Methyl-pyrimidin-4-ylamino)-methyl]-piperidine compound.

M.S.(M+1):207.30

Step 2:

4-[(6-Methyl-pyrimidin-4-ylamino)-methyl]-piperidine-1-carboxylic acid benzyl ester

To a stirred solution of 4-[(6-methyl-pyrimidin-4-ylamino)-methyl]-piperidine (0.15 g, 0.73 mmol), in DMF (1 mL) was added carbonic acid benzyl ester 2,5-dioxo-pyrrolidin-1-yl ester (0.18 g, 0.73 mmol). The resulting reaction solution was stirred at rt for 0.5 h, then concentrated in vacuo. The residue was purified by silica gel chromatography (90:10:1 DCM MeOH NH4OH) to give 4-[(6-Methyl-pyrimidin-4-ylamino)-methyl]-piperidine-1-carboxylic acid benzyl ester (0.24 g).

M.S.(M+1):341.37

### Example A59

4-[(2-Chloro-5-methyl-pyrimidin-4-ylamino)-methyl]-piperidine-1-carboxylic acid benzyl ester

To a stirred solution of 2,4-dichloro-5-methyl-pyrimidine (3.61 g, 22.15 mmol), triethylamine (7.02 mL, 50.34 mmol) in DMF (15 mL) was slowly added benzyl-4-(aminomethyl)piperidine-1-carboxylate (EXAMPLE A13, Step 1, 5.00 g, 20.13 mmol). The resulting reaction solution was stirred at rt for 2 h, then diluted with ethyl acetate (400 mL), washed with water (3×30 mL), brine (30 mL), dried over Na₂SO₄, filtered and concentrated. The residue was purified by silica gel chromatography (20-80% ethyl acetate in hexane) to give 4-[(2-Chloro-5-methyl-pyrimidin-4-ylamino)-methyl]-piperidine-1-carboxylic acid benzyl ester (5.13 g).

M.S.(M+1):375.36

### Example A60

4-[(5-Methyl-pyrimidin-4-ylamino)-methyl]-piperidine-1-carboxylic acid benzyl ester

Step 1:

4-[(5-Methyl-pyrimidin-4-ylamino)-methyl]-piperidine

A mixture of 4-[(2-chloro-5-methyl-pyrimidin-4-ylamino)-methyl]-piperidine-1-carboxylic acid benzyl ester (EXAMPLE A59, 2.00 g, 5.34 mmol), Pd/C (10%, 0.20 g) in absolute ethanol (15 mL) was vigorously stirred under 1 atm H₂. Filtered and concentrated, the reaction gave 1.02 g of 4-[(5-Methyl-pyrimidin-4-ylamino)-methyl]-piperidine.

M.S.(M+1):207.29

Step 2:

4-[(5-Methyl-pyrimidin-4-ylamino)-methyl]-piperidine-1-carboxylic acid benzyl ester

To a stirred solution of 4-[(5-methyl-pyrimidin-4-ylamino)-methyl]-piperidine (0.20 g, 0.97 mmol), in DMF (3 mL) was added carbonic acid benzyl ester 2,5-dioxo-pyrrolidin-1-yl ester (0.24 g, 0.97 mmol). The resulting reaction solution was stirred at rt for 0.5 h, then concentrated in vacuo. The residue was purified by silica gel chromatography (1-10 (10% NH₄OH in MeOH)/99-90 CH₂Cl₂) to give the 4-[(5-methyl-pyrimidin-4-ylamino)-methyl]-piperidine-1-carboxylic acid benzyl ester compound (0.19 g, 58%).

¹H NMR (400 MHz, CDCl₃C) δ 8.50 (s, 1 h, Pyr), 7.97 (s, 1 h, Pyr), 7.35 (m, 5 h, Ar), 5.13 (s, 2 h, ArCH₂O), 4.62 (s, 1 h, NH), 4.22 (br s, 2 h, NCH₂CH₂), 3.43 (s, 2 h, NHCH₂CH), 2.79 (br s, 2 h, NCH₂CH₂), 2.02 (s, 3 h, CH₃), 1.86 (m, 1 h, CH), 1.76 (d, J=11.7 Hz, 2 h, CHCH₂CH₂), 1.21 (q, J=9.7 Hz, 2 h, CHCH₂CH₂);

M.S.(M+1):341.39

EXAMPLES A61-A63 were prepared as described above in EXAMPLE A60, but replacing the carbonic acid benzyl ester 2,5-dioxo-pyrrolidin-1-yl ester with the appropriately substituted analog:

### Example A61

4-[(5-Methyl-pyrimidin-4-ylamino)-methyl]-piperidine-1-carboxylic acid-4-methyl-benzyl ester

¹H NMR (400 MHz, CDCl₃) δ 8.49 (s, 1 h, Pyr), 7.97 (s, 1 h, Pyr), 7.25 (d, J=8.5 Hz, 2 h, Ar), 7.16 (d, J=7.9 Hz, 2 h, Ar), 5.08 (s, 2 h, ArCH₂O), 4.62 (s, 1 h, NH), 4.20 (br s, 2 h, NCH₂CH₂), 3.43 (s, 2 h, NHCH₂CH), 2.77 (t, J=11.0 Hz, 2 h, NCH₂CH₂), 2.35 (s, 3 h, PyrCH₃), 2.02 (s, 3 h, ArCH₃), 1.84 (m, 1 h, CH), 1.74 (d, J=9.7 Hz, 2 h, CHCH₂CH₂), 1.20 (q, J=10.6 Hz, 2 h, CHCH₂CH₂);

M.S.(M+1):355.39

### Example A62

4-[(5-Methyl-pyrimidin-4-ylamino)-methyl]-piperidine-1-carboxylic acid-4-chloro-benzyl ester

1H NMR (400 MHz, CDCl₃) δ 8.50 (s, 1 h, Pyr), 7.97 (s, 1 h, Pyr), 7.34-7.26 (m, 4 h, Ar), 5.08 (s, 2 h, ArCH₂O), 4.62 (s, 1 h, NH), 4.20 (br s, 2 h, NCH₂CH₂), 3.43 (s, 2 h, NHCH₂CH), 2.79 (br s, 2 h, NCH₂CH₂), 2.02 (s, 3 h, CH₃), 1.85 (m, 1 h, CH), 1.76 (d, J=12.6 Hz, 2 h, CHCH₂CH₂), 1.20 (q, J=10.0 Hz, 2 h, CHCH₂CH₂);

M.S.(M+1):375.35

### Example A63

4-[(5-Methyl-pyrimidin-4-ylamino)-methyl]-piperidine-1-carboxylic acid-4-fluoro-benzyl ester

¹H NMR (400 MHz, CD₃OD) δ 8.56 (s, 1 h, Pyr), 7.96 (s, 1 h, Pyr), 7.38 (dd, J=5.6 & 5.4 Hz, 2 h, Ar), 7.08 (t, J=8.7 Hz, 2 h, Ar), 5.08 (s, 2 h, ArCH₂O), 4.14 (d, J=13.3 Hz, 2 h, NCH₂CH₂), 6.94 (d, J=6.9 Hz, 2 h, NHCH₂CH), 2.81 (br s, 2 h, NCH₂CH₂), 2.15 (s, 3 h, CH₃), 1.95 (m, 1 h, CH), 1.74 (d, J=11.4 Hz, 2 h, CHCH₂CH₂), 1.17 (q, J=9.2 Hz, 2 h, CHCH₂CH₂);

M.S.(M+1):359.36

### Example A64

4-[2-Amino-6-methyl-pyrimidin-4-ylamino)-methyl]-piperidine-1-carboxylic acid benzyl ester

Step 1:

4-{[2-(2,4-Dimethoxy-benzylamino)-6-methyl-pyrimidin-4-ylamino]-methyl}-piperidine-1-carboxylic acid benzyl ester A stirred solution of 4-[(2-chloro-6-methyl-pyrimidin-4-ylamino)-methyl]-piperidine-1-carboxylic acid benzyl ester (EXAMPLE A57, 0.5 g, 1.33 mmol) in 2,4-dimethoxy-benzylamine (1.00 mL, 6.67 mmol) was heated at 100° C. for 6 h, then cooled to rt and purified by silica gel chromatography 1-10 (10% NH₄OH in MeOH)/99-90 CH₂Cl₂) to give 4-([2-(2,4-Dimethoxy-benzylamino)-6-methyl-pyrimidin-4-ylamino]-methyl}-piperidine-1-carboxylic acid benzyl ester (0.51 g).

M.S.(M+1):506.46

Step 2:

4-[(2-Amino-6-methyl-pyrimidin-4-ylamino)-methyl]-piperidine-1-carboxylic acid benzyl ester

To a stirred solution of the 4-{[2-(2,4-Dimethoxy-benzylamino)-6-methyl-pyrimidin-4-ylamino]-methyl}-piperidine-1-carboxylic acid benzyl ester from Step 1 above (0.4 g, 0.79 mmol) in CH₂Cl₂ (5 mL) was added trifluoro acetic acid (1 mL). The resulting reaction solution was stirred at rt for 1 h, then concentrated in vacuo. The residue was purified by silica gel chromatography (1-10 (10% NH₄OH in MeOH)/99-90 CH₂C₂) to give the 4-[(2-Amino-6-methyl-pyrimidin-4-ylamino)-methyl]-piperidine-1-carboxylic acid benzyl ester compound (0.27 g).

M.S.(M+1):356.36

### Example A65

4-[(5,6-Dichloro-pyridazin-4-ylamino)-methyl]-piperidine-1-carboxylic acid benzyl ester

Step 1:

3,4,5-Trichloro-pyridazine

A stirred solution of 4,5-dichloro-2,3-dihydro-3-pyridazinone (15.00 g, 90.92 mmol) in POCl₃ was refluxed at 125° C. for 1.5 h, then concentrated in vacuo. The residue was dissolved in CH₂Cl₂ (400 mL), washed with water (100 mL), dried over Na₂SO₄, filtered and concentrated to give 3,4,5-Trichloro-pyridazine (16.18 g).

M.S.(M+1): 185.00

Step 2:

4-[(5,6-Dichloro-pyridazin-4-ylamino)-methyl]-piperidine-1-carboxylic acid benzyl ester

To a stirred solution of 3,4,5-trichloro-pyridazine (2.22 g, 12.08 mmol) and DIPEA (4.21 mL, 24.16 mmol) in IPA (25 mL) was added benzyl-4-(aminomethyl)piperidine-1-carboxylate (EXAMPLE A13, step 1, 3.00 g, 12.08 mmol). The resulting reaction solution was stirred at rt for 5 h, then concentrated in vacuo. The residue was dissolved in CH₂Cl₂ (200 mL), washed with water (50 mL), dried over Na₂SO₄, filtered and concentrated. The crude product was purified by silica gel chromatography (1-7 (10% NH₄OH in MeOH)/99-93 CH₂Cl₂) to give 4-[(5,6-Dichloro-pyridazin-4-ylamino)-methyl]-piperidine-1-carboxylic acid benzyl ester (0.98 g).

M.S.(M+1):395.28

### Example A66

4-[(Pyridazin-4-ylamino)-methyl]-piperidine-1-carboxylic acid benzyl ester

Step 1:

4-[(Pyridazin-4-ylamino)-methyl]-piperidine

A mixture of 4-[(5,6-dichloro-pyridazin-4-ylamino)-methyl]-piperidine-1-carboxylic acid benzyl ester (EXAMPLE A65, 2.00 g, 5.06 mmol), Pd/C (10%, 0.20 g) in absolute ethanol (15 mL) was vigorously stirred under 1 atm H₂ provided by a H₂ balloon for 7 h. Filtered and concentrated, the reaction gave 0.88 g (90%) of the 4-[(Pyridazin-4-ylamino)-methyl]-piperidine compound.

M.S.(M+1): 193.25

Step 2:

4-[(Pyridazin-4-ylamino)-methyl]-piperidine-1-carboxylic acid benzyl ester

To a stirred solution of 4-[(pyridazin-4-ylamino)-methyl]-piperidine (0.20 g, 1.04 mmol), in DMF (3 mL) was added carbonic acid benzyl ester 2,5-dioxo-pyrrolidin-1-yl ester (0.26 g, 1.04 mmol). The resulting reaction solution was stirred at rt for 0.5 h, then concentrated in vacuo. The residue was purified by silica gel chromatography (1-7 (10% NH₄OH in MeOH)/99-93 CH₂Cl₂) to give the 4-[(Pyridazin-4-ylamino)-methyl]-piperidine-1-carboxylic acid benzyl ester (0.18 g).

¹H NMR (400 MHz, CDCl₃) δ 8.65 (d, J=6.1 Hz, 1 h, Pyr), 8.57 (d, J=3.1 Hz, 1 h, Pyr), 7.36 (m, 5 h, Ar), 6.46 (dd, J=6.1 & 2.9 Hz, 1 h, Pyr), 5.13 (s, 2 h, ArCH₂O), 4.40 (s, 1 h, NH), 4.25 (br s, 2 h, NCH₂CH₂), 3.10 (t, J=6.0 Hz, 2 h, NHCH₂CH), 2.78 (br s, 2 h, NCH₂CH₂), 1.81 (m, 1 h, CH), 1.77 (d, J=12.5 Hz, 2 h, CHCH₂CH₂), 1.23 (q, J=10.3 Hz, 2 h, CHCH₂CH₂);

M.S.(M+1):327.28

### Example A67

4-[(Pyridazin-4-ylamino)-methyl]-piperidine-1-carboxylic acid-4-fluoro-benzyl ester

To a stirred solution of 4-[(pyridazin-4-ylamino)-methyl]-piperidine (0.20 g, 1.04 mmol, from EXAMPLE A66, Step 1) in DMF (3 mL) was added carbonic acid-4-fluoro-benzyl ester 2,5-dioxo-pyrrolidin-1-yl ester (0.28 g, 1.04 mmol). The resulting reaction solution was stirred at rt for 0.5 h, then concentrated in vacuo. The residue was purified by silica gel chromatography (1-7 (10% NH₄OH in MeOH)/99-93 CH₂Cl₂) to give the 4-[(Pyridazin-4-ylamino)-methyl]-piperidine-1-carboxylic acid-4-fluoro-benzyl ester (0.28 g).

M.S.(M+1):345.29

### Examples A68A and A68B

### Example A68A

### 4-[(6-Chloro-pyridazin-4-ylamino)-methyl]-piperidine-1-carboxylic acid benzyl ester

### Example A68B

### 4-[(5-Chloro-pyridazin-4-ylamino)-methyl]-piperidine-1-carboxylic acid benzyl ester

A mixture of 4-[(5,6-dichloro-pyridazin-4-ylamino)-methyl]-piperidine-1-carboxylic acid benzyl ester (EXAMPLE A65, 0.15 g, 0.38 mmol), washed Raney Nickel (0.15 g), NH₄OH (1 mL) in absolute ethanol (10 mL) was vigorously stirred under 1 atm H₂ for 7 h. The reaction mixture was filtered and concentrated and the residue was purified by silica gel chromatography (1-7 (10% NH₄OH in MeOH)/99-93 CH₂C₂) to give 4-[(6-chloro-pyridazin-4-ylamino)-methyl]-piperidine-1-carboxylic acid benzyl ester (0.005 g, 4%) M.S.(M+1): 361.25 and 4-[(5-chloro-pyridazin-4-ylamino)-methyl]-piperidine-1-carboxylic acid benzyl ester.

M.S.(M+1):361.25 (0.12 g, 9%)

### Example A69

4-[(2-Chloro-5-fluoro-pyrimidin-4-ylamino)-methyl]-piperidine-1-carboxylic acid benzyl ester

Step 1:

2,4-Dichloro-5-fluoro-pyrimidine

A solution of 5-fluoro-uracil (5.00 g, 38.44 mmol) and N,N-dimethylaniline (5 mL) in POCl₃ (20 mL) was refluxed at 125° C. for 1 h. The solution was then concentrated in vacuo. The resulting residue was quenched with water (20 mL) at 0° C., and extracted with ether (3×150 mL). The combined ether layers were washed with water (2×50 mL), sat. aq. NaHCO₃, water (50 mL), dried over Na₂SO₄, filtered and concentrated to give the 2,4-Dichloro-5-fluoro-pyrimidine compound (5.41 g)

Step 2:

4-[(2-Chloro-5-fluoro-pyrimidin-4-ylamino)-methyl]-piperidine-1-carboxylic acid benzyl ester

To a stirred solution of 2,4-dichloro-5-fluoro-pyrimidine (0.67 g, 4.03 mmol) and triethylamine (0.84 mL, 6.04 mmol) in DMF (5 mL) was added benzyl-4-(aminomethyl)piperidine-1-carboxylate (1.00 g, 4.03 mmol). The resulting reaction solution was stirred at rt for 1 h, and concentrated in vacuo. The residue was purified by silica gel chromatography (CH₂Cl₂/IPA/hexanes) to give 4-[(2-Chloro-5-fluoro-pyrimidin-4-ylamino)-methyl]-piperidine-1-carboxylic acid benzyl ester (0.79 g).

M.S.(M+1):379.25

### Example A70

4-[(5-Fluoro-pyrimidin-4-ylamino)-methyl]-piperidine-1-carboxylic acid benzyl ester

A mixture of 4-[(2-Chloro-5-fluoro-pyrimidin-4-ylamino)-methyl]-piperidine-1-carboxylic acid benzyl ester (EXAMPLE A69, 0.15 g, 0.40 mmol), washed Raney-Ni (0.15 g), NH₄OH (1 mL) in absolute ethanol (10 mL) was vigorously stirred under 1 atm H₂ for 2 h. The reaction mixture was filtered and concentrated and the residue was purified by silica gel chromatography (1-10 (10% NH₄OH in MeOH)/99-90 CH₂Cl₂) to give the 4-[(5-Fluoro-pyrimidin-4-ylamino)-methyl]-piperidine-1-carboxylic acid benzyl ester (0.092 g, 68%).

M.S.(M+1):345.28

### Example A71

4-[(5-Fluoro-pyrimidin-2-ylamino)-methyl]-piperidine-1-carboxylic acid benzyl ester

Step 1:

2-Chloro-5-fluoro-pyrimidine

To a refluxing mixture of 2,4-dichloro-5-fluoro-pyrimidine (EXAMPLE A69, step 1, 3.25 g, 19.47 mmol) and zinc (8-30 mesh, 3.82 g, 58.39 mmol) in THF (30 mL) was slowly added acetic acid (1.1 mL, 19.47 mmol). This reaction mixture was refluxed for 7 h, then cooled to rt, filtered and concentrated to give the 2-Chloro-5-fluoro-pyrimidine compound (2.11 g).

Step 2:

4-[(5-Fluoro-pyrimidin-2-ylamino)-methyl]-piperidine-1-carboxylic acid benzyl ester

A solution of benzyl-4-(aminomethyl)piperidine-1-carboxylate (EXAMPLE A13, step 1, 0.10 g, 0.40 mmol), 2-chloro-5-fluoro-pyrimidine (0.053 g, 0.40 mmol) and triethylamine (0.1 mL, 0.81 mmol) in DMF (0.5 mL) was heated at 100° C. for 6 h, then concentrated in vacuo. The residue was purified by silica gel chromatography (10 CH₂Cl₂: 1-20 IPA: 89-70 hexane) to give the 4-[(5-Fluoro-pyrimidin-2-ylamino)-methyl]-piperidine-1-carboxylic acid benzyl ester (0.037 g).

M.S.(M+1):345.29

### Example A72

4-[(5-Fluoro-pyrimidin-2-ylamino)-methyl]-piperidine-1-carboxylic acid-4-methyl-benzyl ester

The solution of (4-methyl-benzyl)-4-(aminomethyl)piperidine-1-carboxylate (INTERMEDIATE A2a) (0.20 g, 0.76 mmol), 2-chloro-5-fluoro-pyrimidine (EXAMPLE A71, Step 1) (00.10 g, 0.76 mmol) and triethylamine (0.21 mL, 1.53 mmol) in DMF (1 mL) was heated at 100° C. for 6 h, then concentrated in vacuo. The residue was purified by silica gel chromatography (10 CH₂Cl₂: 1-10 IPA: 89-80 hexane) to give the 4-[(5-Fluoro-pyrimidin⁻²-ylamino)-methyl]-piperidine-1-carboxylic acid-4-methyl-benzyl ester (0.11 g).

M.S.(M+1):359.33

### Example A73

4-[(5-Fluoro-pyrimidin-2-ylamino)-methyl]-piperidine-1-carboxylic acid-4-cyclopropyl-benzyl ester

Step 1:

4-Cyclopropyl-benzoic acid ethyl ester

Indium trichloride (2.2 g, 10 mmol) and THF (50 mL) were combined under nitrogen and cooled to −7° C. Cyclopropylmagnesium bromide solution (33 mL, 30 mmol, 0.92 M) was added dropwise while maintaining the reaction temperature≦−60° C. After the addition was complete, the reaction was stirred 0.5 h with cooling then 0.5 h with the cooling bath removed. The resulting solution was added via cannula to a refluxing solution of ethyl-4-iodobenzoate (5.5 g, 20 mmol), trans-dichlorobis(triphenylphosphine)palladium(II) (421 mg, 0.60 mmol) and THF (100 mL) under nitrogen. After 24 h, the contents of the reaction flask were cooled and the solvent was removed in vacuo. Water (100 mL) and 5% KHSO₄ were added and the mixture was extracted with CH₂Cl₂ (3×100 mL). The combined organic extracts were washed with brine, dried with Na₂SO₄ and filtered. The filtrate was removed in vacuo and the remaining residue was purified by flash column chromatography (hexane:EtOAc 95:5) to give the 4-Cyclopropyl-benzoic acid ethyl ester as an orange oil (2.7 g).

Step 2:

(4-Cyclopropyl-phenyl)-methanol

4-Cyclopropyl-benzoic acid ethyl ester (2.46 g, 13 mmol), and THF (250 mL) were combined under nitrogen and cooled in an IPA/dry ice bath to −70° C. Lithium aluminum hydride solution (20 mL, 20 mmol, 1.0M) was added dropwise. After 2 h excess lithium aluminum hydride was quenched by adding EtOAc dropwise. The reaction was warmed to 25° C., then the solvent was removed in vacuo. Water (200 mL) and a few drops of HCl(aq, 6N) were added. The mixture was extracted with EtOAc (3×100 mL). The combined organic extracts were washed with brine, dried with NASO₄ and filtered. The filtrate was removed in vacuo and the remaining residue was purified by flash column chromatography (hexane:EtOAc 40:60) to give the (4-Cyclopropyl-phenyl)-methanol as a colorless oil (2.0 g).

Step 3:

Carbonic acid 4-cyclopropyl-benzyl ester 2,5-dioxo-pyrrolidin-1-yl ester

The title compound was prepared from (4-Cyclopropyl-phenyl)-methanol as described for similar compounds previously (Chem. Pharm. Bull., 38(1):110-115(1990)).

Step 4:

4-Aminomethyl-piperidine-1-carboxylic acid 4-cyclopropyl-benzyl ester

The title compound was prepared from carbonic acid 4-cyclopropyl-benzyl ester 2,5-dioxo-pyrrolidin-1-yl ester as described in EXAMPLE A13, Step 1.

Step 5:

4-[(5-Fluoro-pyrimidin-2-ylamino)-methyl]-piperidine-1-carboxylic acid-4-cyclopropyl-benzyl ester

The solution of (4-cyclopropyl-benzyl)-4-(aminomethyl)piperidine-1-carboxylate (00.10 g, 0.35 mmol), 2-chloro-5-fluoro-pyrimidine (EXAMPLE A71, Step 1, 0.046 g, 0.35 mmol) and triethylamine (0.097 mL, 0.69 mmol) in DMF (1 mL) was heated at 100° C. for 6 h, then concentrated in vacuo. The residue was purified by silica gel chromatography (CH₂Cl₂/IPA/hexanes) to give the 4-[(5-Fluoro-pyrimidin-2-ylamino)-methyl]-piperidine-1-carboxylic acid-4-cyclopropyl-benzyl ester (0.073 g).

M.S.(M+1):385.31

### Example A74

4-[(5-Fluoro-pyrimidin-2-ylamino)-methyl]-piperidine-1-carboxylic acid-4-chloro-benzyl ester

The solution of (4-chloro-benzyl)-4-(aminomethyl)piperidine-1-carboxylate (00.10 g, 0.35 mmol), 2-chloro-5-fluoro-pyrimidine (0.047 g, 0.35 mmol) and triethylamine (0.099 mL, 0.71 mmol) in DMF (1 mL) was heated at 100° C. for 6 h, then concentrated in vacuo. The residue was purified by silica gel chromatography (CH₂Cl₂/IPA/hexanes) to give the 4-[(5-Fluoro-pyrimidin-2-ylamino)-methyl]-piperidine-1-carboxylic acid-4-chloro-benzyl ester (0.057 g).

M.S.(M+1):379.26

### Example A75

4-[(5-Fluoro-pyrimidin-2-ylamino)-methyl]-piperidine-1-carboxylic acid-4-fluoro-benzyl ester

A solution of (4-fluoro-benzyl)-4-(aminomethyl)piperidine-1-carboxylate (00.10 g, 0.38 mmol), 2-chloro-5-fluoro-pyrimidine (0.05 g, 0.38 mmol) and triethylamine (0.11 mL, 0.75 mmol) in DMF (1 mL) was heated at 100° C. for 6 h, then concentrated in vacuo. The residue was purified by silica gel chromatography (CH₂Cl₂/IPA/hexanes) to give the 4-[(5-Fluoro-pyrimidin-2-ylamino)-methyl]-piperidine-1-carboxylic acid-4-fluoro-benzyl ester (0.042 g).

M.S.(M+1):363.31

### Example A76

4-Methylbenzyl 4-[(2-pyrimidinylamino)methyl]-1-piperidinecarboxylate

The stirred solution of 4-methylbenzyl 4-(aminomethyl)-1-piperidinecarboxylate (20.00 g, 76.23 mmol), 2-chloro-pyrimidine (8.73 g, 76.23 mmol) and triethylamine (21.25 mL, 152.46 mmol) in DMF (40 mL) was heated at 100° C. for 6 h. The reaction solution was cooled to rt, then diluted with ethyl acetate (800 mL), washed with sat. aq. NaHCO₃ (100 mL), water (3×100 mL), brine (100 mL), dried over Na₂SO₄, filtered and concentrated. The residue was purified by silica gel chromatography (CH₂Cl₂/IPA/hexanes) to give the 4-Methylbenzyl 4-[(2-pyrimidinylamino)methyl]-1-piperidinecarboxylate (20.12).

M.S.(M+1): 341.30

### Example A77

[1-(2-Phenyl-ethanesulfonyl)-piperidin-4-ylmethyl]-pyrimidin-2-yl-amine

Step 1:

4-Aminomethyl-piperidine-1-carboxylic acid tert-butyl ester

To a mixture of 4-aminomethylpiperidine (15 g) in 250 mL of anhydrous tetrahydrofuran cooled to −78° C. was added, dropwise over 45 min., a solution of di-tert-butyl di-carbonate (24 g) in 100 mL of anhydrous tetrahydrofuran. After stirring for 1 h at −78° C., the mixture was allowed to warm to room temperature and stirred overnight. The mixture was concentrated to near dryness and diluted with 200 mL of 10% aqueous citric acid. The mixture was extracted with 3×100 mL of ether, then made basic with sodium hydroxide pellets and extracted with 3×200 mL of chloroform. The combined chloroform extracts were dried over magnesium sulfate and concentrated to dryness under reduced pressure. The resulting oil (25 g) was homogeneous by TLC (development with 90:10 chloroform saturated with ammonia: methanol).

¹H NMR (400 MHz, CDCl₃): δ4.1 (br s, 2H), 2.7 (br m, 2H), 2.6 (d, 2H), 1.7 (m, 3H), 1.42 (s, 9H), 1.1 (m, 2H).

Step 2:

4-(Benzyloxycarbonylamino-methyl)-piperidine-1-carboxylic acid tert-butyl ester

To a solution of 4-aminomethyl-piperidine-1-carboxylic acid tert-butyl ester (21 g) in 100 mL of ethyl acetate cooled to 0° C. was added 100 mL of saturated sodium carbonate and benzyl chloroformate (17 g). The solution was stirred for 3 h, then separated. The organic layer was dried over magnesium sulfate and concentrated under reduced pressure. Drying under vacuum gave 35 g of an oil:

¹H NMR (400 MHz, CDCl₃): δ 7.35 (m, 5H), 5.3 (d, 1H), 5.1 (s, 2H), 4.1 (br s, 2H), 3.0 (br m, 2H), 2.6 (br m, 2H), 1.7 (m, 3H), 1.42 (s, 9H), 1.1 (m, 2H).

Step 3:

Piperidin-4-ylmethyl-carbamic acid benzyl ester

A mixture of 4-(benzyloxycarbonylamino-methyl)-piperidine-1-carboxylic acid tert-butyl ester (35 g) and 5 mL of 4N HCl in dioxane was stirred at room temperature for 3 h, then diluted with 200 mL of ether and filtered. There was obtained 25 g of piperidin-4-ylmethyl-carbamic acid benzyl ester hydrochloride salt as a white fluffy solid. The free base was obtained by partitioning the hydrochloride between 50 mL chloroform and 50 mL saturated aqueous Na₂CO₃.

¹H NMR (400 MHz, CDCl₃)): δ 7.35 (m, 5H), 5.15 (s, 2H), 4.9 (br s, 1H), 3.1 (m, 2H), 2.6 (m, 3H), 1.7 (m, 2H), 1.6 (m, 2H), 1.1 (m, 2H).

MS (m+1)=249.

Step 4:

[1-(2-Phenyl-ethenesulfonyl)-piperidin-4-ylmethyl]-carbamic acid benzyl ester

A mixture of piperidin-4-ylmethyl-carbamic acid benzyl ester hydrochloride (2 g), 25 mL of dichloromethane, trans-2-styrenesulfonyl chloride (1.5 g), and 3 mL of N,N-diisopropylethylamine was stirred at room temperature overnight, then diluted with 200 mL af chloroform and washed with 100 mL of saturated sodium carbonate. The chloroform extracts were dried over magnesium sulfate and concentrated. There was obtained 2.5 g of [1-(2-phenyl-ethenesulfonyl)-piperidin-4-ylmethyl]-carbamic acid benzyl ester as a white solid.

¹H NMR (400 MHz, CDCl₃)): δ 7.5-7.2 (m, 10H), 6.65 (m, 1H), 5.15 (s, 2H), 4.8 (br s, 1H), 3.8 (d, 2H), 3.1 (dd, 2H), 2.6 (dd, 2H), 1.8 (d, 2H), 1.6 (m, 2H), 1.35 (m, 2H)

MS (m+1)=415.

Step 5:

C-[1-(2-Phenyl-ethanesulfonyl)-piperidin-4-yl]-methylamine

A mixture of [1-(2-phenyl-ethenesulfonyl)-piperidin-4-ylmethyl]-carbamic acid benzyl ester (2.5 g), 20% palladium hydroxide (1 g) on carbon, 200 mL of methanol and 5 mL of tetrahydrofuran were shaken under 50 psi of hydrogen for 2 days at room temperature. The catalyst was filtered off and washed with 250 mL of methanol. Concentration under reduced pressure gave 1.5 g of C-[1-(2-phenyl-ethanesulfonyl)-piperidin-4-yl]-methylamine as white solid.

¹H NMR (400 MHz, CDCl₃)): δ 7.4-7.2 (m, 5H), 5.1 (s, 2H), 3.8 (d, 2H), 3.1 (m, 4H), 2.7 (dd, 2H), 1.8 (d, 2H), 1.6 (m, 5H), 1.3 (m, 2H)

MS (m+1)=283.

Step 6:

[1-(2-Phenyl-ethanesulfonyl)-piperidin-4-ylmethyl]-pyrimidin-2-yl-amine

A mixture of 0.5 g of [1-(2-phenyl-ethanesulfonyl)-piperidin-4-ylmethyl]-pyrimidin-2-yl-amine, 0.56 g of 2-bromopyrimidine, 25 mL of 2-propanol and 0.5 mL of N,N-diisopropylethylamine was heated to reflux overnight. Purification of the residue obtained after concentration under reduced pressure by preparative chromatography, and eluting with ethyl acetate gave 0.6 g of [1-(2-phenyl-ethanesulfonyl)-piperidin-4-ylmethyl]-pyrimidin-2-yl-amine as a white solid.

¹H NMR (400 MHz, CDCl₃)): δ 8.15 (d, 2H), 7.3-7.18 (m, 5H), 6.5 (dd, 1H), 5.5 (dd, 1H), 3.8 (d, 2H), 3.35 (d, 2H), 3.15 (dd, 4H), 2.7 (m, 2H), 1.9 (d, 2H), 1.8 (m, 1H), 1.3 (m, 2H)

MS (m+1)=361.

### Example A78

{1-[2-(4-Fluoro-phenyl)-ethanesulfonyl]-piperidin-4-ylmethyl}-pyrimidin-2-yl-amine

Step 1:

1-(2-Chloro-ethyl)-4-fluoro-benzene

A mixture of 7 g 2-(4-fluoro-phenyl)-ethanol, 25 mL of chlorobenzene, 42 mL of 37% HCl, and 0.9 g of Aliquat® 336 (tricaprylylmethyl ammonium chloride) was heated to reflux for 3 days, cooled and extracted into 3×10 mL of hexane. The combined extracts were dried over magnesium sulfate and concentrated under reduced pressure. The resulting oil, 25 g, was mainly 1-(2-chloro-ethyl)₄-fluoro-benzene:

¹H NMR (400 MHz, CDCl₃): δ 7.3 (dd, 2H), 7.0 (dd, 2H), 3.7 (t, 2H), 3.05 (t, 2H).

Step 2:

Thioacetic acid S-[2-(4-fluoro-phenyl)-ethyl]ester

A mixture of 2.4 g of 1-(2-chloro-ethyl)-4-fluoro-benzene, 30 mL of DMF and 25 mL of potassium thioacetate was stirred under nitrogen for 24 h. The mixture was diluted with 200 mL of water and extracted with 3×50 mL of dichloromethane. The combined organic layers were dried over magnesium sulfate and concentrated under reduced pressure. Drying under vacuum gave 2.5 g of an oil:

¹H NMR (400 MHz, CDCl₃): δ 7.18 (dd, 2H), 6.98 (dd, 2H), 3.08 (t, 2H), 2.81 (t, 2H), 2.32 (s, 3H).

Step 3:

2-(4-Fluoro-phenyl)-ethanesulfonyl chloride

A stream of chlorine gas was dispersed into a stirred, ice cold mixture of 2.5 g of thioacetic acid S-[2-(4-fluoro-phenyl)-ethyl]ester, 30 mL of dichloromethane and 30 mL of water over 1 h. The mixture was diluted with 200 mL of dichloromethane, shaken and separated. The combined organic layers were dried over magnesium sulfate and concentrated under reduced pressure. Trituration with hexane gave 2.5 g of a white solid:

¹H NMR (400 MHz, CDCl₃): δ7.2 (dd, 2H), 7.0 (dd, 2H), 3.1 (dd, 2H), 3.3 (dd, 2H), 2.32 (s, 3H).

Step 4:

4-(tert-Butoxycarbonylamino-methyl)-piperidine-1-carboxylic acid benzyl ester

To an ice cold, stirred solution of 21 g of 4-aminomethyl-piperidine-1-carboxylic acid benzyl ester in 250 mL of dichloromethane was added 18 g of di-tert-butyldicarbonate in 100 mL of dichloromethane over 30 min. After stirring overnight, the mixture was concentrated to dryness. Trituration with hexane gave 28 g of a white solid:

¹H NMR (400 MHz, CDCl₃): δ 7.4 (m, 5H), 5.15 (s, 2H), 4.6 (br s, 1H), 4.2 (br s, 2H), 3.0 (br s, 2H), 2.8 ((m, 2H), 1.7 (m, 3H), 1.42 (s, 9H), 1.15 (m, 2H).

Step 5:

Piperidin-4-ylmethyl-carbamic acid tert-butyl ester

A mixture of 28 g of 4-(tert-butoxycarbonylamino-methyl)-piperidine-1-carboxylic acid benzyl ester, 1 g of 10% palladium on carbon, 100 mL of THF and 200 mL of methanol was stirred under anatmosphere of hydrogen for 2 days. The mixture was filtered concentrated under reduced pressure. Drying under reduced pressure gave 17 g of a white solid:

¹H NMR (400 MHz, CDCl₃): δ 4.8 (br s, 1H), 3.05 (d, 2H), 2.9 (dd, 2H), 2.6 (m, 3H), 1.6 (d, 2H), 1.5 (m, 1H), 1.4 (s, 9H), 1.05 (m, 2H).

Step 6:

{1-[2-(4-Fluoro-phenyl)-ethanesulfonyl]-piperidin-4-ylmethyl}-carbamic acid tert-butyl ester

To an ice cold, stirred solution of 0.2 g of piperidin-4-ylmethyl-carbamic acid tert-butyl ester and 0.2 mL of N,N-diisopropylethylamine in 20 mL of dichloromethane was added 0.3 g of 2-(4-fluoro-phenyl)-ethanesulfonyl chloride. After stirring overnight the mixture was diluted with 50 mL of chloroform, washed with 50 mL of saturated sodium carbonate, dried over magnesium sulfate and concentrated to dryness under reduced pressure. Trituration with hexane gave 0.4 g of a white solid:

¹H NMR (400 MHz, CDCl₃): δ 7.2 (m, 2H), 7.0 (dd, 2H), 4.6 (br m, 1H), 3.8 (d, 2H), 3.1 (m, 3H), 3.0 (m, 2H), 2.7 (dd, 2H), 1.8 (d, 2H), 1.6 (br m, 2H), 1.42 (s, 9H), 1.3 (m, 2H).

Step 7:

C-{1-[2-(4-Fluoro-phenyl)-ethanesulfonyl]-piperidin-4-yl}-methylamine

A mixture of 0.4 g of {1-[2-(4-fluoro-phenyl)-ethanesulfonyl]-piperidin-4-ylmethyl}-carbamic acid tert-butyl ester and 51 mL of 4N HCl in dioxane was stirred at room temperature for 3 h, then diluted with 50 mL of chloroform, washed with 50 mL of saturated sodium carbonate, dried over magnesium sulfate and concentrated to dryness under reduced pressure. The product was a white solid:

¹H NMR (400 MHz, CDCl₃): δ 7.2 (m, 2H), 7.0 (dd, 2H), 3.92 (d, 2H), 3.1 (s, 4H), 2.7 (dd, 2H), 2.6 (d, 2H), 1.8 (d, 2H), 1.5 (br m, 3H), 1.3 (m, 2H)

MS (m+1)=301.

Step 8:

{1-[2-(4-Fluoro-phenyl)-ethanesulfonyl]-piperidin-4-ylmethyl}-pyrimidin-2-yl-amine

A mixture of 0.3 g of C-{1-[2-(4-Fluoro-phenyl)-ethanesulfonyl]-piperidin-4-yl}-methylamine, 0.3 g of 2-bromopyrimidine, 25 mL of 2-propanol and 0.3 mL of N,N-diisopropylethylamine was heated to reflux overnight. Purification of the residue obtained after concentration under reduced pressure by preparative chromatography, eluting with ethyl acetate gave 0.6 g of a white solid.

¹H NMR (400 MHz, CDCl₃)): δ 8.25 (d, 2H), 7.2 (m, 2H), 7.0 (dd, 2H), 6.58 (dd, 1H), 5.25 (br m, 1H), 3.82 (d, 2H), 3.4 (dd, 2H), 3.15 (s, 4H), 2.75 (dd, 2H), 1.9 (d, 2H), 1.8 (m, 1H), 1.3 (m, 2H) MS (m+1)=379.

### Example A79

3-(Pyrimidin-2-ylaminomethyl)-pyrrolidine-1-carboxylic acid benzyl ester

Step 1:

1-Benzyl-pyrrolidine-3-carboxylic acid amide

To a mixture of 4.4 g 1-benzyl-pyrrolidine-3-carboxylic acid methyl ester (M. J. Kornet, P. A. Thio, S. E. Tan, J. Organic Chemistry, 33:3637-3639(1968) and 3 g formamide in 10 mL of anhydrous DMF heated to 100° C., a solution of sodium methoxide, from 0.33 g of sodium dissolved in methanol, was added dropwise over 20 minutes. After stirring for 1 h at 100° C., the mixture was allowed to cool to room temperature and added to 100 mL of isopropanol. The mixture was concentrated to dryness. The residue was triturated with 200 mL of chloroform, filtered and concentrated to dryness under reduced pressure. The resulting oil (4.5 g) was fairly homogeneous by TLC (development with 90:10 chloroform saturated with ammonia: methanol):

¹H NMR (400 MHz, CDCl₃): δ 7.1 (5H), 4.3 (br s, 2H), 3.5 (d, 2H), 3.4 (m, 1H), 2.6 (m, 2H), 2.5 (m, 1H), 2.25 (m, 1H), 1.9 (m, 1H).

Step 2:

3-Carbamoyl-pyrrolidine-1-carboxylic acid benzyl ester

A mixture of 4.5 g 1-benzyl-pyrrolidine-3-carboxylic acid amide, 200 mL THF, 20 mL methanol and 1 g 20% palladium hydroxide on carbon was shaken under 50 psi of hydrogen for 12 h. The catalyst was filtered off and the filtrate concentrated under reduced pressure. Drying under vacuum gave 3 g of an oil. To a stirred solution of the crude residue in 500 mL of chloroform was added 5.5 g of N-(benzyloxycarbonyloxy)succinimide and 2.2 mL of triethylamine. The mixture was allowed to stir overnight then washed with 50 mL of saturated sodium carbonate, dried over magnesium sulfate, and concentrated to dryness. Purification by chromatography on silica gel, eluting with 90:10 ethyl acetate: methanol, gave 1.1 g of 3-Carbamoyl-pyrrolidine-1-carboxylic acid benzyl ester as a resin:

¹H NMR (400 MHz, CDCl₃): δ 7.35 (m, 5H), 5.6 (br m, 2H), 3.6 (m, 3H), 3.4 (m, 1H), 2.9 (br m, 1H), 2.1 (m, 2H).

Step 3:

3-Aminomethyl-pyrrolidine-1-carboxylic acid benzyl ester

A mixture of 1 g 3-carbamoyl-pyrrolidine-1-carboxylic acid benzyl ester and 24 mL 1M borane-THF was stirred at room temperature for 24 h, then carefully quenched with 50 mL of 3N HCl. The mixture was concentrated under reduced pressure, then partitioned between 50 mL chloroform and 25 mL saturated aqueous sodium carbonate. Concentration of the combined extracts after drying over magnesium sulfate gave 0.89 g of 3-Aminomethyl-pyrrolidine-1-carboxylic acid benzyl ester as a resin:

¹H NMR (400 MHz, CDCl₃)): δ 7.35 (m, 5H), 5.15 (s, 2H), 3.7-4 (complex, 4H), 2.7 (m, 1H), 2.4-2.0 (complex, 2H), 1.6 (m, 4H).

Step 4:

3-(Pyrimidin-2-ylaminomethyl)-pyrrolidine-1-carboxylic acid benzyl ester

A mixture of 3-aminomethyl-pyrrolidine-1-carboxylic acid benzyl ester (0.15 g), 2-bromopyrimidine (0.25 g), 2-propanol (10 mL), and of N,N-diisopropylethylamine (0.1 mL) was heated to reflux overnight. Purification of the residue obtained after concentration under reduced pressure by preparative chromatography, and eluting with ethyl acetate, gave 0.2 g of 3-(pyrimidin-2-ylaminomethyl)-pyrrolidine-1-carboxylic acid benzyl ester as a solid:

¹H NMR (400 MHz, CDCl₃)): δ 8.15 (d, 2H), 7.3 (m, 5H), 6.5 (dd, 1H), 5.8 (m, 1H), 5.1 (s 2H), 3.s (m, 2H), 3.4 (m, 3H), 3.2 (m, 1H), 2.55 (m, 1H), 2.0 (m, 1H), 1.7 (m, 1H)

MS (m+1)=313.

### Example A80

(R,S) 4-[1-(Pyridin-4-ylamino)-ethyl]-piperidine-1-carboxylic acid benzyl ester

Step 1:

4-Acetyl-piperidine-1-carboxylic acid benzyl ester

To a solution of 5 g of 4-(N-methoxy-N-methyl-carbamoyl)-piperidine-1-carboxylic acid benzyl ester (S. Nahm and S. W. Weinreb, Tetrahedron Letters, 22:3815-3818(1981)) in 50 mL of anhydrous THF cooled to 0° C., was added dropwise 6 mL of 3M methylmagnesium bromide in ether over 10 minutes. After stirring for 1 h at 0° C., the resulting mixture was quenched with 50 mL of 1N HCl and extracted with 3×50 mL of ether. The combined extracts were dried over magnesium sulfate and concentrated to dryness under reduced pressure. Drying under vacuum gave 4.2 g of 4-Acetyl-piperidine-1-carboxylic acid benzyl ester as a white solid:

1H NMR (400 MHz, CDCl₃): δ 7.35 (m, 5H), 5.15 (s, 2H), 4.2 (br s, 2H), 2.9 (br t, 2H), 2.5 (m, 1H), 2.2 (s, 3H), 1.9 (m, 2H), 1.6 (m, 2H).

Step 2:

(R,S) 4-(1-Hydroxyimino-ethyl)-piperidine-1-carboxylic acid benzyl ester

A mixture of 4.0 g of 4-acetyl-piperidine-1-carboxylic acid benzyl ester, 25 mL of pyridine, and 6 g of hydroxylamine hydrochloride were heated to 100° C. for 12 h. The mixture was concentrated under reduced pressure and partitioned between 200 mL of ethyl acetate and 50 mL of 1N HCl. The organic extract was dried over magnesium sulfate and concentrated to dryness under reduced pressure. Drying under vacuum gave 5 g of (R,S) 4-(1-Hydroxyimino-ethyl)-piperidine-1-carboxylic acid benzyl ester as a solid:

¹H NMR (400 MHz, CDCl₃): δ 7.35 (m, 5H), 5.15 (s, 2H), 4.3 (br s, 2H), 2.8 (br t, 2H), 2.3 (m, 1H), 2.05 and 1.85 (2s, 3H), 1.8 (m, 2H), 1.5 (m, 2H).

Step 3:

(R,S) 4-(1-Hydroxyimino-ethyl)-piperidine-1-carboxylic acid tert-butyl ester

A mixture of 3.2 g of 4-(1-hydroxyimino-ethyl)-piperidine-1-carboxylic acid benzyl ester, 0.4 g of di-tert-butyldicarbonate, 0.15 g of 10% palladium on carbon and 20 mL of THF was stirred under anatmosphere of hydrogen for 2 h. The mixture was filtered and concentrated under reduced pressure. Drying under vacuum gave 3.5 g of a (R,S) 4-(1-Hydroxyimino-ethyl)-piperidine-1-carboxylic acid tert-butyl ester resin:

¹H NMR (400 MHz, CDCl₃): δ 4.15 (br s, 2H), 2.7 (br t, 2H), 2.25 (m, 1H), 1.8 (s, 3H), 1.7 (m, 2H), 1.42 (m, 2H), 1.4 (s, 9H).

Step 4:

(R,S) 4-(1-Amino-ethyl)-piperidine-1-carboxylic acid tert-butyl ester

A mixture of 3 g of 4-(1-hydroxyimino-ethyl)-piperidine-1-carboxylic acid tert-butyl ester, 5 g of wet Raney-nickel and 100 mL of 5% ammonia in ethanol was shaken under 55 psi of hydrogen for 12 h. The mixture was filtered and concentrated under reduced pressure. The resulting crude product was taken up in 250 mL of chloroform, dried over magnesium sulfate, and concentrated under reduced pressure. Drying under vacuum gave 3.5 g of a (R,S) 4-(1-Amino-ethyl)-piperidine-1-carboxylic acid tert-butyl ester resin:

¹H NMR (400 MHz, CDCl₃): δ 4.05 (br s, 2H), 2.6 (br m, 3H), 2.25 (m, 1H), 1.6 (dd, 2H), 1.4 (s, 9H), 1.2 (m, 2H), 1.1 (m, 2H), 1.0 (d, 3H).

Step 5:

(R,S) 4-[1-(Pyridin-4-ylamino)-ethyl]-piperidine-1-carboxylic acid tert-butyl ester

A mixture of 3 g of 4-(1-amino-ethyl)-piperidine-1-carboxylic acid tert-butyl ester, 2.5 g of 4-bromopyridine hydrochloride, 3.6 g of sodium tert-butoxide, 0.14 g of palladium acetate, 0.38 g of racemic BINAP and 50 mL of THF was heated to reflux for 12 h. The mixture was cooled, diluted with 50 mL of water and concentrated under reduced pressure. The resulting residue was partitioned between 500 mL of chloroform and 200 mL of water. The extracts were dried over magnesium sulfate and concentrated under reduced pressure. Purification by chromatography, eluting with 90:10 chloroform saturated with ammonia: methanol gave 3.5 g of a (R,S) 4-[1-(Pyridin-4-ylamino)-ethyl]-piperidine-1-carboxylic acid tert-butyl ester resin:

¹H NMR (400 MHz, CDCl₃): δ 8.15 (d, 2H), 6.4 (d, 2H), 4.3 (d, 1H), 4.15 (br s, 2H), 3.2 (m, 1H), 2.65 (m, 2H), 2.5 (m, 1H), 1.7 (dd, 2H), 1.6 (m, 1H), 1.42 (s, 9H), 1.25 (m, 2H), 1.15 (m, 2H), 1.1 (d, 3H).

Step 6:

(R,S) 4-[1-(Pyridin-4-ylamino)-ethyl]-piperidine-1-carboxylic acid benzyl ester

A mixture of 0.1 g of 4-[1-(pyridin-4-ylamino)-ethyl]-piperidine-1-carboxylic acid tert-butyl ester and 10 mL of 4N HCl in dioxane was stirred at room temperature for 2 h, then concentrated to dryness. The residue was diluted with 50 mL of chloroform and 1 mL of saturated sodium carbonate, cooled to 0° C. and treated with 0.05 mL of benzyl chloroformate. The resulting solution was allowed to stir for 3 h then separated. The organic layer was dried over magnesium sulfate and concentrated under reduced pressure. Purification by preparative chromatography eluting with 90:10 chloroform saturated with ammonia: methanol gave 0.15 g of a (R,S) 4-[1-(Pyridin-4-ylamino)-ethyl]-piperidine-1-carboxylic acid benzyl ester resin:

¹H NMR (400 MHz, CDCl₃) δ 8.15 (d, 2H), 7.3 (m, 5H), 6.4 (d, 2H), 4.38 (d, 1H), 4.15 (br s, 2H), 3.4 (m, 1H), 2.9 (m, 1H), 2.75 (m, 2H), 1.65 (dd, 2H), 1.6 (m, 1H), 1.32 (m, 4H), 1.1 (d, 3H)

MS (m+1)=340.

The following EXAMPLES A81-A103 were prepared from a primary amine described herein and a chloro-substituted heterocycle using conditions and procedures similar to those described in EXAMPLE A77, Step 6 unless otherwise stated:

### Example A81

N2-[1-(2-Phenyl-ethanesulfonyl)-piperidin-4-ylmethyl]-quinazoline-2,4-diamine

EXAMPLE A81 was prepared from C-[1-(2-phenyl-ethanesulfonyl)-piperidin-4-yl]-methylamine and 2-chloro-quinazolin-4-ylamine (2-chloro-quinazolin-4-ylamine was prepared from 2,4-dichloroquinazoline and ammonia in THF at room temperature; N. B. Chapman, G. M. Gibson, F. G. Mann, J. Chem. Soc., 1947, 890-899):

MS (m+1)=426.

### Example A82

[1-(2-Phenyl-ethanesulfonyl)-piperidin-4-ylmethyl]-(9H-purin-2-yl)-amine

EXAMPLE A82 was prepared from C-[1-(2-phenyl-ethanesulfonyl)-piperidin-4-yl]-methylamine and 2-chloro-9H-purine (2-chloro-9H-purine was prepared according to S. R. Brashears, S. S. Wang, S. G. Bechtolt, B. E. Christensen, J. Am. Chem. Soc., 81:3789-3792(1959):

MS (m+1)=401.

### Example A83

2-{[1-(2-Phenyl-ethanesulfonyl)-piperidin-4-ylmethyl]-amino}-pyrimidine-4-carboxylic acid amide

EXAMPLE A83 was prepared from C-[1-(2-phenyl-ethanesulfonyl)-piperidin-4-yl]-methylamine and 2-chloro-pyrimidine-4-carboxylic acid amide (2-chloro-pyrimidine-4-carboxylic acid amide was prepared according to G. D. Davies, D. E. O'Brien, L. R. Lewis, C. C. Cheng, J. Heterocyclic Chem., 1:130-131(1964):

MS (m+1)=404.

### Example A84

(9-Methyl-9H-purin-6-yl)-[1-(2-phenyl-ethanesulfonyl)-piperidin-4-ylmethyl]-amine

EXAMPLE A84 was prepared from C-[1-(2-phenyl-ethanesulfonyl)-piperidin-4-yl]-methylamine and 6-chloro-9-methyl-9H-purine (6-chloro-9-methyl-9H-purine prepared according to G. B. Eilon, J. Org. Chem., 27:2478-2491(1962):

MS (m+1)=415.

### Example A85

(7-Methyl-7H-purin-6-yl)-[1-(2-phenyl-ethanesulfonyl)-piperidin-4-ylmethyl]-amine

EXAMPLE A85 was prepared from C-[1-(2-phenyl-ethanesulfonyl)-piperidin-4-yl]-methylamine and 6-chloro-7-methyl-7H-purine (6-chloro-7-methyl-7H-purine was prepared according to G. B. Eilon, J. Org. Chem., 27:2478-2491(1962):

MS (m+1)=415.

### Example A86

4-(Pteridin-4-ylaminomethyl)-piperidine-1-carboxylic acid benzyl ester

EXAMPLE A86 was prepared from 4-aminomethyl-piperidine-1-carboxylic acid benzyl ester and 4-methylthio-pteridine (4-methylthio-pteridine was prepared according to A. A. Brown, D. J. Brown,h. C. S. Wood, J. Chem. Soc., 1954, 3832-3839):

MS (m+1)=379.

### Example A87

4-[(7H-Pyrrolo [2,3-d]pyrimidin-4-ylamino)-methyl]-piperidine-1-carboxylic acid benzyl ester

EXAMPLE A87 was prepared from 4-aminomethyl-piperidine-1-carboxylic acid benzyl ester and 4-chloro-7H-pyrrolo[2,3-d]pyrimidine (4-chloro-7H-pyrrolo[2,3-d]pyrimidine was prepared according to U. Lupke, F. Seela, Chem. Ber., 112:3832-3839(1979):

MS (m+1)=366.

### Example A88

4-[(1H-Imidazo[4,5-c]pyridin-4-ylamino)-methyl]-piperidine-1-carboxylic acid benzyl ester

EXAMPLE A88 was prepared from 4-aminomethyl-piperidine-1-carboxylic acid benzyl ester and 7-chloro-3H-imidazo[4,5-b]pyridine (7-chloro-3H-imidazo[4,5-b]pyridine was prepared according to Y. Mizuno, T. Itoh, K Saito, Chem. Pharm. Bull., 12:866-872(1964):

MS (m+1)=366.

### Example A89

(3-Chloro-pyrazin-2-yl)-[1-(2-phenyl-ethanesulfonyl)-piperidin-4-ylmethyl]-amine

EXAMPLE A89 was prepared from C-[1-(2-phenyl-ethanesulfonyl)-piperidin-4-yl]-methylamine and 2,3-dichloropyrazine (refluxing 2-butanol):

MS (m+1)=396.

### Example A90

[1-(2-Phenyl-ethanesulfonyl)-piperidin-4-ylmethyl]-pyrazin-2-yl-amine

EXAMPLE A90 was prepared from (3-chloro-pyrazin-2-yl)-[1-(2-phenyl-ethanesulfonyl)-piperidin-4-ylmethyl]-amine by hydrogenation in ethanol-triethylamine over 5% palladium on carbon, 1 atm of hydrogen:

MS (m+1)=361.

### Example A91

(2-Chloro-5-methyl-pyrimidin-4-yl)-[1-(2-phenyl-ethanesulfonyl)-piperidin-4-ylmethyl]-amine

EXAMPLE A91 was prepared from C-[1-(2-phenyl-ethanesulfonyl)-piperidin-4-yl]-methylamine and 2,4-dichloro-5-methyl-pyrimidine:

MS (m+1)=410.

### Example A92

(5-Methyl-pyrimidin-4-yl)-[1-(2-phenyl-ethanesulfonyl)-piperidin-4-ylmethyl]-amine

EXAMPLE A92 was prepared from (2-chloro-5-methyl-pyrimidin-4-yl)-[1-(2-phenyl-ethanesulfonyl)-piperidin-4-ylmethyl]-amine by hydrogenation in ethanol-triethylamine over 5% palladium on carbon, 1 atm of hydrogen:

MS (m+1)=375.5.

### Example A93

[1-(2-Phenyl-ethanesulfonyl)-piperidin-4-ylmethyl]-pyrimidin-4-yl-amine

EXAMPLE A93 was prepared from C-[1-(2-phenyl-ethanesulfonyl)-piperidin-4-yl]-methylamine and 2,4-dichloro-pyrimidine followed by hydrogenation in ethanol-triethylamine over 5% palladium on carbon, 1 atm of hydrogen:

MS (m+1)=361.5.

### Example A94

(4-Methyl-pyrimidin-2-yl)-[1-(2-phenyl-ethanesulfonyl)-piperidin-4-ylmethyl]-amine

EXAMPLE A94 was prepared from C-[1-(2-phenyl-ethanesulfonyl)-piperidin-4-yl]-methylamine and 2-chloro-4-methyl-pyrimidine:

MS (m+1)=375.5.

### Example A95

5-Fluoro-N-2-[1-(2-phenyl-ethanesulfonyl)-piperidin-4-ylmethyl]-pyrimidine-2,4-diamine

EXAMPLE A95 was prepared from C-[1-(2-phenyl-ethanesulfonyl)-piperidin-4-yl]-methylamine and 2-chloro-5-fluoro-pyrimidin-4-ylamine:

MS (m+1)=394.5.

### Example A96

N2-[1-(2-Phenyl-ethanesulfonyl)-piperidin-4-ylmethyl]-pyrimidine-2,4-diamine

EXAMPLE A96 was prepared from C-[1-(2-phenyl-ethanesulfonyl)-piperidin-4-yl]-methylamine and 2-chloro-pyrimidin-4-ylamine (prepared from 2,4-chloro-pyrimidin-4-ylamine by hydrogenation in ethanol over 5% palladium on carbon, 1 atm of hydrogen): MS (m+1)=376.5.

### Example A97

(3-Methyl-pyrazin-2-yl)-[1-(2-phenyl-ethanesulfonyl)-piperidin-4-ylmethyl]-amine

EXAMPLE A97 was prepared from C-[1-(2-phenyl-ethanesulfonyl)-piperidin-4-yl]-methylamine and 3-bromo-pyrazine-2-carboxylic acid methyl ester followed by reduction with lithium tri-sec-butylborohydride at 0° C. in THF:

MS (m+1)=375.5.

### Example A98

{1-[2-(2-Fluoro-phenyl)-ethanesulfonyl]-piperidin-4-ylmethyl}-pyrimidin-2-yl-amine

EXAMPLE A98 was prepared from 2-(2-fluoro-phenyl)-ethanol as described in EXAMPLE A78, Steps 1-7 above:

MS (m+1)=378.5.

### Example A99

{1-[2-(4-Chloro-phenyl)-ethanesulfonyl]-piperidin-4-ylmethyl}-pyrimidin-2-yl-amine

EXAMPLE A99 was prepared from 2-(4-chloro-phenyl)-ethanol as described in EXAMPLE A78, Steps 1-7 above:

MS (m+1)=396.

### Example A100

Pyrimidin-2-yl-[1-(2-p-tolyl-ethanesulfonyl)-piperidin-4-ylmethyl]-amine

EXAMPLE A100 was prepared from 2-(4-methyl-phenyl)-ethanol as described in EXAMPLE 78, Steps 1-7 above:

MS (m+1)=375.5.

### Example A101

3-(Pteridin-4-ylaminomethyl)-pyrrolidine-1-carboxylic acid benzyl ester

EXAMPLE A101 was prepared from 3-aminomethyl-pyrrolidine-1-carboxylic acid benzyl ester (EXAMPLE A79, Step 3) and 4-methylthio-pteridine (A. A. Brown, D. J. Brown,h. C. S. Wood, J. Chem. Soc., 1954, 3832-3839):

MS (m+1)=365.4.

### Example A102

3-[(9H-Purin-6-ylamino)-methyl]-pyrrolidine-1-carboxylic acid benzyl ester

EXAMPLE A102 was prepared from 3-aminomethyl-pyrrolidine-1-carboxylic acid benzyl ester (EXAMPLE A79, Step 3) and 6-chloro-9H-purine:

MS (m+1)=353.4.

### Example A103

3-Nitro-N⁶-[1-(2-phenyl-ethanesulfonyl)-piperidin-4-ylmethyl]-pyridine-2,6-diamine

EXAMPLE A103 was prepared from C-[1-(2-phenyl-ethanesulfonyl)-piperidin-4-yl]-methylamine and 6-chloro-3-nitro-pyridin-2-ylamine:

MS (m+1)=420.5.

### Example A104

(1H-Imidazo[4,5-b]pyridin-5-yl)-[1-(2-phenyl-ethanesulfonyl)-piperidin-4-ylmethyl]-amine

EXAMPLE A104 was prepared from 3-nitro-N⁶-[1-(2-phenyl-ethanesulfonyl)-piperidin-4-ylmethyl]-pyridine-2,6-diamine (EXAMPLE A103) (1 mmol scale) by hydrogenation in 15 mL of THF/methanol over 0.5 of Raney-nickel under 1 atm of hydrogen for 1 h, followed by immediate conversion of the crude, air sensitive triaminopyridine into the imidazo[4,5 b]pyridine by heating with 5 mL of 96% formic acid and 2 mL of 37% hydrochloric acid at reflux overnight. The free base was liberated with sodium hydroxide and purified by preparative chromatography, eluting with 90:10 chloroform:methanol:

MS (m+1)=400.5.

### Example A105

4-[(1H-Benzoimidazol-4-ylamino)-methyl]-piperidine-1-carboxylic acid benzyl ester

EXAMPLE A105 was prepared from 1H-benzoimidazol-4-ylamine (The 1H-benzoimidazol-4-ylamine was prepared by heating 1.5 g of 3-nitro-benzene-1,2-diamine in 50 mL of triethyl orthoformate with 10 mg of p-toluenesulfonic acid monohydrate at reflux overnight, concentration to dryness under reduced pressure, hydrolysis with refluxing 3N HCl for 1 h and neutralization with NaOH. Then, cooling and collection yielded the 4-nitro-benzimidazole product by filtration. Catalytic reduction using Raney-nickel in ethanol under 1 atm of hydrogen for 1 h gave 1H-benzoimidazol-4-ylamine as an air sensitive solid) and 4-formyl-piperidine-1-carboxylic acid benzyl ester (prepared from 4-(N-methoxy-N-methyl-carbamoyl)-piperidine-1-carboxylic acid benzyl ester, using the procedures described by S. Nahm and S. W. Weinreb, Tetrahedron Letters, 22:3815-3818(1981)) on a 1 mmol scale by reductive amination in 5 mL of 1,2-dichloromethane using sodium triacetoxyborohydride over 0.5 of Raney-nickel under 1 atm of hydrogen for 1 h, followed by immediate conversion of the crude, air sensitive triaminopyridine into the imidazo[4,5b]pyridine by heating with 5 mL of 96% formic acid and 2 mL of 37% hydrochloric acid at reflux overnight. The free base was liberated with sodium hydroxide and purified by preparative chromatography, eluting with 90:10 chloroform:methanol:

MS (m+1)=365.5.

### Example A106

4-[(3-Hydroxy-pyridin-4-ylamino)-methyl]-piperidine-1-carboxylic acid benzyl ester

EXAMPLE A106 was prepared from 4-(3-hydroxy-pyridin-4-ylcarbamoyl)-piperidine-1-carboxylic acid benzyl ester (which was prepared by EDC coupling of 4-amino-pyridin-3-ol and N-benzyloxycarbonyl piperidine-4-carboxylic acid) by borane-THF reduction overnight at room temperature. The reaction was quenched by slow addition of 1N HCl until pH=2, then basified to pH=10 with 10 N NaOH. Extraction with chloroform yielded a crude product which was purified by preparative chromatography, eluting with 90:10 chloroform saturated with ammonia: methanol to give 4-[(3-Hydroxy-pyridin-4-ylamino)-methyl]-piperidine-1-carboxylic acid benzyl ester:

MS (m+1)=342.4.

### Example A107

Step 1:

(8-Benzyl-8-azabicyclo[3.2.1] oct-3-exo-yl)methylamine

In a three-neck flask equipped with an addition funnel, a nitrogen inlet, and a rubber septum was placed a 1M solution of lithium aluminum hydride in tetrahydrofuran (5.5 mL, 5.5 mmol). To that solution, a solution of 8-benzyl-8-azabicyclo[3.2.1]octane-3-exo-carbonitrile (EP 31219 A1 19810701) (1.13 g, 5.0 mmol) in dry tetrahydrofuran was added dropwise via syringe. The resulting mixture was stirred 3 hours at 60° C. The mixture was cooled in an ice-bath and 3N sodium hydroxide solution (25 mL) was added dropwise. The mixture was extracted with ethyl acetate (2×100 mL). The combined extract was washed with water (50 mL) and brine (50 mL), dried (sodium sulfate), filtered, and the solvent was evaporated under reduced pressure to give crude (8-Benzyl-8-azabicyclo[3.2.1]oct-3-exo-yl)methylamine product (0.97 g) as an oil.

¹H NMR (CDCl₃) δ 7.38 (2H, d, J=7 Hz), 7.34-7.23 (3H, m), 3.54 (2H, s), 3.21 (2H, m), 2.55 (2H, d, J=6.5 Hz), 2.01 (2H, m), 1.67 (1H, m), 1.60 (2H, d, J=8 Hz), 1.56-1.34 (6H, m).

Mass spec.: 231.50 (M+1).

Step 2:

(8-Benzyl-8-aza-bicyclo [3.2.1]oct-3-exo-ylmethyl)pyridin-4-yl-amine

To a mixture of (8-benzyl-8-azabicyclo[3.2.1]oct-3-exo-yl)methylamine (0.999 g, 4.3 mmol), 4-bromopyridine hydrochloride (0.719 g, 3.7 mmol), palladium acetate (0.033 g, 0.15 mmol), and (±)-BINAP (0.092 g, 0.15 mmol) in tetrahydrofuran (34 mL) under nitrogen, was added sodium t-butoxide (0.86 g, 8.9 mmol). The mixture was stirred at 70° C. under nitrogen for 18 h. The mixture was diluted with ether (35 mL), washed with brine (2×35 mL), dried (sodium sulfate), filtered, and the solvent was evaporated under reduced pressure to give crude product (1.42 g) as a brown gum. The crude product was flash chromatographed on silica gel, eluting first with methanol:methylene chloride (10:90) to remove impurities, then with methanol:methylene chloride: ammonium hydroxide (10:90:1 increasing to 20:80:2) to give a yellow foam (1.08 g). The foam was triturated with ether to give a crystalline solid. The solid was filtered off and dried in vacuo to give the (8-Benzyl-8-aza-bicyclo[3.2.1]oct-3-exo-ylmethyl)pyridin-4-yl-amine product (0.89 g) as a yellow solid.

¹H NMR (CDCl₃) δ 8.16 (2H, m), 7.39 (2H, d, J=1.5 Hz), 7.32 (2H, m), 7.26 (1H, m), 6.41 (2H, m), 4.25 (1H, br s), 3.55 (2H, s), 3.25 (2H, m), 3.02 (2H, t, J=6 Hz), 2.05 (2H, m), 1.97 (1H, m), 1.55 (6H, m).

Mass spec.: 308.36 (M+1).

Step 3:

(8-Benzyl-8-aza-bicyclo [3.2.1]oct-3-exo-ylmethyl)pyridin-4-yl-carbamic acid tert-butyl ester

A mixture of (8-benzyl-8-aza-bicyclo[3.2.1]oct-3-exo-ylmethyl)pyridin-4-yl-amine (0.707 g, 2.3 mmol), 4-dimethylaminopyridine (0.037 g, 0.30 mmol, 0.13 equiv.), and di-tert-butyl dicarbonate (0.79 g, 3.6 mmol) in acetonitrile was stirred under nitrogen at ambient temperature for 18 h. The mixture was concentrated under reduced pressure and the residue was taken up in methylene chloride (60 mL). The mixture was washed with saturated sodium bicarbonate solution (30 mL), water (30 mL), and brine (30 mL), dried (sodium sulfate), filtered, and the solvent was evaporated under reduced pressure to give a crude product (0.96 g) as an orange gum. The crude product was flash chromatographed on silica gel eluting first with methanol:methylene chloride (10:90), then with methanol:methylene chloride: ammonium hydroxide (10:90:1) to give the (8-Benzyl-8-aza-bicyclo[3.2.1]oct-3-exo-ylmethyl)pyridin-4-yl-carbamic acid tert-butyl ester product (0.930 g) as a yellow oil.

¹H NMR (CDCl₃) δ 8.52 (2H, m), 7.40-7.23 (5H, m), 7.19 (2H, m), 3.60 (2H, d, J=7 Hz), 3.51 (2H, m), 3.18 (2H, br s), 1.99 (3H, m), 1.48 (9H, s), 1.42 (6H, m).

Step 4:

(8-Aza-bicyclo [3.2.1] oct-3-exo-ylmethyl)pyridin-4-yl-carbamic acid tert-butyl ester

A mixture of (8-benzyl-8-aza-bicyclo [3.2.1]oct-3-exo-ylmethyl)pyridin-4-yl-carbamic acid tert-butyl ester (0.917 g, 2.25 mmol) and 10% palladium on carbon (0.60 g) in methanol (25 mL) was hydrogenated (53 psi hydrogen) for 18 h. The catalyst was removed by filtration through Celite. The filter cake was washed with methanol (3×25 mL) and the filtrate was concentrated under reduced pressure to give crude product (0.592 g) as a gum. The crude product was flash chromatographed on silica gel eluting with methanol:methylene chloride: ammonium hydroxide (10:90:1 increasing to 20:80:2) to give product (0.424 g) as a solid white foam.

¹H NMR (CDCl₃) δ 8.53 (2H, m), 7.19 (2H, m), 3.80 (2H, s), 3.64 (2H, d, J=7 Hz), 2.6-2.0 (1H, br s), 2.10 (1H, m), 2.07 (2H, m), 1.63 (6H, m), 1.48 (9H, s).

Step 5:

3-exo-[(tert-Butoxycarbonyl-pyridin-4-yl-amino)methyl]-8-aza-bicyclo[3.2.1]octane-8-carboxylic acid benzyl ester

To a rapidly stirred mixture of (8-aza-bicyclo[3.2.1]oct-3-exo-ylmethyl)pyridin-4-yl-carbamic acid tert-butyl ester (95 mg, 0.30 mmol), sodium bicarbonate (76 mg, 0.90 mmol), methylene chloride (0.8 mL), and water (0.8 mL) cooled in an ice-bath, was added benzyl chloroformate (57 μL, 68 mg, 0.40 mmol). The mixture was stirred 18 h while warming from ice-bath to ambient temperature. The mixture was diluted with dichloromethane (5 mL) and the layers were separated. The organic layer was washed with water (2 mL), and brine (2 mL), dried (sodium sulfate), filtered, and the solvent was evaporated under reduced pressure to give a crude product (112 mg) as a pale yellow oil. The crude product was chromatographed on a 2 mm silica gel prep plate eluting with ethyl acetate: hexane (3:2) to give 3-exo-[(tert-Butoxycarbonyl-pyridin-4-yl-amino)methyl]-8-aza-bicyclo[3.2.1]octane-8-carboxylic acid benzyl ester product (58 mg) as a colorless gum.

¹H NMR (CDCl₃) δ 8.53 (2H, d, J=6 Hz), 7.34 (5H, m), 7.17 (2H, d, J=6 Hz), 5.12 (2H, s), 4.29 (2H, br s), 3.56 (2H, d, J=7 Hz), 2.17 (1H, m), 1.92 (2H, m), 1.55-1.31 (15H, m).

Step 6:

3-exo-(Pyridin-4-ylaminomethyl)-8-aza-bicyclo [3.2.1]octane-8-carboxylic acid benzyl ester hydrochloride

Into a solution of 3-exo-[(tert-butoxycarbonyl-pyridin-4-yl-amino)methyl]-8-aza-bicyclo [3.2.1]octane-8-carboxylic acid benzyl ester (54 mg, 0.12 mmol) in ethyl acetate (1 mL), cooled in an ice-bath, was bubbled hydrogen chloride for 2 minutes. The solution was stirred one hour with ice-bath cooling, de-gassed with nitrogen, then concentrated under reduced pressure. The residual gum was dissolved in methylene chloride (0.5 mL) and the solution was diluted with ether (5 mL) to deposit a gum. The supernatant was decanted, the gum was triturated with ether, and the resulting solid was filtered off and dried in vacuo to give 3-exo-(Pyridin-4-ylaminomethyl)-8-aza-bicyclo[3.2.1]octane-8-carboxylic acid benzyl ester hydrochloride (43 mg) as an off-white solid.

¹H NMR (DMSO-d₆) δ 13.34 (1H, br s), 8.68 (1H, m), 8.19 (1H, br s), 8.06 (1H, br s), 7.36 (5H, m), 6.90 (2H, d, J=7 Hz), 5.08 (2H, s), 4.20 (2H, br s),3.11 (2H, t, J=6 Hz), 2.17 (1H, m), 1.88 (2H, m), 1.65 (4H, m), 1.31 (2H, m).

Mass spec.: 352.41 (M+1).

### Example A108

Step 1:

(8-Benzyl-8-aza-bicyclo [3.2.1]oct-3-exo-ylmethyl)carbamic acid tert-butyl ester

To a solution of (8-benzyl-8-azabicyclo[3.2.1]oct-3-exo-yl)methylamine (EXAMPLE A106, Step 1) (0.65 g, 2.8 mmol) in dichloromethane (30 mL) was added di-tert-butyl dicarbonate (0.65 mL, 0.69 g, 3.0 mmol). The solution was stirred 18 h under nitrogen. The solution was diluted with dichloromethane (50 mL), washed with saturated sodium bicarbonate solution (25 mL), water (25 mL), and brine (25 mL), dried (sodium sulfate), filtered, and the solvent was evaporated under reduced pressure to give a crude product (0.993 g) as a pale yellow solid. A solution of the crude product in ethyl acetate (5 mL) was filtered through a pad of silica gel, eluting with ethyl acetate:hexane (2:1). The filtrate was evaporated under reduced pressure to give (8-benzyl-8-aza-bicyclo[3.2.1]oct-3-exo-ylmethyl)carbamic acid tert-butyl ester product (0.898 g) as a white solid.

¹H NMR (CDCl₃) δ 7.37 (2H, d, J=7 Hz), 7.30 (2H, t, J=7 Hz), 7.24 (1H, m), 4.55 (1H, br s), 3.53 (2H, s), 3.19 (2H, s), 2.99 (2H, m), 2.00 (2H, m), 1.80 (1H, m), 1.55 (4H, m), 1.44 (11H, m).

Step 2:

(8-Aza-bicyclo[3.2.1]oct-3-exo-ylmethyl)carbamic acid tert-butyl ester

A mixture of tert-butyl (8-benzyl-8-azabicyclo[3.2.1]oct-3-exo-yl)methylcarbamate (0.892 g, 2.7 mmol) and 10% palladium on carbon (0.55 g) in methanol (50 mL) was hydrogenated under a hydrogen balloon for 18 h. The catalyst was removed by filtration through Celite. The filter cake was washed with methanol (3×25 mL) and the filtrate was concentrated under reduced pressure to give crude (8-aza-bicyclo[3.2.1]oct-3-exo-ylmethyl)carbamic acid tert-butyl ester product (0.607 g) as a white solid.

¹H NMR (CDCl₃) δ 4.57 (1H, br s), 3.53 (2H, s), 2.96 (2H, m), 1.95-1.77 (4H, m), 1.72-1.50 (4H, m), 1.44 (9H, m), 1.24 (2H, m).

Mass spec.: 241.32 (M+1).

Step 3:

3-exo-(tert-Butoxycarbonylamino-methyl)-8-aza-bicyclo[3.2.1]octane-8-carboxylic acid benzyl ester

To a mixture of tert-butyl 8-azabicyclo[3.2.1]oct-3-exo-ylmethylcarbamate (0.84 g, 3.5 mmol) in acetonitrile (35 mL) was added 1-{[(benzyloxy)carbonyl]oxy}pyrrolidine-2,5-dione (0.87 g, 3.5 mmol). The mixture was stirred 18 h under nitrogen. The resulting solution was concentrated under reduced pressure. The residue was partitioned between ethyl acetate (150 mL) and water (75 mL) and the layers were separated. The organic layer was washed with water (2×75 mL) and brine (50 mL), dried (sodium sulfate), filtered, and the solvent was evaporated under reduced pressure to give a crude product (1.31 g) as a white solid. The crude product was purified by flash column chromatography on silica gel, eluting with ethyl acetate:hexane (30:70 increasing to 50:50) to give the 3-exo-(tert-butoxycarbonylamino-methyl)-8-aza-bicyclo [3.2.1]octane-8-carboxylic acid benzyl ester product (0.95 g) as a white solid.

¹H NMR (CDCl₃) δ 7.36 (5H, m), 5.13 (2H, s), 4.56 (1H, br s), 4.32 (2H, br s), 2.94 (2H, m), 2.00 (3H, m), 1.62 (4H, m), 1.48-1.25 (11H, m).

Mass spec.: 375.39 (M+1).

Step 4:

3-exo-Aminomethyl-8-aza-bicyclo [3.2.1]octane-8-carboxylic acid benzyl ester

Benzyl 3-exo-{[(tert-butoxycarbonyl)amino]methyl}-8-azabicyclo[3.2.1]octane-8-carboxylate (0.94 g, 2.5 mmol) was placed in a round-bottom flask under nitrogen and cooled in an ice-bath. Trifluoroacetic acid (6 mL) was added dropwise and the mixture was stirred one hour with ice-bath cooling. The mixture was poured into ice-cold 5N sodium hydroxide solution (16 mL) and the aqueous mixture was extracted with methylene chloride (4×50 mL). The extract was washed with brine (50 mL), dried (sodium sulfate), filtered, and the solvent was evaporated under reduced pressure to give product (0.59 g, 86%) as a colorless oil.

¹H NMR (CDCl₃) δ 7.36 (5H, m), 5.14 (2H, s), 4.33 (2H, br s), 2.52 (2H, d, J=6 Hz), 1.96 (2H, m), 1.88 (1H, m), 1.67 (2H, d, J=7 Hz), 1.61 (2H, m), 1.42-1.25 (4H, m).

Mass spec.: 275.34 (M+1).

Step 5:

3-exo-[(9H-Purin-6-ylamino)-methyl]-8-aza-bicyclo[3.2.1]octane-8-carboxylic acid benzyl ester

A solution of 3-exo-aminomethyl-8-aza-bicyclo[3.2.1]octane-8-carboxylic acid benzyl ester (27 mg, 0.10 mmol), 6-chloropurine (31 mg, 0.20 mmol), and diisopropylethylamine (35 μL, 0.20 mmol) in isopropanol (2 mL) was heated at reflux for 18 h. The resulting mixture was concentrated under reduced pressure and the residue was taken up in ethyl acetate (3 mL). The resulting mixture was washed with saturated sodium bicarbonate solution (1 mL), water (2×1 mL), and brine (1 mL), dried (sodium sulfate), filtered, and the solvent was evaporated under reduced pressure to give a crude product (39 mg) as a yellow solid. The solid was triturated in hot ethyl acetate (1 mL), the mixture cooled to ambient temperature, and the solid precipitate filtered off and dried in vacuo to give the 3-exo-[(9H-purin-6-ylamino)-methyl]-8-aza-bicyclo [3.2.1]octane-8-carboxylic acid benzyl ester product (28 mg) as a white solid.

¹H NMR (DMSO-d₆) δ 12.86 (1H, br s), 8.16 (1H, s), 8.07 (1H, s), 7.61 (1H, br s), 7.35 (5H, m), 5.08 (2H, d, J=2 Hz), 4.17 (2H, br s), 3.32 (2H, m), 2.26 (1H, m), 1.86 (2H, br s), 1.61 (4H, m), 1.34 (2H, m).

Mass spec.: 393.36 (M+1).

### Example A109

3-exo-[(3-Chloropyrazin-2-ylamino)methyl]-8-aza-bicyclo[3.2.1]octane-8-carboxylic acid benzyl ester

Employing the procedure substantially as described for 3-exo-[(9H-purin-6-ylamino)-methyl]-8-aza-bicyclo[3.2.1]octane-8-carboxylic acid benzyl ester (EXAMPLE A108), but substituting 2,3-dichloropyrazine for 6-chloropurine, the crude product (51 mg) was obtained as an oil. The crude product was filtered through a pad of silica gel eluting with ethyl acetate:hexane (2:1), and the filtrate was concentrated under reduced pressure. The residual oil was dissolved in ether, the solvent evaporated under reduced pressure, and the residue dried in vacuo to give 3-exo-[(3-chloropyrazin-2-ylamino)methyl]-8-aza-bicyclo [3.2.1]octane-8-carboxylic acid benzyl ester (24 mg) as a yellow gum.

¹H NMR (CDCl₃) δ 7.93 (1H, d, J=3 Hz), 7.56 (1H, d, J=3 Hz), 7.36 (5H, m), 5.20 (1H, m), 5.15 (2H, s), 4.34 (2H, br s), 3.32 (2H, m), 2.21 (1H, m), 1.97 (2H, m), 1.66 (4H, m), 1.60-1.40 (2H, m).

Mass spec.: 387.27 (M+1).

### Example A110

[8-(2-Phenylethanesulfonyl)-8-aza-bicyclo[3.2.1]oct-3-exo-ylmethyl]pyrimidin-2-yl-amine

Step 1:

[8-(2-trans-Phenylethenesulfonyl)-8-aza-bicyclo[3.2.1]oct-3-exo-ylmethyl]carbamic acid tert-butyl ester

To a solution of tert-butyl 8-azabicyclo[3.2.1]oct-3-exo-ylmethylcarbamate (EXAMPLE A106, Step 1) (0.60 g, 2.5 mmol) and diisopropylethylamine (0.52 mL, 0.39 g, 3.0 mmol) in methylene chloride (15 mL), under nitrogen cooled in an ice-bath, was added dropwise over 10 minutes a solution of trans-2-phenylethenesulfonyl chloride (0.57 g, 2.8 mmol) in methylene chloride (10 mL). The resulting mixture was stirred 18 h under nitrogen while warming from ice-bath to ambient temperature. The solution was diluted with dichloromethane (125 mL), washed with 1N sodium hydroxide solution (50 mL), water (50 mL), and brine (50 mL), dried (sodium sulfate), filtered, and the solvent was evaporated under reduced pressure to give a crude product (0.95 g) as yellow gum. The crude product was purified by flash column chromatography on silica gel, eluting with ethyl acetate:hexane (33:67 increasing to 50:50) to give the [8-(2-trans-phenylethenesulfonyl)-8-aza-bicyclo[3.2.1]oct-3-exo-ylmethyl]carbamic acid tert-butyl ester product (0.63 g) as a colorless gum.

¹H NMR (CDCl₃) δ 7.50-7.40 (6H, m), 6.65 (1H, d, J=15 Hz), 4.58 (1H, br s), 4.24 (2H, br s), 3.00 (2H, m), 1.96 (3H, m), 1.69 (3H, m), 1.54 (3H, m), 1.44 (9H, m).

Step 2:

[8-(2-Phenylethanesulfonyl)-8-aza-bicyclo [3.2.1]oct-3-exo-ylmethyl]carbamic acid tert-butyl ester

A mixture of [8-(2-trans-phenylethenesulfonyl)-8-aza-bicyclo[3.2.1]oct-3-exo-ylmethyl]carbamic acid tert-butyl ester (0.61 g, 1.5 mmol) and 20% palladium hydroxide on carbon (0.30 g) in ethanol (50 mL) was hydrogenated (52 psi hydrogen) for 18 h. The catalyst was removed by filtration through Celite. The filter cake was washed with ethanol (3×25 mL) and the filtrate was concentrated under reduced pressure to give crude [8-(2-phenylethanesulfonyl)-8-aza-bicyclo[3.2.1]oct-3-exo-ylmethyl]carbamic acid tert-butyl ester product (0.64 g) as a gum.

¹H NMR (CDCl₃) δ 7.35-7.20 (5H, m), 4.56 (1H, br s), 4.24 (2H, br s), 3.24 (2H, m), 3.11 (2H, m), 2.98 (2H, t, J=6 Hz), 2.02 (2H, m), 1.92 (1H, m), 1.74-1.51 (4H, m), 1.44 (9H, s), 1.37 (2H, m).

Step 3:

C-[8-(2-Phenylethanesulfonyl)-8-aza-bicyclo[3.2.1]oct-3-exo-yl]methylamine

A solution of crude [8-(2-phenylethanesulfonyl)-8-aza-bicyclo[3.2.1]oct-3-exo-ylmethyl]carbamic acid tert-butyl ester (0.64 g, 1.5 mmol) in dioxane (2 mL) and 3N hydrochloric acid (2 mL) was heated at reflux for 3 h. The solvent was removed under reduced pressure. The aqueous residue was cooled in an ice-bath and made basic with 3N sodium hydroxide solution. The aqueous mixture was extracted with methylene chloride (4×20 mL). The organic layer was washed with brine (20 mL), dried (sodium sulfate), filtered, and the solvent was evaporated under reduced pressure to give a crude product (0.404 g) as a pale yellow oil. A solution of the crude product in methylene chloride was filtered through a pad of silica gel eluting with methanol:methylene chloride: ammonium hydroxide (20:80:2) to give the C-[8-(2-phenylethanesulfonyl)-8-aza-bicyclo[3.2.1]oct-3-exo-yl]methylamine product (0.324 g) as a yellow oil.

¹H NMR (CDCl₃) δ 7.32 (2H, m), 7.26 (1H, m), 7.21 (2H, d, J=7 Hz), 4.24 (2H, m), 3.24 (2H, m), 3.11 (2H, m), 2.56 (2H, d, J=6 Hz), 2.03 (2H, m), 1.82-1.65 (5H, m), 1.37 (4H, m).

Mass spec.: 309.33 (M+1).

Step 4:

[8-(2-Phenylethanesulfonyl)-8-aza-bicyclo[3.2.1]oct-3-exo-ylmethyl]pyrimidin-2-yl-amine

A solution of C-[8-(2-Phenylethanesulfonyl)-8-aza-bicyclo[3.2.1]oct-3-exo-yl]methylamine (31 mg, 0.10 mmol), 2-bromopyrimidine (32 mg, 0.20 mmol), and diisopropylethylamine (35 μL, 0.20 mmol) in isopropanol (2 mL) was heated at reflux for 18 h. The mixture was concentrated under reduced pressure and the residue was taken up in ethyl acetate (3 mL). The resulting mixture was washed with saturated sodium bicarbonate solution (1 mL), water (2×1 mL), and brine (1 mL), dried (sodium sulfate), filtered, and the solvent was evaporated under reduced pressure to give a crude product (39 mg) as a yellow solid. The crude product was chromatographed on a 1 mm silica gel prep plate eluting with ethyl acetate:hexane (2:1) to give a colorless gum (27 mg). The gum was crystallized from ethyl acetate, the precipitate filtered off, and dried in vacuo to give the [8-(2-phenylethanesulfonyl)-8-aza-bicyclo[3.2.1]oct-3-exo-ylmethyl]pyrimidin-2-yl-amine product (23 mg) as a white solid.

¹H NMR (CDCl₃) δ 8.26 (2H, d, J=5 Hz), 7.32 (2H, m), 7.26 (1H, m), 7.21 (2H, d, J=7 Hz), 6.53 (1H, t, J=5 Hz), 5.11 (1H, m), 4.25 (2H, m), 3.31 (2H, t, t, J=6.5 Hz), 3.24 (2H, m), 3.12 (2H, m), 2.03 (3H, m), 1.74 (4H, m), 1.46 (2H, m).

Mass spec.: 387.31 (M+1).

### Example A111

1-[4-(Pyrimidin-2-ylaminomethyl)-piperidin-1-yl]-4-thiophen-2-yl-butan-1-one

Piperidin-4-ylmethyl-pyrimidin-2-yl-amine (EXAMPLE A16) was hydrogenated as described in EXAMPLE A30, Step 1. The resulting piperidine was combined with EDC (1.3equiv.), HOBT (1.0equiv.), and 4-thiophen-2-yl-butyric acid (1.0equiv.) in DMF and stirred for 2 h. The resulting reaction solution was partitioned into ethylacetate and aqueous sodium bicarbonate. The organic layer was seperated and washed with pH 4.5 citric acid buffer (10% citric acid and sodium hydroxide), dried (sodium sulfate), and concentrated to yield the desired 1-[4-(pyrimidin-2-ylaminomethyl)-piperidin-1-yl]-4-thiophen-2-yl-butan-1-one.

M.S. (M+1): 345.25

### Example A112

3-Phenyl-1-[4-(pyrimidin-2-ylaminomethyl)-piperidin-1-yl]-propan-1-one

The title compound was prepared as described in EXAMPLE A111, except substituting 4-thiophen-2-yl-butyric acid with 3-phenyl-propionic acid.

M.S. (M+1): 325.28

### Example A113

(2-Phenyl-cyclopropyl)-[4-(pyrimidin-2-ylaminomethyl)-piperidin-1-yl]-methanone

The title compound was prepared as described in EXAMPLE A111, except substituting 4-thiophen-2-yl-butyric acid with 2-phenyl-cyclopropanecarboxylic acid.

M.S. (M+1): 337.27

### Example A114

2-Phenoxy-1-[4-(pyrimidin-2-ylaminomethyl)-piperidin-1-yl]-ethanone

The title compound was prepared as described in EXAMPLE A111, except substituting 4-thiophen-2-yl-butyric acid with phenoxy-acetic acid.

M.S. (M+1): 341.27

### Example A115

4-(Pyridin-4-ylaminomethyl)-piperidine-1-carboxylic acid thiophen-3-ylmethyl ester

The title compound was prepared as described in EXAMPLE A30, except substituting 3-fluorobenzyl alcohol with thiophen-3-yl-methanol.

M.S. (M+1): 332.31

### Example A116

N-benzyl-N′-cyano-N″-[4-(pyridin-4-ylaminomethyl)piperidinyl]guanidine

To a solution of diphenyl cyanocarbonimidate (0.44 mmol) in THF (3 mL) at −78° C. was added benzyl amine (0.44 mmol, in 2 mL THF) dropwise. The cooling bath was removed, and after reaching 20° C., piperidin-4-ylmethyl-pyridin-4-yl-amine (0.44 mmol, in 2 mL DMF, EXAMPLE A30) was added. The resulting reaction mixture was heated to 90° C. for 14 h, cooled, the volatiles were removed under vacuum, and the resulting residue purified by silica gel chromatography.

M.S. (M+1): 349.38

Intermediates:

Intermediate 1a:

Carbonic acid 2,5-dioxo-pyrrolidin-1-yl ester 4-methyl-benzyl ester

Disuccinimidyl carbonate (5.03 g, 19.65 mmol) in 30 mL MeCN and 30 mL DCM was treated with 4-methylbenzyl alcohol (2.4 g, 19.6 mmol) followed by DMAP (1.20 g, 9.82 mmol). The resulting cloudy reaction mixture was stirred overnight at rt, poured into 100 mL water, and partitioned. The organic layer was dried over anhydrous sodium sulfate and the solvent evaporated. The solid thus obtained was stirred with approx. 25 mL ether, filtered, and the resulting product was washed with a small volume of ether and dried.

Ref: Chem. Pharm. Bull., 38(1):110-115(1990).

The following compounds were similarly prepared in the manner described above for INTERMEDIATE la:

Intermediate 1b:

Carbonic acid 2,5-dioxo-pyrrolidin-1-yl ester 4-chloro-benzyl ester

Intermediate 1c:

Carbonic acid 2,5-dioxo-pyrrolidin-1-yl ester 4-fluoro-benzyl ester

Intermediate 1d:

Carbonic acid 2,5-dioxo-pyrrolidin-1-yl ester 4-ethyl-benzyl ester

Intermediate 1e:

Carbonic acid 2,5-dioxo-pyrrolidin-1-yl ester 4-isopropyl-benzyl ester

Utilizing the carbonic acid derivatives described above as INTERMEDIATES 1a-1e, and following the procedure described below in EXAMPLE 15, step 1, the following INTERMEDIATES 2a-2e were obtained:

Intermediate 2a:

4-Methylbenzyl 4(aminomethyl)piperidine-1-carboxylate

Intermediate 2b:

4-Chlorobenzyl 4-(aminomethyl)piperidine-1-carboxylate

Intermediate 2c: 4-Fluorobenzyl 4-(aminomethyl)piperidine-1-carboxylate

Intermediate 2d:

4-Ethylbenzyl 4-(aminomethyl)piperidine-1-carboxylate

Intermediate 2e:

4-Isopropylbenzyl 4-(aminomethyl)piperidine-1-carboxylate

The carboxylic acids used in the coupling steps were either commercially available or prepared according to the following references:

| Name | Reference |
|---|---|
| 6-Hydroxy-pyridazine-3- | M. Morishita, |
| carboxylic acid | Chem. Pharm. Bull., |
| | 42: 371(1994). |
| 4-Methanesulfonylamino- | L. Exner, Collect Czech |
| benzoic acid | Chem. Comm, 35: 1371- |
| | 1374(1970). |
| 4-Hydroxy-3-iodo-benzoic | L. C. King, et al., J. Amer. |
| acid | Chem. Soc., 67: 2089(1945). |
| 3-Fluoro-4-hydroxy- | J. Minor et al., J. Org. |
| benzoic acid | Chem., (1952), 17, 1425. |
| 2-Fluoro-4-hydroxy- | G. Gray et al., Mol. Cryst. |
| benzoic acid | Liq. Cryst., 67: 1-24 |
| | (1981). |
| Thiazole-4-carboxylic | H. Erlenmeyeir et al., Helv. |
| acid | Chim. Acta., 28: 362(1945). |
| 2H-Pyrazole-3-carboxylic | Sokolov et al., J. Gen. |
| acid | Chem. USSR (Eng.) |
| | 52: 2291(1982). |
| 5-Oxo-4,5-dihydro-1H- | Gehlen Ann (1952) |
| [1,2,4]triazole-3- | 577, 237-241. |
| carboxylic acid | |
| Thiazole-5-carboxylic | H. Erlenmeyer et al., |
| acid | Helv. Chan. Acta., |
| | 30: 1865(1947). |
| 2-Bromo-isonicotinic | A. Campbell et al., |
| acid | Austral. J. Chem., |
| | 24: 377(1971). |
| 5-Methyl-3H-imidazole-4- | G. Wellman et al., |
| carboxylic acid | Synthesis 356(1984). |
| 2-Methyl-1H-pyrrole-3- | E. Benary, Chemische |
| carboxylic acid | Berichte, 44: 493(1911). |
| Oxazole-5-carboxylic | U.S. Pat. No. 4,785,012 |
| acid | |
| 5-Ethyl-2-methyl-2H- | H. A. DeWald et al., |
| pyrazole-3-carboxylic | J. Med. Chem., |
| acid | 16: 1346(1973). |
| 6-Chloro-imidazo [1,2- | WP 96/25414 |
| a]pyridine-2- | |
| carboxylic acid | |
| 4-Bromo-thiophene-3- | Tserng, K. et al., J. Org. |
| carboxylic acid | Chem., 40: 172(1975). |
| 1H-Imidazole-2- | Galeazzi, E. et al., J. Org. |
| carboxylic acid | Chem., 60: 1090(1995). |
| 3-Bromo-isonicotinic | J. Dejardin et al., Bull. Soc. |
| acid | Chim. Fr., 530(1976). |
| [1,6]Naphthyridine- | L. Chan et al., J. Med. |
| 2-carboxylic acid | Chem., 42: 3023(1999). |
| 1-Methyl-1H-imidazole- | Shirley, D. A. et al., |
| 2-carboxylic acid | J. Amer. Chem. Soc., |
| | 79: 4922(1957). |
| Isoxazole-3-carboxylic | R. Cramer et al., J. Org. |
| acid | Chem., 26: 2976(1961). |
| 6-Bromo-nicotinic acid | H. H. Bradlow et al., J. Org. |
| | Chem., 14: 509(1949). |
| 2-Methyl-thiazole-4- | E. Jones et al., J. Chem. |
| carboxylic acid | Soc., 87(1946). |
| Pyrimidine-2-carboxylic | A. Holland, Chem. Ind. |
| acid | (London), 786(1954). |
| 1,4,5,6-Tetrahydro- | N. Auwers, Justus Liebigs |
| cyclopentapyrazole- | Annalen der Chemie, 536: 97-109(1938). |
| 3-carboxylic acid | |
| 5-Methyl-thiazole-4- | G. D. Hartman et al., |
| carboxylic acid | Synthesis, 681(1976). |
| 5-Methyl-2H-[1,2,4]triazole- | J. Dost, Z. Chem., |
| 3-carboxylic acid | 26: 203(1986). |
| 4-Phenyl-thiazole-2- | R. Canas et al., |
| carboxylic acid | Ann. Rev. Soc. Esp. Fis. |
| | Quim., 50: 609-614(1954). |
| 2-Methyl-thiazole-5- | A. Schöberl et al., |
| carboxylic acid | Ber, 73: 1240(1940). |
| 2-Methyl-thiophene-3- | E. Bullock et al., Can. J. |
| carboxylic acid | Chem., 55: 895(1977). |
| Pyrimidine-4-carboxylic | G. A. Archer et al., J. Med. |
| acid | Chem. 16: 1312(1977). |
| 1-Methyl-1H-pyrazole-3- | C. Wijnberger et al., J. |
| carboxylic acid | Heterocycl. Chem., 6: 545(1969). |
| 2-Methyl-2H-pyrazole-3- | C. Wijnberger et al., J. |
| carboxylic acid | Heterocycl. Chem., 6: 545(1969). |
| [1,2,5]Thiadiazole-3- | L. M. Weinstock et al., Adv. |
| carboxylic acid | Heterocycl. Chem., 9: 107(1968). |
| 5-Bromo-pyridine-2- | L. W. Deady et al., Austral. |
| carboxylic acid | J. Chem., 24: 385(1971). |
| Pyrimidine-5-carboxylic | H. Bredereck et al., Liebigs Ann. |
| acid | Chem,.(1972), 766, 73(1972). |
| Pyrazolo[1,5-a]pyrimidine- | Khan et al., J. Heterocycl. |
| 3-carboxylic acid | Chem., 7: 247(1970). |
| Benzothiazole-2-carboxylic | A. Buraway et al., J. Chem. |
| acid | Soc., 648(1956). |
| 3,5-Dimethyl-1H-pyrrole-2- | H. Fischer et al.,Chemische |
| carboxylic acid | Berichte, 56: 1194(1923). |
| 3-Methyl-isonicotinic acid | R. S. Miu et al., Chem. |
| | Abstract., 84: 150463(1976). |
| 2-Methyl-isonicotinic acid | R. Adams et al., J. Amer. |
| | Chem. Soc., 76: 3168(1954). |
| 2-Methoxy-6-methyl- | WO 00/17163 |
| isonicotinic acid | |
| 6-Amino-pyridazine-3- | S. Mitsui, Chem. Abstract., |
| carboxylic acid | 5275(1959). |
| 3-methyl-3H-imidazole- | EP 0306868 |
| 4-carboxylic acid | |

### Example 1

4-[(4-Hydroxy-benzoylamino)-methyl]-piperidine-1-carboxylic acid benzyl ester

Step 1:

Preparation of Benzyl 4-(aminomethyl)piperidine-1-carboxylate

4-Aminomethylpiperidine (40 g, 350 mmol) and benzaldehyde (37.3 mL, 368 mmol) in toluene (600 mL) were heated to reflux under dean stark conditions for 2 h. The resulting reaction mixture was cooled to room temperature and 500 mL dichloromethane was added. The resulting solution was cooled to 5° C. and treated with N-(benzyloxycarbonyloxy)succinimide (91.7 g, 368 mmol). After 10 min, the cooling bath was removed and the reaction mixture stirred for 1 h. The solvents were evaporated and the resulting residue was stirred with 400 mL THF and 400 mL 2M HCl for 1 h. The mixture was concentrated to remove organics and was then extracted with ether (3×300 mL). The aqueous phase was adjusted to pH14 with 50% NaOH and extracted with ethyl acetate. The organic layer was washed with water and brine, dried over anhydrous sodium sulfate, and the solvent evaporated to give benzyl 4-(aminomethyl)piperidine-1-carboxylate as an oil.

¹H NMR 500 MHz (8, CDCl₃) δ: 7.4-7.2 (m, 5H); 5.12 (s, 2H); 4.20 (brs, 2H); 2.77 (brs, 2H); 2.58 (d, J=6.6 Hz, 2H) 1.9-1.7 (m, 2H); 1.0-1.5 (m, 5“I).

Step 2:

Preparation of 4-[(4-Hydroxy-benzoylamino)-methyl]-piperidine-1-carboxylic acid benzyl ester

To a mixture of 4-hydroxybenzoic acid (2.5 g, 0.0182 mol), 1-hydroxybenzotriazole hydrate (3.33 g, 0.0218 mol), benzyl 4-(aminomethyl)piperidine-1-carboxylate (4.5 g, 0.0182 mol) and triethylamine (3.03 mL, 0.0218 mol) in DMF (30 mL) was added 1-ethyl-3-(3-dimethylaminopropyl)carbodiimide hydrochloride (4.2 g, 0.0218 mol) and the mixture allowed to stir at rt for 18 h. The mixture was quenched into water (200 mL) and extracted with ethyl acetate (200 mL). The ethyl acetate extract was washed with 10% aqueous sodium bicarbonate (100 mL), brine (50 mL), dried over sodium sulfate, and filtered. The filtrate was concentrated in vacuo and the residue chromatographed on silica using 10-20% acetone/dichloromethane to give 6.3 g of 4-[(4-hydroxy-benzoylamino)-methyl]-piperidine-1-carboxylic acid benzyl ester as a foam. The foam was dissolved in hot isopropyl acetate (125 mL), filtered, and allowed to cool and crystallize. The reaction volume was reduced in vacuo to 50 mL, allowed to stir overnight at rt and filtered. The resulting solid was dried in vacuo (50° C.) yielding the 4-[(4-hydroxy-benzoylamino)-methyl]-piperidine-1-carboxylic acid benzyl ester.

M. P. 122-123° C. M.S(M+1): 369.

¹H NMR 300 MHz (δ, CDCl₃) δ: 7.64 (d, 2H); 7.4-7.2 (m, 5H); 6.86 (d, 2H); 6.18(m, 1H); 5.85(s, 1H); 5.15 (s, 2H); 4.20 (brs, 2H); 3.35 (brs, 2H); 2.77 (brs, 2H); 1.9-1.7 (m, 311); 1.3-1.1 (m, 2H).

Analysis Calcd. for C₂₁H₂₄N₂O4: C, 68.46; H, 6.57; N, 7.60; Found: C, 68.23; H, 6.61; N, 7.48.

The following compounds were prepared in a manner similar to that used above for the preparation of 4-[(4-hydroxy-benzoylamino)-methyl]-piperidine-1-carboxylic acid benzyl ester, using the appropriate acid in place of the 4-hydroxybenzoic acid. References or experimental procedures are shown for the preparation of non-commercially available acids. Appropriately substituted benzyl 4-(aminomethyl)piperidine-1-carboxylates were prepared in a similar manner to that described above in EXAMPLE 1, step 1, with the necessary N-(benzyloxycarbonyloxy)succinimides prepared as previously described (Chem. Pharm Bull 1990, 38(1) 110-115).

| EX. | Name | Data |
|---|---|---|
| 2. | 4-{[(Pyrazine-2-carbonyl)-amino]-methyl}- | M. S(M+1): 370 ¹H |
| | piperidine-1-carboxylic acid benzyl ester | NMR 300 MHz(δ, |
| | | CDCl₃)δ: 12.10 |
| | | (brs, 1H); 8.02(d, |
| | | 1H, J=2.5 Hz); 7.77 |
| | | (dd, 1H, J=7.7 and |
| | | 2.5 Hz); 7.4-7.2(m, |
| | | 5H); 6.59(d, 2H, |
| | | J=7.7 Hz); 6.12(m, |
| | | 1H); 5.12(s, 2H); |
| | | 4.20(brs, 2H); 3.30 |
| | | (brs, 2H); 2.77(brs, |
| | | 2H); 2.0-1.8(m, |
| | | 3H); 1.3-1.1(m, |
| | | 2H). |
| 3. | 4-{[(3-Amino-pyridine-4-carbonyl)-amino]- | M.S(M+1): 369 |
| | methyl}-piperidine-1-carboxylic acid benzyl ester | NMR(300 MHz, |
| | | CDCl₃)δ: all broad |
| 4. | 4-{[(6-Hydroxy-pyridazine-3-carbonyl)-amino]- | M.S(M+1): 371 |
| | methyl}-piperidine-1-carboxylic acid benzyl ester | NMR(300 MHz, |
| | | CDCl₃)δ: 11.55 |
| | | (brs, 1H); 8.04(d, |
| | | 1H, J=9.8 Hz); 7.4- |
| | | 7.1(m, 5 H); 7.04 |
| | | (d, 1H, 9.8 Hz); |
| | | 5.12(s, 2H); |
| | | 4.22(brs, 2H); 3.30 |
| | | (brs, 2H); 2.80(m, |
| | | 2H); 1.8-1.6(m, |
| | | 3H); 1.3-1.1(m, 2H) |
| 5. | 4-[(4-Methanesulfonylamino-benzoylamino)- | M.S(M+1): |
| | methyl]-piperidine-1-carboxylic acid benzyl | 446NMR(300 MHz, |
| | ester | CDCl₃))δ: 7.75(d, |
| | | 2H, J=8.6 Hz); 7.4- |
| | | 7.2(m, 5H); 7.25(d, |
| | | 2H, J=8.6 Hz); 6.95 |
| | | (brs, 1H); 6.25(brs, |
| | | 1H); 5.12(s, 2H); |
| | | 4.21(brs, 2H); 4.36 |
| | | (brs, 2H); 3.05(s, |
| | | 3H); 2.78(brs, 2H); |
| | | 1.9-1.6(m, 3H); 1.3- |
| | | 1.1(m, 2H). |
| 6. | 4-[(2,4-Dihydroxy-benzoylamino)-methyl]- | M.S(M+1): |
| | piperidine-1-carboxylic acid benzyl ester | 385NMR(300 MHz, |
| | | CDCl₃))δ: 12.55(s, |
| | | 1H);7.5-7.3(m, 5H); |
| | | 7.22(d, 1H, J=8.6 |
| | | Hz); 6.41(d, 1H, |
| | | J=2.5 Hz); 6.34(dd, |
| | | 1H, J=8.6 and 2.5 |
| | | Hz); 6.22(m, 1H); |
| | | 5.13(s, 2H); 4.22 |
| | | (brs, 2H); 3.33(brs, |
| | | 2H); 2.79(brs, 2H); |
| | | 1.8-1.6(m, 3H); 1.3- |
| | | 1.0(m, 2H). |
| 7. | 4-[(3,4-Dihydroxy-benzoylamino)-methyl]- | M.S(M+1): |
| | piperidine-1-carboxylic acid benzyl ester | 385NMR(300 MHz, |
| | | CDCl₃))δ: 7.57(d, |
| | | 1H, J=1.6 Hz); 7.5- |
| | | 7.3(m, 5H); 7.10 |
| | | (dd, 1H, J=8.2 and |
| | | 1.6 Hz); 6.86(d, 1H, |
| | | J=8.2 Hz); 6.30(m, |
| | | 1H); 5.12(s, 2H); |
| | | 4.18(brs, 2H); 3.32 |
| | | (brs, 2H); 2.76(brs, |
| | | 2H); 1.8-1.4(m, |
| | | 3H); 1.3-1.0(m, |
| | | 2H). |
| 8. | 4-[(4-Hydroxy-3-iodo-benzoylamino)-methyl]- | M.S(M+1): 495 |
| | piperidine-1-carboxylic acid benzyl ester | NMR(300 MHz, |
| | | CDCl₃))δ: 8.11(d, |
| | | 1H, J=2.1 Hz); |
| | | 7.63(dd, 1H, J=8.4 |
| | | and 2.1 Hz); 7.5- |
| | | 7.3(m, 5H); 7.00(d, |
| | | 1H, J=8.4 Hz); 6.10 |
| | | (m, 1H); 5.12(s, |
| | | 2H); 4.21(brs, 2H); |
| | | 3.33(brs, 2H); 2.78 |
| | | (brs, 2H); 1.8-1.6 |
| | | (m, 3H); 1.3-1.0(m, |
| | | 2H). |
| 9. | 4-[(3-Fluoro-4-hydroxy-benzoylamino)-methyl]- | M.S(M+1): 387 |
| | piperidine-1-carboxylic acid benzyl ester | NMR(300 MHz, |
| | | CDCl₃))δ: 7.56(dd, |
| | | 1H, J=11.0 and 1.9 |
| | | Hz); 7.5-7.3(m, 6H); |
| | | 7.03(t, 1H, J=8.4 |
| | | Hz); 6.16(m, 1H); |
| | | 5.12(s, 2H); 4.20 |
| | | (brs, 2H); 3.33(brs, |
| | | 2H); 2.78(brs, 2H); |
| | | 1.9-1.6(m, 3H); 1.3- |
| | | 1.0(m, 2H). |
| 10. | 4-[(2-Fluoro-4-hydroxy-benzoylamino)-methyl]- | M.S(M+1): 387 |
| | piperidine-1-carboxylic acid benzyl ester | NMR(300 MHz, |
| | | CDCl₃))δ: 7.94(t, |
| | | 1H, J=9.0 Hz); 7.5- |
| | | 7.2(m, 5H); 6.78 |
| | | (m, 1H); 7.73(dd, |
| | | 1H, J=8.7 and 2.4 |
| | | Hz); 6.61(dd, 1H, |
| | | J=13.8 and 2.2 Hz); |
| | | 5.13(s, 2H); 4.20 |
| | | (brs, 2H); 3.37(brs, |
| | | 2H); 2.78(brs, 2H); |
| | | 1.9-1.6(m, 3H); 1.3- |
| | | 1.0(m, 2H). |
| 11. | 4-{[(1H-Benzoimidazole-5-carbonyl)- | MS Exact mass: |
| | amino]-methyl}-piperidine-1-carboxylic | 393.1940. |
| | acid benzyl ester | Experimental for |
| | | C₂₁H₂₄N₄O₃: |
| | | 393.1921.¹H NMR |
| | | (400 MHz, δ, |
| | | CDCl₃): 8.13-8.11 |
| | | (m, 2H), 7.67(brs, |
| | | 2H), 7.35-7.28(m, |
| | | 5H), 6.52(d, |
| | | J=5.98 Hz, 2H), |
| | | 5.13(s, 2H), 4.21 |
| | | (brs, 2H), 3.39 |
| | | (brs, 2H), 2.79 |
| | | (brs, 2H), 1.90- |
| | | 1.78(m, 1H), |
| | | 1.78-1.62(m, 2H), |
| | | 1.29-1.16(m, 2H). |
| 12. | 4-{[(1H-Benzotriazole-5-carbonyl)- | MS Exact mass: |
| | amino]-methyl}-piperidine-1-carboxylic | 394.1896. |
| | acid benzyl ester | Experimental for |
| | | C₂₁H₂₃N₅O₃: |
| | | 394.1874. ¹H NMR |
| | | (400 MHz, δ, |
| | | CDCl₃): 8.37(s, |
| | | 1H), 7.78(d, |
| | | J=8.68 Hz, 2H), |
| | | 7.66-7.64(m, 2H), |
| | | 7.31-7.22(m, 5H), |
| | | 6.65(vbs, 2H), |
| | | 5.09(s, 2H), 4.13 |
| | | (brd, J=11.06, |
| | | 2H), 3.35(brs, |
| | | 2H), 2.71(brs, |
| | | 2H), 1.90-1.77(m, |
| | | 1H), 1.71(brd, |
| | | J=11.61 Hz, 2H), |
| | | 1.26-1.12(m, 2H). |
| 13. | 4-[(4-Cyano-benzoylamino)-methyl]- | ¹H NMR(δ, |
| | piperidine-1-carboxylic acid benzyl ester | CDCl₃): 7.86(d, |
| | | J=8.05 Hz, 2H), |
| | | 7.74(d, J=8.05 Hz, |
| | | 2H), 7.25-7.4(m, |
| | | 5H), 6.31(brt, J= |
| | | 5.61 Hz, 1H), 5.12 |
| | | (s, 2H), 4.22(brs, |
| | | 2H), 3.37(brs, |
| | | 2H), 2.79(brs, |
| | | 2H), 1.7-1.9(m, |
| | | 3H), 1.23(m, 2H). |
| 14. | 4-{[(6-Hydroxy-pyridine-3-carbonyl)-amino]- | M.S(M+1): 384 |
| | methyl}-piperidine-1-carboxylic acid 4-methyl- | NMR(300 MHz, |
| | benzyl ester | CDCl₃)δ: 12.20 |
| | | (brs, 1H); 8.02(d, |
| | | 1H, J=2.5 Hz); 7.75 |
| | | (dd, 1H, J=9.6 and |
| | | 2.5 Hz); 7.24(d, 2H, |
| | | J=7.9 Hz); 7.15(d, |
| | | 2H, J=7.9 Hz); 6.56 |
| | | (d, 1H, J=9.6Hz); |
| | | 6.20(m, 1H); 5.07 |
| | | (s, 2H); 4.20(brs, |
| | | 2H); 3.30(brs, 2H); |
| | | 2.35(brs, 2H); 1.8- |
| | | 1.6(m, 3H); 1.3-1.1 |
| | | (m, 2H). |
| 15. | 4-{[(6-Hydroxy-pyridazine-3-carbonyl)-amino]- | M.S(M+1): 385 |
| | methyl}-piperidine-1-carboxylic acid 4-methyl- | NMR(300 MHz, |
| | benzyl ester | CDCl₃))δ: 11.9(s, |
| | | 1H); 8.05(d, 1H, |
| | | J=9.9 Hz); 7.25(d, |
| | | 2H, J=7.9 Hz); 7.16 |
| | | (d, 2H, J=7.9 Hz); |
| | | 7.04(d, 1H, J=9.9 |
| | | Hz); 5.08(s, 2H); |
| | | 4.20(brs, 2H); 3.32 |
| | | (brs, 2H); 2.76(m, |
| | | 2H); 2.35(s, 3H); |
| | | 1.8-1.6(m, 3H); 1.3- |
| | | 1.1(m, 2H). |
| 16. | 4-{[(6-Hydroxy-pyridine-3-carbonyl)-amino]- | M.S(M+1): 388 |
| | methyl}-piperidine-1-carboxylic acid 4-fluoro- | NMR(300 MHz, |
| | benzyl ester | CDCl₃))δ: 12.2(s, |
| | | 1H); 8.03(d, 1H, |
| | | J=2.6 Hz); 7.77(dd, |
| | | 1H, J=9.6 and 2.6 |
| | | Hz); 7.34(m, 2H); |
| | | 7.03(t, 2H, J=8.6 |
| | | Hz); 6.57(d, 1H, |
| | | J=9.6 Hz); 5.07(s, |
| | | 2H); 4.20(brs, 2H); |
| | | 3.31(brs, 2H); 2.76 |
| | | (brs, 2H); 2.35(s, |
| | | 3H); 1.8-1.6(m, |
| | | 3H); 1.3-1.1(m, |
| | | 2H). |
| 17. | 4-{[(6-Hydroxy-pyridine-3-carbonyl)-amino]- | M.S(M+1): 404 |
| | methyl}-piperidine-1-carboxylic acid 4-chloro- | NMR(300 MHz, |
| | benzyl ester | CDCl₃))δ: 11.8 |
| | | (brs, 1H); 8.02(d, |
| | | 1H, J=2.4 Hz); 7.74 |
| | | (dd, 1H, J=9.6 and |
| | | 2.4 Hz); 7.4-7.2(m, |
| | | 4H); 6.58(d, 1H, |
| | | J=9.6 Hz); 6.03(m, |
| | | 1H); 5.08(s, 2H); |
| | | 4.20(brs, 2H); 3.31 |
| | | (brs, 2H); 2.78(brs, |
| | | 2H); 1.8-1.4(m, |
| | | 3H); 1.3-1.1(m, 2H). |
| 18. | 4-{[(6-Hydroxy-pyridine-3-carbonyl)-amino]- | M.S(M+1): 396 |
| | methyl}-piperidine-1-carboxylic acid indan-2-yl ester | NMR(300 MHz, |
| | | CDCl₃))δ: 12.0 |
| | | (brs, 1H); 8.01(d, |
| | | 1H, J=2.5 Hz); 7.74 |
| | | (dd, 1H, J=9.6 and |
| | | 2.5 Hz); 7.3-7.1(m, |
| | | 4H); 6.57(d, 1H, |
| | | J=9.6 Hz); 6.04(m, |
| | | 1H); 5.46(m, 1H); |
| | | 4.3-4.1(m, 2H); |
| | | 3.32(m, 4H); 3.04 |
| | | (d, 1H, J=3.2 Hz); |
| | | 3.00(d, 1H, J=3.2 |
| | | Hz); 2.72(m, 2H); |
| | | 1.8-1.6(m, 3H); |
| | | 1.3-1.0(m, 2H). |
| 19. | 4-[(4-Hydroxy-benzoylamino)-methyl]- | M.S(M+1): |
| | piperidine-1-carboxylic acid 4-fluoro-benzyl ester | 387NMR(300 MHz, |
| | | CDCl₃))δ: 7.65 |
| | | (d, 2H, J=8.6 Hz); |
| | | 7.33(m, 2H); 7.03 |
| | | (t, 2H, J=8.6 Hz); |
| | | 6.86(d, 2H, J=8.6 |
| | | Hz); 6.64(s, 1H); |
| | | 6.22(m, 1H); 5.08(s, |
| | | 2H); 4.14(brs, 2H); |
| | | 3.33(brs, 2H); 2.67 |
| | | (brs, 2H); 1.8-1.6 |
| | | (m, 3H); 1.3-1.0(m, |
| | | 2H). |
| 20. | 4-[(4-Hydroxy-benzoylamino)-methyl]- | M.S(M+1): 403 |
| | piperidine-1-carboxylic acid 4-chloro-benzyl ester | NMR(300 MHz, |
| | | CDCl₃))δ: 7.66(d, |
| | | 2H, J=8.6 Hz); 7.30 |
| | | (m, 4H); 6.86(d, |
| | | 2H, J=8.6 Hz); 6.33 |
| | | (s, 1H); 6.22(m, |
| | | 1H); 5.08(s, 2H); |
| | | 4.14(brs, 2H); 3.33 |
| | | (brs, 2H); 2.77(brs, |
| | | 2H); 1.8-1.6(m, |
| | | 3H); 1.3-1.0(m, |
| | | 2H). |
| 21. | 4-[(4-Hydroxy-benzoylamino)-methyl]- | M.S(M+1): |
| | piperidine-1-carboxylic acid indan-2-yl ester | 395NMR(300 MHz, |
| | | CDCl₃))δ: 7.63(d, |
| | | 2H, J=8.6 Hz); 7.3- |
| | | 7.1(m, 4H); 6.85 |
| | | (d, 2H, J=8.6 Hz); |
| | | 6.27(m, 1H); 5.46 |
| | | (m, 1H); 4.3-3.8(m, |
| | | 2H); 3.3(dd, 4H, |
| | | J=16.9 and 6.6 Hz); |
| | | 3.0(dd, 2H, J=7.0 |
| | | and 3.2 Hz); 2.69 |
| | | (dt, 2H, J=13.2 and |
| | | 2.7 Hz); 1.8-1.6(m, |
| | | 3H); 1.3-1.0(m, |
| | | 2H). |
| 22. | 4-[(4-Hydroxy-benzoylamino)-methyl]- | M.S(M+1): 383 |
| | piperidine-1-carboxylic acid 4-methyl-benzyl | NMR(300 MHz, |
| | ester | CDCl₃))δ: 7.64(d, |
| | | 2H, J=8.8 Hz); 7.24 |
| | | (d, 1H, J=8.0 Hz); |
| | | 7.15(d, 1H, J=8.0 |
| | | Hz); 6.86(d, 2H, |
| | | J=8.8 Hz); 6.24(m, |
| | | 1H); 5.08(s, 2H); |
| | | 4.18(brs, 2H); 3.32 |
| | | (brs, 2H); 2.75(brs, |
| | | 2H); 2.34(s, 3H); |
| | | 1.8-1.6(m, 3H); |
| | | 1.3-1.0(m, 2H). |
| 23. | 4-{[(Pyridine-4-carbonyl)-amino]- | ¹H NMR(δ, |
| | methyl}-piperidine-1-carboxylic acid 4- | CDCl₃): 8.75(d, |
| | methyl-benzyl ester | J=5.86Hz, 2H), |
| | | 7.60(d, J=4.89 |
| | | Hz, 2H), 7.25(d, |
| | | J=8.05 Hz, 2H), |
| | | 7.16(d, J=8.05Hz, |
| | | 2H), 6.32(brt, |
| | | 1H), 5.08(s, 2H), |
| | | 4.22(brs, 2H), |
| | | 3.37(brs, 2H), |
| | | 2.77(brs, 2H), |
| | | 2.35(s, 3H), 1.7- |
| | | 1.9(m, 3H), 1.21 |
| | | (m, 2H). |
| 24. | 4-{[(Pyridine-4-carbonyl)-amino]- | ¹H NMR(δ, |
| | methyl}-piperidine-1-carboxylic acid 4- | CDCl₃): 8.75(d, |
| | chloro-benzyl ester | J=4.64 Hz, 2H), |
| | | 7.60(d, J=5.13 |
| | | Hz, 2H), 7.32(d, |
| | | J=8.05 Hz, 2H), |
| | | 7.28(d, J=8.55 |
| | | Hz, 2H), 6.35(brt, |
| | | 1H), 5.08(s,2H), |
| | | 4.22(brd, 2H), |
| | | 3.37(brd, 2H), |
| | | 2.79(brs, 2H), |
| | | 1.7-1.9(m, 3H), |
| | | 1.23(m, 2H). |
| 25. | 4-{[(Pyridine-4-carbonyl)-amino]- | ¹H NMR(δ, |
| | methyl}-piperidine-1-carboxylic acid 4- | CDCl₃): 8.75(d, |
| | fluoro-benzyl ester | J=5.61 Hz, 2H), |
| | | 7.60(d, J=6.11 |
| | | Hz, 2H), 7.28(dd, |
| | | J=5.62, 8.3 Hz, |
| | | 2H), 7.04(t, J=8.8 |
| | | Hz, 2H), 6.33(brt, |
| | | 1H), 5.08(s, 2H), |
| | | 4.23(brd, 2H), |
| | | 3.38(brd, 2H), |
| | | 2.78(brs, 2H), |
| | | 1.7-1.9(m, 3H), |
| | | 1.22(m, 2H). |
| 26. | 4-[(4-Hydroxy-3-methyl-benzoylamino)- | ¹H NMR(δ, |
| | methyl]-piperidine-1-carboxylic acid | CDCl₃): 7.56(brs, |
| | benzyl ester | 1H), 7.49(dd, |
| | | J=2.2, 8.3 Hz, |
| | | 1H), 7.25-7.4(m, |
| | | 5H), 6.79(d, |
| | | J=8.3 Hz, 1H), |
| | | 6.13(brt, 1H), |
| | | 5.55(s, 1H), 5.12 |
| | | (s, 2H), 4.22(brs, |
| | | 2H), 3.33(brs, |
| | | 2H), 2.78(brs, |
| | | 2H), 2.28(s, 3H), |
| | | 1.7-1.9(m, 3H), |
| | | 1.23(m, 2H). |
| 27. | 4-[(3-Chloro-4-hydroxy-benzoylamino)- | ¹H NMR(δ, |
| | methyl]-piperidine-1-carboxylic acid | CDCl₃): 7.80(d, |
| | benzyl ester | J=2.2 Hz, 1H), |
| | | 7.57(dd, J=2.2, |
| | | 8.55 Hz, 1H), |
| | | 7.25-7.4(m, 5H), |
| | | 7.05(d, J=8.55 |
| | | Hz, 1H), 6.13(brt, |
| | | 1H), 6.04 |
| | | (brs, 1H), 5.12 |
| | | (s, 2H), 4.22(brs, |
| | | 2H), 3.33(brs, |
| | | 2H), 2.78(brs, |
| | | 2H), 1.7-1.9(m, |
| | | 3H), 1.23(m, 2H). |
| 28. | 4-{[(Thiophene-3-carbonyl)-amino]- | ¹H NMR(δ, |
| | methyl}-piperidine-1-carboxylic acid | CDCl₃): 7.84(s, |
| | benzyl ester | 1H), 7.41-7.27(m, |
| | | 7H), 6.24(brt, |
| | | 1H), 5.06(s, 2H), |
| | | 4.19(brd, 2H), |
| | | 3.30(brs, 2H), |
| | | 2.77(brt, 2H), |
| | | 1.9-1.7(m, 3H), |
| | | 1.18(m, 2H). |
| 29. | 4-{[(Thiazole-4-carbonyl)-amino]- | ¹H NMR(δ, |
| | methyl}-piperidine-1-carboxylic acid | CDCl₃): 8.74(d, |
| | benzyl ester | 1H), 8.17(d, 1H), |
| | | 7.50(brt, 1H), |
| | | 7.26(m, 5H), 5.11 |
| | | (s, 2H), 4.19(brs, |
| | | 2H), 3.35(brs, |
| | | 2H), 2.78(brt, |
| | | 2H), 1.9-1.7(m, |
| | | 3H), 1.21(m, 2H). |
| 30. | 4-{[(2H-Pyrazole-3-carbonyl)-amino]- | ¹H NMR(δ, |
| | methyl}-piperidine-1-carboxylic acid | CDCl₃): 7.59(d, |
| | benzyl ester | J=1.3Hz, 1H), |
| | | 7.36-7.28(m, 5H), |
| | | 7.07(brt, 1H), |
| | | 6.82(d, J=1.3Hz, |
| | | 1H), 5.13(s, 2H), |
| | | 4.20(brs, 2H), |
| | | 3.37(brs, 2H), |
| | | 2.78(brt, 2H), |
| | | 1.9-1.7(m, 3H), |
| | | 1.21(m, 2H). |
| 31. | 4-{[(5-Oxo-4,5-dihydro-1H-[1,2,4]triazole- | ¹H NMR |
| | 3-carbonyl)-amino]-methyl}-piperidine- | (500 MHz, δ, |
| | 1-carboxylic acid benzyl ester | CDCl₃): 11.55(s, |
| | | br, 2H), 7.45-7.30 |
| | | (m, 6H), 5.12(s, |
| | | 2H), 4.19(s, 2H), |
| | | 3.25(m, 2H), 2.75 |
| | | (m, 2H), 1.85-1.65 |
| | | (m, 3H), 1.15(m, |
| | | 2H). |
| 32. | 4-{[(2H-[1,2,4]Triazole-3-carbonyl)- | ¹H NMR |
| | amino]-methyl}-piperidine-1-carboxylic | (500 MHz, δ, |
| | acid benzyl ester | DMSO-d₆): 14.60 |
| | | (s, br, 1H), 8.80- |
| | | 8.30(s, br, 2H), |
| | | 7.40-7.30(m, 5H), |
| | | 5.07(s, 2H), 3.98 |
| | | (d, 2H), 3.15(t, |
| | | 2H), 2.77(m, br, |
| | | 2H), 1.77(m, 1H), |
| | | 1.63(d, 2H), 1.05 |
| | | (m, 2H). |
| 33. | 4-{[(Thiazole-5-carbonyl)-amino]- | ¹H NMR |
| | methyl}-piperidine-1-carboxylic acid | (500 MHz, δ, |
| | benzyl ester | DMSO-d₆): 9.21 |
| | | (s, 1H), 8.74(m, |
| | | 1H), 8.46(s, 1H), |
| | | 7.40-7.28(m, 5H), |
| | | 5.09(s, 2H), 4.00 |
| | | (d, 2H), 3.12(t, |
| | | 2H), 2.90-2.70(m, |
| | | br, 2H), 1.80-1.65 |
| | | (m, 3H), 1.05(m, |
| | | 2H). |
| 34. | 4-{[(1H-Pyrazole-4-carbonyl)-amino]- | ¹H NMR |
| | methyl}-piperidine-1-carboxylic acid | (500 MHz, δ, |
| | benzyl ester | CD₃OD): 8.2-7.8 |
| | | (s, br, 2H), 7.36- |
| | | 7.25(m, 5H), 5.11 |
| | | (s, 2H), 4.15(m, |
| | | 2H), 3.23(m, 2H), |
| | | 2.90-2.75(s, br, |
| | | 2H), 1.90-1.70(m, |
| | | 3H), 1.20-1.10(m, |
| | | 2H). |
| 35. | 4-{[(2-Bromo-pyridine-4-carbonyl)- | ¹H NMR |
| | amino]-methyl}-piperidine-1-carboxylic | (500 MHz, δ, |
| | acid benzyl ester | DMSO-d₆): 8.88 |
| | | (m, 1H), 8.54(d, |
| | | 1H), 7.99(s, 1H), |
| | | 7.78(d, 1H), 7.38- |
| | | 7.28(m, 5H), 5.07 |
| | | (s, 1H), 4.00(d, |
| | | 2H), 3.16(t, 2H), |
| | | 2.90-2.70(m, 2H), |
| | | 1.80-1.65(m, 3H), |
| | | 1.09(m, 2H). |
| 36. | 4-{[(1H-Pyrrole-2-carbonyl)-amino]- | ¹H NMR(δ, |
| | methyl}-piperidine-1-carboxylic acid | CDCl₃): 9.55(brs, |
| | benzyl ester | 1H), 7.39-7.28(m, |
| | | 5H), 6.92(m, 1H), |
| | | 6.57(m, 1H), 6.22 |
| | | (m, 1H), 6.01(brt, |
| | | 1H), 5.08(s, 2H), |
| | | 4.20(brs, 2H), |
| | | 3.28(brs, 2H), |
| | | 2.77(brt, 2H), |
| | | 1.9-1.7(m, 3H), |
| | | 1.21(m, 2H). |
| 37. | 4-{[(1H-Imidazole-4-carbonyl)-amino]- | ¹H NMR(δ, |
| | methyl}-piperidine-1-carboxylic acid | CDCl₃): 7.58(m, |
| | benzyl ester | 2H), 7.38-7.27(m, |
| | | 5H), 5.10(s, 2H), |
| | | 4.20(brd, 2H), |
| | | 3.37(brs,2H), |
| | | 2.77(brt, 2H), |
| | | 1.9-1.7(m, 3H), |
| | | 1.21(m, 2H). |
| 38. | 4-{[(1-Methyl-1H-pyrrole-2-carbonyl)- | ¹H NMR(δ, |
| | amino]-methyl}-piperidine-1-carboxylic | CDCl₃): 7.38-7.25 |
| | acid benzyl ester | (m, 5H), 6.71(m, |
| | | 1H), 6.50(m, 1H), |
| | | 6.08(m, 1H), 6.00 |
| | | (brt, 1H), 5.11(s, |
| | | 2H), 4.22(brs, |
| | | 2H), 3.94(s, 3H), |
| | | 3.26(brs, 2H), |
| | | 2.77(brt, 2H), |
| | | 1.9-1.7(m, 3H), |
| | | 1.21(m, 2H). |
| 39. | 4-{[(5-Methyl-3H-imidazole-4-carbonyl)- | ¹H NMR(δ, |
| | amino]-methyl}-piperidine-1-carboxylic | CDCl₃): 9.62(brs, |
| | acid benzyl ester | 1H), 7.40(s, 1H), |
| | | 7.31(m, 6H), 5.12 |
| | | (s, 2H), 4.19(brd, |
| | | 2H), 3.25(brs, |
| | | 2H), 2.77(brt, |
| | | 2H), 2.59(s, 3H), |
| | | 1.9-1.7(m, 3H), |
| | | 1.21(m, 2H). |
| 40. | 4-{[(1H-Pyrrole-3-carbonyl)-amino]- | ¹H NMR(δ, |
| | methyl}-piperidine-1-carboxylic acid | CDCl₃): 8.55(brs, |
| | benzyl ester | 1H), 7.28(m, 6H), |
| | | 6.78(s, 1H), 6.40 |
| | | (s, 1H), 5.88(brt, |
| | | 1H), 5.10(s, 2H), |
| | | 4.19(brs, 2H), |
| | | 3.30(brs, 2H), |
| | | 2.77(brt, 2H), |
| | | 1.9-1.7(m, 3H), |
| | | 1.20(m, 2H). |
| 41. | 4-{[(Thiophene-3-carbonyl)-amino]- | ¹H NMR(δ, |
| | methyl}-piperidine-1-carboxylic acid 4- | CDCl₃): 7.83(m, |
| | methyl-benzyl ester | 1H), 7.38(m, 2H), |
| | | 7.24(d, 2H), 7.18 |
| | | (d, 2H), 6.19(brt, |
| | | 1H), 5.02(s, 2H), |
| | | 4.20(bra, 2H), |
| | | 3.30(brs, 2H), |
| | | 2.77(brt, 2H), |
| | | 2.35(s, 3H), 1.9- |
| | | 1.7(m, 3H), 1.21 |
| | | (m, 2H). |
| 42. | 4-{ [(2H-Pyrazole-3-carbonyl)-amino]- | ¹H NMR(δ, |
| | methyl}-piperidine-1-carboxylic acid 4- | CDCl₃): 7.60(d, |
| | fluoro-benzyl ester | 1H), 7.30(d, 2H), |
| | | 7.04(m, 3H), 6.82 |
| | | (d, 1H), 5.04(s, |
| | | 2H), 4.18(brs, |
| | | 2H), 3.33(brs, |
| | | 2H), 2.77(brt, |
| | | 2H), 1.9-1.7(m, |
| | | 3H), 1.21(m, 2H). |
| 43. | 4-{[(2H-Pyrazole-3-carbonyl)-amino]- | ¹H NMR(δ, |
| | methyl}-piperidine-1-carboxylic acid 4- | CDCl₃): 7.58(d, |
| | chloro-benzyl ester | 1H), 7.27(m, 4H), |
| | | 7.04(brt, 1H), |
| | | 6.82(d, 1H), 5.05 |
| | | (s, 2H), 4.18(brs, |
| | | 2H), 3.36(brs, |
| | | 2H), 2.77(brt, |
| | | 2H), 1.9-1.7(m, |
| | | 3H), 1.21(m, 2H). |
| 44. | 4-{[(2H-Pyrazole-3-carbonyl)-amino]- | ¹H NMR(δ, |
| | methyl}-piperidine-1-carboxylic acid 4- | CDCl₃): 7.60(d, |
| | methyl-benzyl ester | 1H), 7.22(d, 2H), |
| | | 7.17 d, 2H), 6.97 |
| | | (brt, 1H), 6.84(d, |
| | | 1H),, 5.04(s, 2H), |
| | | 4.20(brs, 2H), |
| | | 3.35(brs, 2H), |
| | | 2.77(brt, 2H), |
| | | 2.37(m, 3H), 1.9- |
| | | 1.7(m, 3H), 1.21 |
| | | (m, 2H). |
| 45. | 4-{[(1H-Pyrazole-4-carbonyl)-amino]- | ¹H NMR(δ, |
| | methyl}-piperidine-1-carboxylic acid 4- | CDCl₃): 7.94(s, |
| | fluoro-benzyl ester | 2H), 7.30(m, 2H), |
| | | 7.01(m, 2H), 6.60 |
| | | (brs, 1H), 5.03(s, |
| | | 2H), 4.16(brd, |
| | | 2H), 3.24(brs, |
| | | 2H), 2.75(brs, |
| | | 2H), 1.9-1.7(m, |
| | | 3H), 1.15(m, 2H). |
| 46. | 4-{[(1H-Pyrazole-4-carbonyl)-amino]- | ¹H NMR(δ, |
| | methyl}-piperidine-1-carboxylic acid 4- | CDCl₃): 7.94(s, |
| | chloro-benzyl ester | 2H), 7.26(m, 4H), |
| | | 6.43(brs, 1H), |
| | | 5.03(s, 2H), 4.17 |
| | | (brs, 2H), 3.25 |
| | | (brs, 2H), 2.77 |
| | | (brs, 2H), 1.9-1.7 |
| | | (m, 3H), 1.15(m, 2H). |
| 47. | 4-{[(1H-Pyrazole-4-carbonyl)-amino]- | ¹H NMR(δ, |
| | methyl}-piperidine-1-carboxylic acid 4- | CDCl₃): 7.94(s, |
| | methyl-benzyl ester | 2H), 7.25(d, 2H), |
| | | 7.16(d, 2H), 6.03 |
| | | (brt, 1H), 5.06(s, |
| | | 2H), 4.20(brs, |
| | | 2H), 3.30(brs, |
| | | 2H), 2.77(brt, |
| | | 2H), 2.37(s, 3H), |
| | | 1.9-1.7(m, 3H), |
| | | 1.20(m, 2H). |
| 48. | 4-{[(1H-Pyrazole-4-carbonyl)-amino]- | ¹H NMR(δ, |
| | methyl}-piperidine-1-carboxylic acid | CDCl₃): 7.94(s, |
| | indan-2-yl ester | 2H), 7.20(m, 4H), |
| | | 6.15(brt, 1H), |
| | | 5.42(m,1H), 4.10 |
| | | (brd, 2H), 3.30(m, |
| | | 4H), 3.00(dd, |
| | | 2H), 2.70(t, 2H), |
| | | 1.8-1.6(m, 3H), |
| | | 1.18(m, 2H). |
| 49. | 4-{[(1H-Pyrrole-2-carbonyl)-amino]- | ¹H NMR(δ, |
| | methyl}-piperidine-1-carboxylic acid 4- | CDCl₃): 9.43(brs, |
| | methyl-benzyl ester | 1H), 7.24(d, 2H), |
| | | 7.17(d, 2H), 6.91 |
| | | (s, 1H), 6.55(s, |
| | | 1H), 6.22(m, 1H), |
| | | 5.95(brt, 1H), |
| | | 5.06(s, 2H), 4.19 |
| | | (brs, 2H), 3.30 |
| | | (brs, 2H), 2.77 |
| | | (brt, 2H), 2.36(s, |
| | | 3H), 1.9-1.7(m, |
| | | 3H), 1.18(m, 2H). |
| 50. | 4-{[(1H-Imidazole-4-carbonyl)-amino]- | ¹H NMR(δ, |
| | methyl}-piperidine-1-carboxylic acid 4- | CDCl₃): 7.59(s, |
| | chloro-benzyl ester | 2H), 7.30(m, 5H), |
| | | 5.06(s, 2H), 4.18 |
| | | (brs, 2H), 3.33 |
| | | (brs, 2H), 2.77 |
| | | (brt, 2H), 1.9-1.7 |
| | | (m, 3H), 1.21(m, |
| | | 2H). |
| 51. | 4-{[(1-Methyl-1H-pyrrole-2-carbonyl)- | ¹H NMR(δ, |
| | amino]-methyl}-piperidine-1-carboxylic | CDCl₃): 7.31(dd, |
| | acid 4-fluoro-benzyl ester | 2H), 7.02(dd, |
| | | 2H), 6.72(s, 1H), |
| | | 6.50(m, 1H), 6.08 |
| | | (m, 1H), 6.00(brt, |
| | | 1 H), 5.04(s, 2H), |
| | | 4.18(brs, 2H), |
| | | 3.93(s, 3H), 3.25 |
| | | (brs, 2H), 2.77 |
| | | (brt, 2H), 1.9-1.7 |
| | | (m, 3H), 1.18(m, |
| | | 2H). |
| 52. | 4-{[(1H-Pyrrole-2-carbonyl)-amino]- | ¹H NMR(δ, |
| | methyl}-piperidine-1-carboxylic acid 4- | CDCl₃): 9.37(brs, |
| | chloro-benzyl ester | 1H), 7.24(m, 4H), |
| | | 6.92(s, 1H), 6.53 |
| | | (s, 1H), 6.22(m, |
| | | 1H), 5.93(brt, |
| | | 1H), 5.06(s, 2H), |
| | | 4.20(brs, 2H), |
| | | 3.31(brs, 2H), |
| | | 2.77(brt, 2H), |
| | | 1.9-1.7(m, 3H), |
| | | 1.18(m, 2H). |
| 53. | 4-{[(2-Methyl-1H-pyrrole-3-carbonyl)- | ¹H NMR(δ, |
| | amino]-methyl}-piperidine-1-carboxylic | CDCl₃): 8.10(brs, |
| | acid 4-methyl-benzyl ester | 1H), 7.25(d, 2H), |
| | | 7.17(d, 2H), 6.59 |
| | | (m, 1H), 6.23(m, |
| | | 1H), 5.81(brt, |
| | | 1H), 5.06(s, 2H), |
| | | 4.20(brs, 2H), |
| | | 3.26(brs, 2H), |
| | | 2.77(brt, 2H), |
| | | 2.55(s, 3H), 2.36 |
| | | (s, 3H), 1.9-1.7 |
| | | (m, 3H), 1.20(m, |
| | | 2H). |
| 54. | 4-{[(1H-Pyrrole-3-carbonyl)-amino]- | ¹H NMR(δ, |
| | methyl}-piperidine-1-carboxylic acid 4- | CDCl₃): 8.55(brs, |
| | methyl-benzyl ester | 1H), 7.36(m, 1H), |
| | | 7.25(d, 2H), 7.17 |
| | | (d, 2H), 6.77(m, |
| | | 1H), 6.40(m, 1H), |
| | | 5.86(brt, 1H), |
| | | 5.06(s, 2H), 4.19 |
| | | (brs, 2H), 3.29 |
| | | (brs, 2H), 2.77 |
| | | (brt, 2H), 2.36(s, |
| | | 3H), 1.9-1.7(m, |
| | | 3H), 1.18(m, 2H). |
| 55. | 4-{[(Thiazole-5-carbonyl)-amino]- | ¹H NMR(δ, |
| | methyl}-piperidine-1-carboxylic acid 4- | CDCl₃): 8.90(s, |
| | methyl-benzyl ester | 1H), 8.24(s, 1H), |
| | | 7.24(d, 2H), 7.16 |
| | | (d, 2H), 6.24(brt, |
| | | 1H), 5.05(s, 2H), |
| | | 4.20(brs, 2H), |
| | | 3.35(brs, 2H), |
| | | 2.77(brt, 2H), |
| | | 2.36(s, 3H), 1.9- |
| | | 1.7(m, 3H), 1.21 |
| | | (m, 2H). |
| 56. | 4-{[(Oxazole-5-carbonyl)-amino]-methyl}- | ¹H NMR(δ, |
| | piperidine-1-carboxylic acid 4-methyl-benzyl ester | CDCl₃): 7.90(s, |
| | | 1H), 7.72(s, 1H), |
| | | 7.23(d, 2H), 7.17 |
| | | (d, 2H), 6.35(brt, |
| | | 1H), 5.05(s, 2H), |
| | | 4.20(brs, 2H), |
| | | 3.33(brs, 2H), |
| | | 2.77(brt, 2H), |
| | | 2.35(s, 3H), 1.9- |
| | | 1.7(m, 3H), 1.20 |
| | | (m, 2H). |
| 57. | 4-{[(1H-Pyrazole-4-carbonyl)-amino]- | ¹H NMR(δ, |
| | methyl}-piperidine-1-carboxylic acid 4- | CDCl₃): 7.93(s, |
| | isopropyl-benzyl ester | 2H), 7.25(m, 4H), |
| | | 6.62(brt, 1H), |
| | | 5.07(s, 2H), 4.16 |
| | | (brd, 2H), 3.26 |
| | | (brs, 2H), 2.89(m, |
| | | 1H), 2.71(brt, |
| | | 2H), 1.9-1.7(m, |
| | | 3H), 1.23(d, 6H), |
| | | 1.18(m, 2H). |
| 58. | 4-{[(1H-Pyrazole-4-carbonyl)-amino]- | ¹H NMR(δ, |
| | methyl}-piperidine-1-carboxylic acid | CDCl₃): 10.50 |
| | thiophen-3-ylmethyl ester | (brs, 1H), 7.94(s, |
| | | 2H), 7.28(m, 2H), |
| | | 7.08(m, 1H), 5.93 |
| | | (brt, 1H), 5.11(s, |
| | | 2H), 4.19(brs, |
| | | 2H), 3.31(brs, |
| | | 2H), 2.77(brt, |
| | | 2H), 1.9-1.7(m, |
| | | 3H), 1.19(m, 2H). |
| 59. | 4-[(4-Hydroxy-benzoylamino)-methyl]- | ¹H NMR(δ, |
| | piperidine-1-carboxylic acid 4- | CD₃OD): 8.24 |
| | isopropyl-benzyl ester | (brd, 1H) 7.68(d, |
| | | 2H), 7.20(m, 4H), |
| | | 6.79(d, 2H), 5.02 |
| | | (s, 2H), 4.10(d, |
| | | 2H), 3.20(t, 2H), |
| | | 2.81(m, 1H), 2.77 |
| | | (brs, 2H), 1.77(m, |
| | | 1H), 1.70(brd, |
| | | 2H), 1.20(d, 6H), |
| | | 1.16(m, 2H). |
| 60. | 4-[(4-Hydroxy-benzoylamino)-methyl]- | ¹H NMR(δ, |
| | piperidine-1-carboxylic acid thiophen- | CDCl₃): 7.94(s, |
| | 3-ylmethyl ester | 2H), 7.26(m, 4H), |
| | | 7.09(d, 1H), 5.92 |
| | | (brt, 1H), 5.14(s, |
| | | 2H), 4.19(brs, |
| | | 2H), 3.30(brs, |
| | | 2H), 2.77(brt, |
| | | 2H), 1.9-1.7(m, |
| | | 3H), 1.20(m, 2H). |
| 61. | 4-{[(Pyridine-4-carbonyl)-amino]- | ¹H NMR(δ, |
| | methyl}-piperidine-1-carboxylic acid 4- | CDCl₃): 8.72(d, |
| | isopropyl-benzyl ester | 2H), 7.60(d, 2H), |
| | | 7.22(m, 4H), 6.55 |
| | | (brt, 1H), 5.06(s, |
| | | 2H), 4.21(brd, |
| | | 2H), 3.33(brs, |
| | | 2H), 2.90(m, 1H), |
| | | 2.77(brt, 2H), |
| | | 1.9-1.7(m, 3H), |
| | | 1.21(d, 6H), 1.18 |
| | | (m, 2H). |
| 62. | 4-{[(2H-Pyrazole-3-carbonyl)-amino]- | ¹H NMR(δ, |
| | methyl}-piperidine-1-carboxylic acid 4- | CDCl₃): 7.57(m, |
| | isopropyl-benzyl ester | 1H), 7.23(m, |
| | | 4H), 7.02(brt, |
| | | 1H), 6.83(m, 1H), |
| | | 5.06(s, 2H), 4.19 |
| | | (brs, 2H), 3.33 |
| | | (brs, 2H), 2.90(m, |
| | | 1H), 2.77(brt, |
| | | 2H), 1.9-1.7(m, |
| | | 3H), 1.21(d, 6H), |
| | | 1.18(m, 2H). |
| 63. | 4-{[(1H-Pyrrole-3-carbonyl)-amino]- | ¹H NMR(δ, |
| | methyl}-piperidine-1-carboxylic acid 4- | CDCl₃): 9.79(brs, |
| | isopropyl-benzyl ester | 1H), 7.30-7.15(m, |
| | | 5H), 6.70(s, 1H), |
| | | 6.42(s, 1H), 6.30 |
| | | (brt, 1H), 5.06(s, |
| | | 2H), 4.17(brs, |
| | | 2H), 3.25(brs, |
| | | 2H), 2.90(m, 1H), |
| | | 2.75(brs, 2H), |
| | | 1.9-1.7(m, 3H), |
| | | 1.22(d, 6H), 1.17 |
| | | (m, 2H). |
| 64. | 4-Hydroxy-N-[1-(3-phenyl-propionyl)- | ¹H NMR(δ, |
| | piperidin-4-ylmethyl]-benzamide | CDCl₃): 8.80(brs, |
| | | 1H), 7.63(d, 2H), |
| | | 7.3-7.1(m, 5H), |
| | | 6.89(d, 2H), 6.69 |
| | | (brt, 1H), 4.58(d, |
| | | 1H), 3.76(d, 1H), |
| | | 3.35-3.18(m, 2H), |
| | | 2.90(m, 3H), 2.60 |
| | | (t, 2H), 2.49(t, |
| | | 1H), 1.9-1.7(m, |
| | | 3H), 1.1-0.9(m, |
| | | 2H). |
| 65. | 4-{[(2-Chloro-pyridine-4-carbonyl)- | M.S. (M⁺+ 1) 388 |
| | amino]-methyl}-piperidine-1-carboxylic | |
| | acid benzyl ester | |
| 66. | 4-{[(6-Amino-pyridine-3-carbonyl)- | M.S. (M⁺+ 1) 369 |
| | amino]-methyl}-piperidine-1-carboxylic | |
| | acid benzyl ester | |
| 67. | 4-(Benzoylamino-methyl)-piperidine-1- | M.S. (M⁺+ 1) 353 |
| | carboxylic acid benzyl ester | |
| 68. | 4-[(3-Cyano-benzoylamino)-methyl]- | M.S. (M⁺+ 1) 378 |
| | piperidine-1-carboxylic acid benzyl ester | |
| 69. | 4-{[(Pyridine-4-carbonyl)-amino]- | M.S. (M⁺+ 1) 380 |
| | methyl}-piperidine-1-carboxylic acid | |
| | indan-2-yl ester | |
| 70. | 4-{[(2-Amino-pyridine-3-carbonyl)-amino]- | M.S. (M⁺+ 1) 369 |
| | methyl}-piperidine-1-carboxylic acid benzyl ester | |
| 71. | 4-[(4-Methylamino-benzoylamino)-methyl]- | M.S. (M⁺+ 1) 382 |
| | piperidine-1-carboxylic acid benzyl ester | |
| 72. | 4-[(4-Amino-benzoylamino)-methyl]- | M.S. (M⁺+ 1) 368 |
| | piperidine-1-carboxylic acid benzyl ester | |
| 73. | 4-[(4-Trifluoromethoxy-benzoylamino)-methyl]- | M.S. (M⁺+ 1) 437 |
| | piperidine-1-carboxylic acid benzyl ester | |
| 74. | 4-[(4-Fluoro-benzoylamino)-methyl]- | M.S. (M⁺+ 1) 371 |
| | piperidine-1-carboxylic acid benzyl ester | |
| 75. | 4-[(2-Amino-benzoylamino)-methyl]- | M.S. (M⁺+ 1) 368 |
| | piperidine-1-carboxylic acid benzyl ester | |
| 76. | 4-{[(5-Ethyl-2-methyl-2H-pyrazole-3- | M.S. (M⁺+ 1) 385 |
| | carbonyl)-amino]-methyl}-piperidine-1- | |
| | carboxylic acid benzyl ester | |
| 77. | 4-{[(6-Chloro-imidazo[1,2-a]pyridine-2- | M.S. (M⁺+ 1) 427 |
| | carbonyl)-amino]-methyl}-piperidine-1- | |
| | carboxylic acid benzyl ester | |
| 78. | 4-{[(4-Bromo-thiophene-3-carbonyl)-amino]- | M.S. (M⁺+ 1) 438 |
| | methyl}-piperidine-1-carboxylic acid benzyl ester | |
| 79. | 4-{[(Isoxazole-5-carbonyl)-amino]-methyl}- | M.S. (M⁺+ 1) 344 |
| | piperidine-1-carboxylic acid benzyl ester | |
| 80. | 4-{[(1H-Imidazole-2-carbonyl)-amino]-methyl}- | M.S. (M⁺+ 1) 343 |
| | piperidine-1-carboxylic acid benzyl ester | |
| 81. | 4-{[(3-Bromo-pyridine-4-carbonyl)-amino]- | M.S. (M⁺+ 1) 433 |
| | methyl}-piperidine-1-carboxylic acid benzyl ester | |
| 82. | 4-{[([1,6]Naphthyridine-2-carbonyl)-amino]- | M.S. (M⁺+ 1) 405 |
| | methyl}-piperidine-1-carboxylic acid benzyl ester | |
| 83. | 4-{[(1-Methyl-1H-imidazole-2-carbonyl)-amino]- | M.S. (M⁺+ 1) 357 |
| | methyl}-piperidine-1-carboxylic acid benzyl ester | |
| 84. | 4-{[(5-Bromo-pyridine-3-carbonyl)-amino]- | M.S. (M⁺+ 1) 432 |
| | methyl}-piperidine-1-carboxylic acid benzyl ester | |
| 85. | 4-{[(Isoxazole-3-carbonyl)-amino]-methyl}- | M.S. (M⁺+ 1) 344 |
| | piperidine-1-carboxylic acid benzyl ester | |
| 86. | 4-{[(6-Bromo-pyridine-3-carbonyl)-amino]- | M.S. (M⁺+ 1) 432 |
| | methyl}-piperidine-1-carboxylic acid benzyl ester | |
| 87. | 4-{[(2-Methyl-thiazole-4-carbonyl)-amino]- | M.S. (M⁺+ 1) 374 |
| | methyl}-piperidine-1-carboxylic acid benzyl ester | |
| 88. | 4-{[(Oxazole-5-carbonyl)-amino]-methyl}- | M.S. (M⁺+ 1) 344 |
| | piperidine-1-carboxylic acid benzyl ester | |
| 89. | 4-{[(Pyrimidine-2-carbonyl)-amino]-methyl}- | M.S. (M⁺+ 1) 355 |
| | piperidine-1-carboxylic acid benzyl ester | |
| 90. | 4-{[(1,4,5,6-Tetrahydro-cyclopentapyrazole- | M.S. (M⁺+ 1) 383 |
| | 3-carbonyl)-amino]-methyl}-piperidine- | |
| | 1-carboxylic acid benzyl ester | |
| 91. | 4-{[(2-Methylsulfanyl-thiazole-4-carbonyl)-amino]- | M.S. (M⁺+ 1) 406 |
| | methyl}-piperidine-1-carboxylic acid benzyl ester | |
| 92. | 4-{[(5-Methyl-thiazole-4-carbonyl)-amino]- | M.S. (M⁺+ 1) 374 |
| | methyl}-piperidine-1-carboxylic acid benzyl ester | |
| 93. | 4-{[(5-Methyl-2H-[1,2,4]triazole-3- | M.S. (M⁺+ 1) 358 |
| | carbonyl)-amino]-methyl}-piperidine-1- | |
| | carboxylic acid benzyl ester | |
| 94. | 4-{[(4-Phenyl-thiazole-2-carbonyl)-amino]- | M.S. (M⁺+ 1) 436 |
| | methyl}-piperidine-1-carboxylic acid benzyl ester | |
| 95. | 4-{[(5-Hydroxymethyl-3H-imidazole-4- | M.S. (M⁺+ 1) 373 |
| | carbonyl)-amino]-methyl}-piperidine-1- | |
| | carboxylic acid benzyl ester | |
| 96. | 4-{[(2-Methyl-thiazole-5-carbonyl)-amino]- | M.S. (M⁺+ 1) 374 |
| | methyl}-piperidine-1-carboxylic acid benzyl ester | |
| 97. | 4-{[(2-Methyl-1H-pyrrole-3-carbonyl)-amino]- | M.S. (M⁺+ 1) 356 |
| | methyl}-piperidine-1-carboxylic acid benzyl ester | |
| 98. | 4-{[(2-Methyl-thiophene-3-carbonyl)-amino]- | M.S. (M⁺+ 1) 373 |
| | methyl}-piperidine-1-carboxylic acid benzyl ester | |
| 99. | 4-{[(Thiophene-3-carbonyl)-amino]-methyl}- | M.S. (M⁺+ 1) 377 |
| | piperidine-1-carboxylic acid 4-fluoro-benzyl ester | |
| 100. | 4-{[(Thiophene-3-carbonyl)-amino]-methyl}- | M.S. (M⁺+ 1) 393 |
| | piperidine-1-carboxylic acid 4-chloro-benzyl ester | |
| 101. | 4-{[(Thiophene-3-carbonyl)-amino]-methyl}- | M.S. (M⁺+ 1) 385 |
| | piperidine-1-carboxylic acid indan-2-yl ester | |
| 102. | 4-{[(2H-Pyrazole-3-carbonyl)-amino]-methyl}- | M.S. (M⁺+ 1) 369 |
| | piperidine-1-carboxylic acid indan-2-yl ester | |
| 103. | 4-{[(1H-Imidazole-4-carbonyl)-amino]-methyl}- | M.S. (M⁺+ 1) 357 |
| | piperidine-1-carboxylic acid 4-methyl-benzyl ester | |
| 104. | 4-{[(1-Methyl-1H-pyrrole-2-carbonyl)- | M.S. (M⁺+ 1) 370 |
| | amino]-methyl}-piperidine-1-carboxylic | |
| | acid 4-methyl-benzyl ester | |
| 105. | 4-{[(1H-Imidazole-4-carbonyl)-amino]-methyl}- | M.S. (M⁺+ 1) 361 |
| | piperidine-1-carboxylic acid 4-fluoro-benzyl ester | |
| 106. | 4-{[(1H-Imidazole-4-carbonyl)-amino]-methyl}- | M.S. (M⁺+ 1) 369 |
| | piperidine-1-carboxylic acid indan-2-yl ester | |
| 107. | 4-{[(1-Methyl-1H-pyrrole-2-carbonyl)- | M.S. (M⁺+ 1) 390 |
| | amino]-methyl}-piperidine-1-carboxylic | |
| | acid 4-chloro-benzyl ester | |
| 108. | 4-{[(1-Methyl-1H-pyrroIe-2-carbonyl)- | M.S. (M⁺+ 1) 382 |
| | amino]-methyl}-piperidine-1-carboxylic | |
| | acid indan-2-yl ester | |
| 109. | 4-{[(1H-Pyrrole-2-carbonyl)-amino]-methyl}- | M.S. (M⁺+ 1) 360 |
| | piperidine-1-carboxylic acid 4-fluoro-benzyl ester | |
| 110. | 4-{[(1H-Pyrrole-2-carbonyl)-amino]-methyl}- | M.S. (M⁺+ 1) 368 |
| | piperidine-1-carboxylic acid indan-2-yl ester | |
| 111. | 4-{[(1H-Pyrazole-4-carbonyl)-amino]- | M.S. (M⁺+ 1) 427 |
| | methyl}-piperidine-1-carboxylic acid 4- | |
| | bromo-thiophen-3-ylmethyl ester | |
| 112. | 4-{[(Pyrazine-2-carbonyl)-amino]-methyl}- | M.S. (M⁺+ 1) 355 |
| | piperidine-1-carboxylic acid benzyl ester | |
| 113. | 4-{[(Quinoline-4-carbonyl)-amino]-methyl}- | M.S. (M⁺+ 1) 404 |
| | piperidine-1-carboxylic acid benzyl ester | |
| 114. | 4-{[(2,6-Dihydroxy-pyridine-4-carbonyl)-amino]- | M.S. (M⁺+ 1) 386 |
| | methyl}-piperidine-1-carboxylic acid benzyl ester | |
| 115. | 4-{[(1-Oxy-pyridine-4-carbonyl)-amino]-methyl}- | M.S. (M⁺+ 1) 370 |
| | piperidine-1-carboxylic acid benzyl ester | |
| 116. | 4-{[(Pyrimidine-4-carbonyl)-amino]-methyl}- | M.S. (M⁺+ 1) 355 |
| | piperidine-1-carboxylic acid benzyl ester | |
| 117. | 4-{[(1-Methyl-1H-pyrazole-3-carbonyl)-amino]- | M.S. (M⁺+ 1) 357 |
| | methyl}-piperidine-1-carboxylic acid benzyl ester | |
| 118. | 4-{[ (2-Methyl-2H-pyrazole-3-carbonyl)-amino]- | M.S. (M⁺+ 1) 357 |
| | methyl}-piperidine-1-carboxylic acid benzyl ester | |
| 119. | 4-{[(1-Methyl-1H-pyrazole-4-carbonyl)-amino]- | M.S. (M⁺+ 1) 357 |
| | methyl}-piperidine-1-carboxylic acid benzyl ester | |
| 120. | 4-{[([1,2,5]Thiadiazole-3-carbonyl)-amino]- | M.S. (M⁺+ 1) 361 |
| | methyl}-piperidine-1-carboxylic acid benzyl ester | |
| 121. | 4-{[(5-Bromo-pyridine-2-carbonyl)-amino]- | M.S. (M⁺+ 1) 432 |
| | methyl}-piperidine-1-carboxylic acid benzyl ester | |
| 122. | 4-{[(Pyrimidine-5-carbonyl)-amino]-methyl}- | M.S. (M⁺+ 1) 355 |
| | piperidine-1-carboxylic acid benzyl ester | |
| 123. | 4-{[(Pyrazolo[1,5-a]pyrimidine-3- | M.S. (M⁺+ 1) 394 |
| | carbonyl)-amino]-methyl}-piperidine-1- | |
| | carboxylic acid benzyl ester | |
| 124. | 4-{[(6-Bromo-pyridine-2-carbonyl)-amino]- | M.S. (M⁺+ 1) 432 |
| | methyl}-piperidine-1-carboxylic acid benzyl ester | |
| 125. | 4-{[(Benzothiazole-2-carbonyl)-amino]-methyl}- | M.S. (M⁺+ 1) 410 |
| | piperidine-1-carboxylic acid benzyl ester | |
| 126. | 4-{[(3,5-Dimethyl-1H-pyrrole-2-carbonyl)-amino]- | M.S. (M⁺+ 1) 370 |
| | methyl}-piperidine-1-carboxylic acid benzyl ester | |
| 127. | 4-{[(3-Methyl-pyridine-4-carbonyl)-amino]- | M.S. (M⁺+ 1) 368 |
| | methyl}-piperidine-1-carboxylic acid benzyl ester | |
| 128. | 4-{[(6-Cyano-pyridine-3-carbonyl)-amino]- | M.S. (M⁺+ 1) 379 |
| | methyl}-piperidine-1-carboxylic acid benzyl ester | |
| 129. | 4-{[(2-Methyl-pyridine-4-carbonyl)-amino]- | M.S. (M⁺+ 1) 368 |
| | methyl}-piperidine-1-carboxylic acid benzyl ester | |
| 130. | 4-{[(2-Methoxy-6-methyl-pyridine-4-carbonyl)-amino]- | M.S. (M⁺+ 1) 398 |
| | methyl}-piperidine-1-carboxylic acid benzyl ester | |
| 131. | 4-{[(2-Chloro-6-methyl-pyridine-4-carbonyl)-amino]- | M.S. (M⁺+ 1) 402 |
| | methyl}-piperidine-1-carboxylic acid benzyl ester | |
| 132. | 4-{[(6-Amino-pyridazine-3-carbonyl)-amino]- | M.S. (M⁺+ 1) 370 |
| | methyl}-piperidine-1-carboxylic acid benzyl ester | |
| 133. | 4-[(2-Hydroxy-benzoylamino)-methyl]- | M.S. (M⁺+ 1) 369 |
| | piperidine-1-carboxylic acid benzyl ester | |
| 134. | 4-[(3-Hydroxy-benzoylamino)-methyl]- | M.S. (M⁺+ 1) 369 |
| | piperidine-1-carboxylic acid benzyl ester | |
| 135. | 4-[(2,5-Dihydroxy-benzoylamino)-methyl]- | M.S. (M⁺+ 1) 385 |
| | piperidine-1-carboxylic acid benzyl ester | |
| 136. | 4-[(4-Hydroxy-3,5-diiodo-benzoylamino)-methyl]- | M.S. (M⁺+ 1) 621 |
| | piperidine-1-carboxylic acid benzyl ester | |

### Example 137

1H-Pyrazole-4-carboxylic acid [1-(3-phenyl-propionyl)-piperidin-4-ylmethyl]-amide

Step 1:

1H-Pyrazole-4-carboxylic acid (piperidin-4-ylmethyl)-amide

4-{[(1H-Pyrazole-4-carbonyl)-amino]-methyl}-piperidine-1-carboxylic acid benzyl ester (EXAMPLE 34) (600 mg, 1.75 mmol), 10% palladium on Carbon (150 mg) and ethanol (15 mL) were combined in a Parr® jar and hydrogenated at 50 psi for 24 h. The reaction mixture was filtered through Celite® and the filtrate was evaporated in vacuo to give the product as a white foam.

Step 2

1H-Pyrazole-4-carboxylic acid [1-(3-phenyl-propionyl)-piperidin-4-ylmethyl]-amide

1H-Pyrazole-4-carboxylic acid (piperidin-4-ylmethyl)-amide (352 mg, 1.69 mmol), hydrocinnamoyl chloride (503 μL, 3.38 mmol), diisopropylethylamine (294 μL, 1.69 mmol) and DMF (4 mL) were combined under Nitrogen and stirred at 25° C. for 24 h. Sodium hydroxide (1 mL, 2N) was added and the mixture was stirred 1 h. Water was added and the contents of the reaction flask were extracted with EtOAc (3×50 mL). The combined organic extracts were dried with Na₂SO₄ and filtered. The filtrate was removed in vacuo and the remaining residue was purified using an ISCO® normal phase silica chromatography system (CH₂Cl₂ (100%) to CH₂Cl₂:MeOH:NH₄OH 90:10:1). Fractions containing the desired product were combined and the solvent was removed in vacuo to give a colorless oil. Addition of EtOAc followed by 1N HCl/Et₂O gave the product as a white solid.

¹H NMR (500 MHz, δ, DMSO-d₆): 8.10 (m, 1H), 8.04 (s, 2H), 7.28-7.20 (m, 4H), 7.18-7.14 (m, 1H), 4.38 (m, 1H), 3.85 (m, 1H), 3.06 (m, 2H), 2.90 (m, 1H), 2.80 (t, 2H), 2,60 (m, 2H), 1.75-1.60 (m, 4H), 0.95 (m, 2H).

The following compounds were prepared by substituting the appropriate acid chloride for the hydrocinnamoyl chloride in the above procedure.

| EX. | Name | Analytical Data |
|---|---|---|
| 138 | 1H-Pyrazole-4-carboxylic acid[1- | ¹H NMR(500 MHz, δ, DMSO-d₆): 8.08- |
| | (2-phenyl-cyclopropanecarbonyl)- | 7.98(m, 3H), 7.26(m, 2H), 7.17(m, 3H), 4.38(m, |
| | piperidin-4-ylmethyl]-amide | 1H), 4.16(m, 1H), 3.15-2.97(m, 3H), |
| | | 2.58(m, 1H), 2.26(m, 2H), 1.80-1.60(m, |
| | | 3H), 1.30(m, 1H), 1.20-0.95(m, 3H). |
| 139 | 1H-Pyrazole-4-carboxylic acid[1- | ¹H NMR(500 MHz, δ, DMSO-d₆): 8.16(s, br, 1H), |
| | (3-phenyl-acryloyl)-piperidin-4- | 8.07(m, 1H), 7.88(s, br, 1H), 7.70(m, 2H), |
| | ylmethyl]-amide | 7.48-7.34(m, 4H), 7.26(m, 2H), 4.48(m, |
| | | 1H), 4.29(m, 1H), 3.17-3.00(m, |
| | | 3H), 2.65(m, 1H), 1.85-1.69(m, |
| | | 3H), 1.15-1.00(m, 2H). |

The following examples were prepared from 1H-pyrazole-4-carboxylic acid (piperidin-4-ylmethyl)-amide as described in Example 1 Step 2.

| EX. | Name | Analytical Data |
|---|---|---|
| 140 | [1-Benzyl-2-oxo-2-(4-{[(1H-pyrazole-4- | M.S. (M⁺ + 1) 456 |
| | carbonyl)-amino]-methyl}-piperidin-1- | |
| | yl)-ethyl]-carbamic acid tert-butyl ester | |
| 141 | [1-(4-Chloro-benzyl)-2-oxo-2-(4-{[(1H- | M.S. (M⁺ + 1) 490 |
| | pyrazole-4-carbonyl)-amino]-methyl}- | |
| | piperidin-1-yl)-ethyl]-carbamic acid | |
| | tert-butyl ester | |
| 142 | 1H-Pyrazole-4-carboxylic acid[1-(2- | M.S. (M⁺ + 1) 357 |
| | hydroxy-3-phenyl-propionyl)-piperidin- | |
| | 4-ylmethyl]-amide | |
| 143 | 1H-Pyrazole-4-carboxylic acid[1-(2- | M.S. (M⁺ + 1) 355 |
| | methyl-3-phenyl-propionyl)-piperidin-4- | |
| | ylmethyl]-amide | |
| 144 | 1H-Pyrazole-4-carboxylic acid{1-[2- | M.S. (M⁺ + 1) 373 |
| | hydroxy-3-(4-hydroxy-phenyl)- | |
| | propionyl]-piperidin-4-ylmethyl}-amide | |
| 145 | 1H-Pyrazole-4-carboxylic acid[1-(2- | M.S. (M⁺ + 1) 353 |
| | phenyl-cyclopropanecarbonyl)- | |
| | piperidin-4-ylmethyl]-amide | |

The following two compounds were prepared from EXAMPLES 140 and 141 respectively by treatment with trifluoroacetic acid in dichloromethane.

| EX. | Name | Analytical Data |
|---|---|---|
| 146 | 1H-Pyrazole-4-carboxylic | M.S. (M⁺ + 1) 356 |
| | acid[1-(2-amino-3- | |
| | phenyl-propionyl)-piperidin- | |
| | 4-ylmethyl]-amide | |
| 147 | 1H-Pyrazole-4-carboxylic | M.S. (M⁺ + 1) 390 |
| | acid{1-[2-amino-3- | |
| | (4-chloro-phenyl)-propionyl]- | |
| | piperidin-4-ylmethyl}-amide | |

### Example 148

Trans 1H-Pyrazole-4-carboxylic acid [1-(2-phenyl-cyclopropylmethyl)-piperidin-4-ylmethyl]-amide

A solution of 1H-pyrazole-4-carboxylic acid (piperidin-4-ylmethyl)-amide (290 mg, 1.39 mmol), trans-2-phenylcyclopropanecarbaldehyde (224 mg, 1.53 mmol) and sodium triacetoxyborohydride (590 mg, 2.78 mmol) in MeOH (15 mL) was heated to 50° C. and stirred for 1 h. The resulting reaction mixture was concentrated and purified by silica gel chromatography (gradient: CH₂Cl₂ to 80:20:2 CH₂Cl₂:MeOH:NH4 OH) to give the trans 1H-pyrazole-4-carboxylic acid [1-(2-phenyl-cyclopropylmethyl)-piperidin-4-ylmethyl]-amide product.

¹H NMR (δ, CDCl₃): 7.86 (s, 2H), 7.23 (d, 2H), 7.17 (t, 1H), 7.02 (d, 2H), 5.94 (brt, 1H), 3.35 (m, 2H), 3.10 (brt, 2H), 2.55 (dd, 1H), 2.39 (dd, 1H), 2.03 (q, 2H), 1.70-1.55 (m, 4H), 1.41 (m, 2H), 1.22 (m, 1H), 0.95 (m, 1H), 0.82 (m, 1H).

The following compounds were prepared similarly to the procedure described above for EXAMPLE 148 but substituting the appropriate aldehyde for the trans-2-phenylcyclopropanecarbaldehyde.

| EX. | Name | Analytical Data |
|---|---|---|
| 149 | 1H-Pyrazole-4-carboxylic | ¹H NMR(δ, CDCl₃): 7.93(s, 2H), 7.3-7.15(m, |
| | acid[1-(3-phenyl-propyl)- | 5H), 6.30(brt, 1H), 3.35(t, 2H), 3.04(brd, |
| | piperidin-4-ylmethyl]-amide | 2H), 2.61(t, 2H), 2.46(dd, 2H), 2.04(t, |
| | | 2H), 1.88(m, 2H), 1.70(m, 2H), |
| | | 1.47(m, 2 H), 1.27(t, 1H). |
| 150 | 1H-Pyrazole-4-carboxylic | ¹H NMR(δ, CD₃OD): 8.03(s, 2H), 7.3-7.1(m, |
| | acid[1-(4-phenyl-butyl)- | 5H), 3.21(d, 2H), 2.97(brd, 2H), 2.63(t, |
| | piperidin-4-ylmethyl]-amide | 2H), 2.40(dd, 2H), 2.01(t, 2H), 1.76(brd, |
| | | 2H), 1.7-1.5(m, 5H), 1.30(m, 2H). |
| 151 | 1H-Pyrazole-4-carboxylic | M.S. (M⁺ + 1) 313 |
| | acid(1-phenethyl-piperidin-4- | |
| | ylmethyl)-amide | |
| 152 | 1H-Pyrazole-4-carboxylic acid[1- | M.S. (M⁺ + 1) 339 |
| | (2-phenyl-cyclopropylmethyl)- | |
| | piperidin-4-ylmethyl]-amide | |
| 153 | 1H-Pyrazole-4-carboxylic acid[1- | ¹H NMR(δ, CDCl₃): 7.86(s, 2H), 7.23(d, 2H), |
| | (2-phenyl-cyclopropylmethyl)- | 7.17(t, 1H), 7.00(d, 2H), 6.61(brs, 1H), 3.30(m, |
| | piperidin-4-ylmethyl]-amide | 2H), 3.10(brt, 2H), 2.55(dd, 1H), 2.39(dd, |
| | | 1H), 2.03(q, 2H), 1.70-1.55(m, 4H), 1.41(m, |
| | | 2H), 1.22(m, 1H), 0.95(m, 1H), 0.82(m, 1H). |

The following compounds were prepared as described above for EXAMPLE 148, but replacing 1H-pyrazole-4-carboxylic acid (piperidin-4-ylmethyl)-amide with, for example, 4-hydroxy-N-piperidin-4-ylmethyl-benzamide, which was prepared from 4-[(4-hydroxy-benzoylamino)-methyl]-piperidine-1-carboxylic acid benzyl ester as described in EXAMPLE 137, step 1.

| EX. | Name | Analytical Data |
|---|---|---|
| 154 | 4-Hydroxy-N-[1-(2-phenyl- | ¹H NMR(δ, CDCl₃): 7.43(d, 2H), 7.3-7.1(m, 3H), |
| | cyclopropylmethyl)-piperidin- | 7.00(d, 2H), 6.65 d, 2H), 6.39(brt, 1H), 3.35(m, |
| | 4-ylmethyl]-benzamide | 2H), 3.14(brt, 2H), 2.58(dd, 1H), 2.41(dd, 1H), |
| | | 2.08(q, 2H), 1.7-1.5(m, 4H), 1.41(m, 2H), |
| | | 1.22(m, 1H), 0.96(m, 1H), 0.82(m, 1H). |
| 155 | 4-Hydroxy-N-[1-(3-phenyl-propyl)- | ¹H NMR(δ, CD₃OD): 7.70(d, 2H), 7.3-7.1(m, 5H), |
| | piperidin-4-ylmethyl]-benzamide | 6.80(d, 2H), 3.23(d, 2H), 3.02(brd, 2H), 2.61(dd, |
| | | 2H), 2.42(dd, 2H), 2.08(brt, 2H), 1.9-1.6(m, 5H), |
| | | 1.35(m, 2H). |

### Example 156

4-{[(Pyridine-4-carbonyl)-amino]-methyl}-piperidine-1-carboxylic acid 4-cyclopropyl-benzyl ester

Step 1:

4-Cyclopropyl-benzoic acid ethyl ester

Indium trichloride (2.2 g, 10 mmol) and THF (50 mL) were combined under nitrogen and cooled to −70° C. Cyclopropylmagnesium bromide solution (33 mL, 30 mmol, 0.92M) was added dropwise while maintaining the reaction temperature≦−60° C. After the addition was complete the reaction was stirred 0.5 h with cooling then 0.5 h with the cooling bath removed. The resulting solution was added via cannula to a refluxing solution of ethyl-4-iodobenzoate (5.5 g, 20 mmol), trans-dichlorobis(triphenylphosphine)palladium(II) (421 mg, 0.60 mmol) and THF (100 mL) under nitrogen. After 24 h, the contents of the reaction flask were cooled and the solvent was removed in vacuo. Water (100 mL) and 5% KHSO₄ were added and the mixture was extracted with CH₂Cl₂ (3×100 mL). The combined organic extracts were washed with brine, dried with Na₂SO₄ and filtered. The filtrate was removed in vacuo and the remaining residue was purified by flash column chromatography (hexane:EtOAc 95:5) to give the 4-cyclopropyl-benzoic acid ethyl ester as an orange oil.

Step 2

(4-Cyclopropyl-phenyl)-methanol

4-Cyclopropyl-benzoic acid ethyl ester (2.46 g, 13 mmol), and THF (250 mL) were combined under nitrogen and cooled in an IPA/dry ice bath to −70° C. Lithium aluminum hydride solution (20 mL, 20 mmol, 11.0M) was added dropwise. After 2 h excess lithium aluminum hydride was quenched by adding EtOAc dropwise. The reaction was warmed to 25° C. then the solvent was removed in vacuo. Water (200 mL) and a few drops of HCl(aq, 6N) were added. The mixture was extracted with EtOAc (3×100 mL). The combined organic extracts were washed with brine, dried with Na₂SO₄ and filtered. The filtrate was removed in vacuo and the remaining residue was purified by flash column chromatography (hexane:EtOAc 40:60) to give the (4-cyclopropyl-phenyl)-methanol as a colorless oil.

Step 3

Carbonic acid 4-cyclopropyl-benzyl ester 2,5-dioxo-pyrrolidin-1-yl ester

The title compound was prepared from (4 Cyclopropyl-phenyl)-methanol as described above for similar compounds (Chem. Pharm. Bull., 38(1):110-115(1990)).

Step 4

4-Aminomethyl-piperidine-1-carboxylic acid 4-cyclopropyl-benzyl ester

The title compound was prepared from carbonic acid 4-cyclopropyl-benzyl ester 2,5-dioxo-pyrrolidin-1-yl ester as described in EXAMPLE 1, Step 1

Step 5

4-{[(Pyridine-4-carbonyl)-amino]-methyl}-piperidine-1-carboxylic acid 4-cyclopropyl-benzyl ester

The title compound was prepared from 4-aminomethyl-piperidine-1-carboxylic acid 4-cyclopropyl-benzyl ester as described above in EXAMPLE 1, Step 2.

M.S. (M⁺+1) 394

The following compounds were prepared from 4-aminomethyl-piperidine-1-carboxylic acid 4-cyclopropyl-benzyl ester as described above in EXAMPLE 1, step 2.

| EX. | Name | Analytical Data |
|---|---|---|
| 157 | 4-[(4-Hydroxy-benzoylamino)-methyl]- | M.S. (M⁺ + 1) 409 |
| | piperidine-1-carboxylic acid 4- | |
| | cyclopropyl-benzyl ester | |
| 158 | 4-{[(1H-Pyrazole-3-carbonyl)-amino]- | M.S. (M⁺ + 1) 383 |
| | methyl}-piperidine-1-carboxylic acid 4- | |
| | cyclopropyl-benzyl ester | |
| 159 | 4-{[(1H-Pyrazole-4-carbonyl)-amino]- | ¹H NMR(500 MHz δ, CDCl₃): 10.70(s, br, 1H), 7.95(s, |
| | methyl}-piperidine-1-carboxylic acid 4- | 2H), 7.25(d, 2H), 7.05(d, 2H), 6.00(m, 1H), 5.06(s, |
| | cyclopropyl-benzyl ester | 2H), 4.20(s, br, 2H), 3,30(s, br, 2H),. 2.75(s, br, |
| | | 2H), 1.90(m, 1H), 1.85-1.50(m, 3H), 1.20(m, 2H), |
| | | 0.97(m, 2H), 0.68(m, 2H). |

The following compounds were prepared from 4-hydroxy-N-piperidin-4-ylmethyl-benzamide (prepared from 4-[(4-hydroxy-benzoylamino)-methyl]-piperidine-1-carboxylic acid benzyl ester as described in EXAMPLE 137, step 1) as described in EXAMPLE 1, Step 2.

| EX. | Name | Analytical Data |
|---|---|---|
| 160 | 4-Hydroxy-N-[1-(2-phenyl- | ¹H NMR(δ, CDCl₃): 8.72(brs, 1H), 7.61(d, 2H), 7.24(m, |
| | cyclopropanecarbonyl)- | 2H), 7.19(t, 1H), 7.06(d, 2H), 6.93(d, 2H), 6.72(brs, |
| | piperidin-4-ylmethyl]-benzamide | 1H), 4.55(brd, 1H), 4.10(brd, 1H), 3.3-3.1(m, |
| | | 2H), 3.01(q, 1H), 2.58(brt, 1H), 2.41(brs, 1H), |
| | | 2.0-1.6(m, 5H), 1.3-1.1(m, 3H). |
| 161 | 4-Hydroxy-N-[1-(2-phenyl- | M.S. (M⁺ + 1) 379 |
| | cyclopropanecarbonyl)-piperidin- | |
| | 4-ylmethyl]-benzamide | |

### Example 162

1H-Pyrazole-4-carboxylic acid (1-benzylthiocarbamoyl-piperidin-4-ylmethyl)-amide

1H-Pyrazole-4-carboxylic acid (piperidin-4-ylmethyl)-amide (EXAMPLE 137, Step 1) (50 mg, 0.24 mmol), benzyl isothiocyanate (35 μL,

0.264 mmol) and DMF (1 mL) were combined and stirred under Nitrogen for 1 h. The contents of the reaction flask were poured into water and sodium hydroxide (2 mL, 2N) was added. The resulting mixture was extracted with EtOAc (3×50 mL) and the combined organic extracts were dried with Na₂SO₄. The filtrate was removed in vacuo and the remaining residue was purified by Gilson® reverse phase preparative HPLC. The fraction containing the desired product was evaporated in vacuo to give a colorless oil. Trituration with EtOAc/EtOH afforded the EXAMPLE 162 as a white solid.

¹H NMR (500 MHz, 6, DMSO-d₆): 13.10 (s, 1H), 8.20 (m, 2H), 8.10 (m, 1H), 7.90 (m, 1H), 7.32-7.18 (m, 5H), 4.80 (d, 2H), 4.65 (d, 2H), 3.10 (t, 2H), 2.97 (t, 2H), 1.80 (m, 1H), 1.67 (m, 2H), 1.10 (m, 2H).

### Example 163

4-{[(1H-Pyrazole-4-carbonyl)-amino]-methyl}-piperidine-1-carboxylic acid benzylamide

The title compound was prepared as described in EXAMPLE 162 except that benzyl isocyanate was used instead of benzyl isothiocyanate.

¹H NMR (500 MHz, 6, DMSO-d₆): 13.10 (s, 1H), 8.16 (s, 1H), 8.04 (m, 1H), 7.88 (s, 1H), 7.30-7.16 (m, 4H), 7.02 (m, 1H), 4.21 (d, 2H), 3.99 (d, 2H), 3.10 (t, 2H), 2.65 (m, 2H), 1.72-1.58 (m, 3H), 1.05-0.95 (m, 2H).

### Example 164

1H-Pyrazole-4-carboxylic acid [1-(2-hydroxy-3-phenyl-propyl)-piperidin-4-ylmethyl]-amide

To a solution of 2-benzyloxirane (0.0 mL, 0.07 mmol) in iso-propyl alcohol (5 mL) was added 1H-pyrazole-4-carboxylic acid (piperidin-4-ylmethyl)-amide (EXAMPLE 137, Step 1) (15 mg, 0.07 mmol). The resulting reaction mixture was heated to 60° C. for 24 h. The reaction mixture was concentrated, partitioned between EtOAc and aqueous sodium bicarbonate. The organic phase was dried, the solvent evaporated, and the crude product purified by reverse phase HPLC to give 1H-Pyrazole-4-carboxylic acid [1-(2-hydroxy-3-phenyl-propyl)-piperidin-4-ylmethyl]-amide.

M.S. (M⁺+1) 343

### Example 165

4-{[(2-Oxo-1,2-dihydro-pyridine-4-carbonyl)-amino]-methyl}-piperidine-1-carboxylic acid benzyl ester

To 4-{[(1-oxy-pyridine-4-carbonyl)-amino]-methyl}-piperidine-1-carboxylic acid benzyl ester (EXAMPLE 115) (200 mg, 0.542 mmol) was added acetic anhydride (5 mL) and the mixture heated to reflux for 24 h. The reaction was concentrated and chromatographed on silica using ethyl acetate to give an oil (40 mg). The crude material was dissolved in methanol (10 mL) and treated with solid potassium carbonate (40 mg) for 0.5 h. Concentration of the reaction and extraction into dichloromethane (20 mL) from aqueous sodium bicarbonate (20 mL) followed by concentration and precipitation of the solid from ether gave the 4-{[(2-Oxo-1,2-dihydro-pyridine-4-carbonyl)-amino]-methyl}-piperidine-1-carboxylic acid benzyl ester.

M.S.(M+1): 370

### Example 166

4-{[(2-Methylaminomethyl-pyridine-4-carbonyl)-amino]-methyl}-piperidine-1-carboxylic acid benzyl ester

Step 1:

Preparation of 2,4-pyridinedicarboxcyclic acid diethyl ester

To a mixture of 2,4-pyridinedicarboxylic acid (23 g, 0.138 mol) in ethanol (500 mL) was bubbled anhydrous hydrogen chloride gas over a period of 6 h. The resulting reaction mixture was concentrated in vacuo and extracted into dichloromethane (500 mL) from 10% aqueous sodium bicarbonate (500 mL). The organic extract was dried over sodium sulfate, and concentrated in vacuo to give 2,4-pyridinedicarboxcyclic acid diethyl ester as an oil.

M.S.(M+1): 224

Step 2:

Preparation of 2-Formyl-isonicotinic acid ethyl ester

To a solution of 2,4-pyridinedicarboxcyclic acid diethyl ester (25 g, 0.112 mol) in tetrahydrofuran (1 L) at −78° C. and under nitrogen was slowly added a solution of 1.0 M diisobutylaluminum hydride in THF (11 mL). The reaction was stirred at −78° C. for 5 h and then quenched by addition of a solution of tetrahydrofuran-acetic acid-water (174 mL, 62 mL, 15 mL) and the reaction allowed to warm to room temperature. Diethyl ether (500 mL) and 10% aqueous sodium bicarbonate (1 L) were added and the mixture stirred for 0.5 h. The ether layer was removed and the aqueous layer extracted with ethyl acetate (4×500 mL) The combined organic extracts were washed with saturated sodium chloride and concentrated to an oil which was purified by silica gel column chromatography using 30% ethyl acetate/hexane as eluent to give 2-formyl-isonicotinic acid ethyl ester as an oil.

M.S.(M+1): 180

Step 3:

Preparation of 2-Diethoxymethyl-isonicotinic acid ethyl ester

To a solution of 2-formyl-isonicotinic acid ethyl ester (5.0 g, 0.027 mol) in ethanol (9 mL) was added triethyl orthoformate (6.2 mL, 0.037 mol) followed by a solution of 6N hydrochloric acid in ethanol (1.5 mL). The mixture was heated to 110° C. (reflux) for 1.5 h, cooled to rt and solid potassium carbonate (1.80 g) added. The mixture was stirred for 5 min, concentrated in vacuo, and redissolved in diethyl ether (100 mL). The reaction was filtered through silica and the resulting cake washed with diethyl ether (50 mL). The filtrated was concentrated in vacuo to give 2-diethoxymethyl-isonicotinic acid ethyl ester as an oil.

M.S.(M+1): 254

Step 4:

Preparation of 2-Diethoxymethyl-isonicotinic acid

To a solution of 2-diethoxymethyl-isonicotinic acid ethyl ester (3.0 g, 0.012 mol) in tetrahydrofuran (100 mL) was added 1N sodium hydroxide (24 mL, 0.024 mol) and mixture allowed to stir for 2 h at rt. The reaction was concentrated in vacuo to give a pasty solid of 2-diethoxymethyl-isonicotinic acid, which was used in the next step as is.

M.S.(M+1): 226

Step 5:

Preparation of 4-{[(2-Diethoxymethyl-pyridine-4-carbonyl)-amino]-methyl}-piperidine-1-carboxylic acid benzyl ester

4-{[(2-Diethoxymethyl-pyridine-4-carbonyl)-amino]-methyl}-piperidine-1-carboxylic acid benzyl ester was prepared in a similar manner as described in EXAMPLE 1, Step 2.

M.S.(M+1): 456

Step 6:

Preparation of 4{[(2-Formyl-pyridine-4-carbonyl)-amino]-methyl}-piperidine-1-carboxylic acid benzyl ester

To a solution of 4-{[(2-diethoxymethyl-pyridine-4-carbonyl)-amino]-methyl}-piperidine-1-carboxylic acid benzyl ester (1.3 g, 0.0029 mol) in dioxane (20 mL) was added 1N hydrochloric acid (40 mL) and the mixture was warmed to 50° C. for 1.5 h. The reaction was cooled, diluted with ethyl acetate (100 mL) and 10% aqueous sodium bicarbonate (100 mL), and stirred well. The organic layer was removed, dried over sodium sulfate, filtered and concentrated in vacuo to give 4-{[(2-formyl-pyridine-4-carbonyl)-amino]-methyl}-piperidine-1-carboxylic acid benzyl ester as an oil.

M.S.(M+1): 382

Step 7:

Prep of 4-{[(2-Methylaminomethyl-pyridine-4-carbonyl)-amino]-methyl}-piperidine-1-carboxylic acid benzyl ester

To a solution of 4{[(2-formyl-pyridine-4-carbonyl)-amino]-methyl}-piperidine-1-carboxylic acid benzyl ester (50 mg, 0.13 mmol) in dichloroethane (0.5 mL) was added acetic acid (8 μL, 0.13 mmol), 2.0M methylamine in THF (72 μL, 0.14 mmol) followed by sodium triacetoxyborohydride (42 mg, 0.20 mmol). The resulting mixture was stirred for 5 h. The reaction was concentrated in vacuo and the residue chromatographed (reverse phase C-18 using acetonitrile/0.1% trifluoroacetic acid in water) to give upon concentration in vacuo 4{[(2-methylaminomethyl-pyridine-4-carbonyl)-amino]-methyl}-piperidine-1-carboxylic acid benzyl ester as the trifluoroacetic acid salt.

M.S. (M⁺+1) 397

The following compounds were prepared as described above for 4-{[(2-methylaminomethyl-pyridine-4-carbonyl)-amino]-methyl}-piperidine-1-carboxylic acid benzyl ester, replacing methylamine with the appropriate amine in step 7, EXAMPLE 166.

| EX. | Name | Analytical Data |
|---|---|---|
| 167 | 4-{[(2-Dimethylaminomethyl-pyridine- | M.S. (M⁺ + 1) 411 |
| | 4-carbonyl)-amino]-methyl}-piperidine- | |
| | 1-carboxylic acid benzyl ester | |
| 168 | 4-{[(2-Aminomethyl-pyridine-4- | M.S. (M⁺ + 1) 383 |
| | carbonyl)-amino]-methyl}-piperidine-1- | |
| | carboxylic acid benzyl ester | |

### Example 169

4-{[(2-Hydroxymethyl-pyridine-4-carbonyl)-amino]-methyl}-piperidine-1-carboxylic acid benzyl ester

To a solution of 4-{[(2-formyl-pyridine-4-carbonyl)-amino]-methyl}-piperidine-1-carboxylic acid benzyl ester (EXAMPLE 166, Step 6) (50 mg, 0.131 mmol) in ethanol (2 mL) was added sodium borohydride (5 mg) and the mixture stirred for 0.5 h. The reaction was diluted with 10% aqueous sodium bicarbonate (10 mL) and extracted with ethyl acetate (25 mL). The ethyl acetate extract was concentrated and chromatographed (reverse phase C-18 using acetonitrile/0.1% trifluoroacetic acid in water) to give upon concentration in vacuo the 4-{[(2-hydroxymethyl-pyridine-4-carbonyl)-amino]-methyl}-piperidine-1-carboxylic acid benzyl ester as the trifluoroacetic acid salt.

M.S.(M+1): 384

### Example 170

4-({[2-(1-Hydroxy-ethyl)-pyridine-4-carbonyl]-amino}-methyl)-piperidine-1-carboxylic acid benzyl ester

To a solution of 4-{[(2-formyl-pyridine-4-carbonyl)-amino]-methyl}-piperidine-1-carboxylic acid benzyl ester (EXAMPLE 166, Step 6) (50 mg, 0.131 mmol) in THF (2 mL) at −78° C. was added 3.0M methylmagnesium chloride (45 μL, 0.135 mmol). The mixture was stirred for 5 min and allowed to warm to rt. The reaction was diluted with 10% aqueous sodium bicarbonate (10 mL) and extracted with ethyl acetate (25 mL). The ethyl acetate extract was concentrated and chromatographed on silica using 100% ethyl acetate to ethyl acetate/methanol (95/5) to give the 4-({[2-(1-hydroxy-ethyl)-pyridine-4-carbonyl]-amino}-methyl)-piperidine-1-carboxylic acid benzyl ester.

M.S. (M⁺1) 398

### Example 171

4-({[2-(2,4-Dimethoxy-benzylamino)-pyridine-4-carbonyl]-amino}-methyl)-piperidine-1-carboxylic acid benzyl ester

A mixture of 4-{[(2-chloro-pyridine-4-carbonyl)-amino]-methyl}-piperidine-1-carboxylic acid benzyl ester (EXAMPLE 65) (310 mg, 0.8 mmol) and 2,4-dimethoxybenzylamine (1 mL) were heated to 140° C. for 18 h, cooled to rt, and partitioned between pH5.2 citrate buffer and EtOAc. The organic layer was dried and the solvent evaporated to give the crude product, purified by chromatography on silica (1:1 hexane EtOAc to 5% MeOH EtOAc to give the 4-({[2-(2,4-Dimethoxy-benzylamino)-pyridine-4-carbonyl]-amino}-methyl)-piperidine-1-carboxylic acid benzyl ester.

M.S. (M+1) 519

### Example 172

4-{[(2-Amino-pyridine-4-carbonyl)-amino]-methyl}-piperidine-1-carboxylic acid benzyl ester

4-({[2-(2,4-Dimethoxy-benzylamino)-pyridine-4-carbonyl]-amino}-methyl)-piperidine-1-carboxylic acid benzyl ester (EXAMPLE 171) (124 mg) in dichloromethane (5 mL) was treated with trifluoroacetic acid (0.5 mL). After 30 min, the reaction mixture was partitioned between EtOAc and dilute sodium bicarbonate solution. The organic layer was washed with brine, dried and the solvent evaporated to give the crude product which was stirred with ether (3 mL) and filtered to give the 4-{[(2-amino-pyridine-4-carbonyl)-amino]-methyl}-piperidine-1-carboxylic acid benzyl ester as a white solid.

M.S. (M⁺+1) 369

### Example 173

4-({[2-(2-Dimethylamino-ethylamino)-pyridine-4-carbonyl]-amino}-methyl)-piperidine-1-carboxylic acid benzyl ester

A mixture of 4-{[(2-chloro-pyridine-4-carbonyl)-amino]-methyl}-piperidine-1-carboxylic acid benzyl ester (EXAMPLE 65) (50 mg, 0.8 mmol) and N,N-dimethylethylenediamine (0.2 mL) were heated to 100 C. for 18 hours, cooled to room temperature. The reaction mixture was then purified by reverse phase HPLC to give the 4-({[2-(2-dimethylamino-ethylamino)-pyridine-4-carbonyl]-amino}-methyl)-piperidine-1-carboxylic acid benzyl ester as its trifluoroacetate salt.

M.S. (M⁺+1) 440

### Example 174

N-[1-(2-Phenyl-ethanesulfonyl)-piperidin-4-ylmethyl]-isonicotinamide

Step 1:

4-Aminomethyl-piperidine-1-carboxylic acid tert-butyl ester

To a mixture of 15 g of 4-aminomethylpiperidine in 250 mL of anhydrous tetrahydrofuran cooled to −78° C. was added dropwise over 45 min a solution of 24 g of di-tert-butyl di-carbonate in 100 mL of anhydrous tetrahydrofuran. After stirring for 1 h at −78° C., the mixture was allowed to warm to rt and stirred overnight. The mixture was concentrated to near dryness and diluted with 200 mL of 10% aqueous citric acid. The mixture was extracted with 3×100 mL of ether, then made basic with sodium hydroxide pellets and extracted with 3×200 mL of chloroform. The combined chloroform extracts were dried over magnesium sulfate and concentrated to dryness under reduced pressure. The resulting oil was homogeneous by TLC (development with 90:10 chloroform saturated with ammonia: methanol).

¹H NMR (400 MHz, CDCl₃): 4.1 (br s, 2H), 2.7 (br m, 2H), 2.6 (d, 2H), 1.7 (m, 3H), 1.42 (s, 9H), 1.1 (m, 2H).

Step 2:

4-(Benzyloxycarbonylamino-methyl)-piperidine-1-carboxylic acid tert-butyl ester

To a solution of 21 g of 4-aminomethyl-piperidine-1-carboxylic acid tert-butyl ester in 10 mL of ethyl acetate cooled to 0° C. was added 100 mL of saturated sodium carbonate and 17 g of benzyl chloroformate. The solution was stirred for 3 h, then separated. The organic layer was dried over magnesium sulfate and concentrated under reduced pressure. Drying under vacuum gave the product as an oil:

¹H NMR (400 MHz, CDCl₃): 7.35 (m, 5H), 5.3 (d, 1H), 5.1 (s, 2H), 4.1 (br s, 2H), 3.0 (br m, 2H), 2.6 (br m, 2H), 1.7 (m, 3H), 1.42 (s, 9H), 1.1 (m, 2H).

Step 3:

Piperidin-4-ylmethyl-carbamic acid benzyl ester

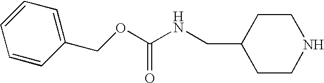

A mixture of 35 g of 4-(benzyloxycarbonylamino-methyl)-piperidine-1-carboxylic acid tert-butyl ester and 50 mL of 4N HCl in dioxane was stirred at rt for 3 h, then diluted with 200 mL of ether and filtered. The piperidin-4-ylmethyl-carbamic acid benzyl ester hydrochloride salt was obtained as a white fluffy solid. The free base was obtained by partitioning the hydrochloride between 50 mL chloroform and 50 mL saturated aqueous Na₂CO₃.

MS (m+1)=249;

¹H NMR (400 MHz, CDCl₃)): 7.35 (m, 5H), 5.15 (s, 2H), 4.9 (br s, 1H), 3.1 (m, 2H), 2.6 (m, 3H), 1.7 (m, 2H), 1.6 (m, 2H), 1.1 (m, 2H).

Step 4:

[1-(2-Phenyl-ethenesulfonyl)-piperidin-4-ylmethyl]-carbamic acid benzyl ester

A mixture of 2 g of piperidin-4-ylmethyl-carbamic acid benzyl ester hydrochloride, 25 mL of dichloromethane, 1.5 grams of trans-2-styrenesulfonyl chloride, and 3 mL of N,N-diisopropylethylamine was stirred at rt overnight, then diluted with 200 mL of chloroform, and washed with 100 mL of saturated sodium carbonate. The chloroform extracts were dried over magnesium sulfate and concentrated. The [1-(2-phenyl-ethenesulfonyl)-piperidin-4-ylmethyl]-carbamic acid benzyl ester was obtained as a white solid.

MS (m+1)=415;

¹H NMR (400 MHz, CDCl₃)): 7.5-7.2 (m, 10H), 6.65 (m, 1H), 5.15 (s, 2H), 4.8 (br s, 1H), 3.8 (d, 2H), 3.1 (dd, 2H), 2.6 (dd, 2H), 1.8 (d, 2H), 1.6 (m, 2H), 1.35 (m, 2H).

Step 5:

C-[1-(2-Phenyl-ethanesulfonyl)-piperidin-4-yl]-methylamine

A mixture of 2.5 g of [1-(2-phenyl-ethenesulfonyl)-piperidin-4-ylmethyl]-carbamic acid benzyl ester, 1 g of 20% palladium hydroxide on carbon, 200 mL of methanol and 50 mL of tetrahydrofuran were shaken under 50 psi of hydrogen for 2 days at rt. The catalyst was filtered off and washed with 250 mL of methanol. Concentration under reduced pressure gave the C-[1-(2-phenyl-ethanesulfonyl)-piperidin-4-yl]-methylamine as a white solid.

MS (m+1)=283;

¹H NMR (400 MHz, CDCl₃)): 7.4-7.2 (m, 5H), 5.1 (s, 2H), 3.8 (d, 2H), 3.1 (m, 4H), 2.7 (dd, 2H), 1.8 (d, 2H), 1.6 (m, 5H), 1.3 (m, 2H).

Step 6:

N-[1-(2-Phenyl-ethanesulfonyl)-piperidin-4-ylmethyl]-isonicotinamide

The N-[1-(2-Phenyl-ethanesulfonyl)-piperidin-4-ylmethyl]-isonicotinamide was prepared from C-[1-(2-phenyl-ethanesulfonyl)-piperidin-4-yl]-methylamine and isonicotinic acid as described above in EXAMPLE 1, Step 2.

MS (m+1)=388.

### Example 175

N-{1-[2-(4-Fluoro-phenyl)-ethanesulfonyl]-piperidin-4-ylmethyl}-4-hydroxy-benzamide

Step 1:

1-(2-Chloro-ethyl)-4-fluoro-benzene

A mixture of 7 g of 2-(4-fluoro-phenyl)-ethanol, 25 mL of chlorobenzene, 42 mL of 37% HCl, and 0.9 g of Aliquat® 336 (tricaprylylmethyl ammonium chloride) was heated to reflux for 3 days, cooled and extracted into 3×100 mL of hexane. The combined extracts were dried over magnesium sulfate and concentrated under reduced pressure. The resulting oil was a crude product of 1-(2-chloro-ethyl)-4-fluoro-benzene:

¹H NMR (400 MHz, CDCl₃): 7.3 (dd, 211), 7.0 (dd, 2H), 3.7 (t, 2H), 3.05 (t, 2H).

Step 2:

Thioacetic acid S-[2.(4-fluoro-phenyl)-ethyl]ester

A mixture of 2.4 g of 1-(2-chloro-ethyl)-4-fluoro-benzene, 30 mL of DMF, and 2.5 g of potassium thioacetate was stirred under nitrogen for 24 h. The mixture was diluted with 200 mL of water and extracted with 3×50 mL of dichloromethane. The combined organic layers were dried over magnesium sulfate and concentrated under reduced pressure. Drying under vacuum gave the product as an oil:

¹H NMR (400 MHz, CDCl₃): 7.18 (dd, 2H), 6.98 (dd, 2H), 3.08 (t, 2H), 2.81 (t, 211), 2.32 (s, 3H).

Step 3:

2-(4-Fluoro-phenyl)-ethanesulfonyl chloride

A stream of chlorine gas was dispersed into a stirred, ice cold mixture of 2.5 g of thioacetic acid S-[2-(4-fluoro-phenyl)-ethyl]ester, 30 mL of dichloromethane and 30 mL of water over 1 h. The mixture was diluted with 200 mL of dichloromethane, shaken and separated. The combined organic layers were dried over magnesium sulfate and concentrated under reduced pressure. Trituration with hexane gave a white solid:

¹H NMR (400 MHz, CDCl₃): 7.2 (dd, 2H), 7.0 (dd, 2H), 3.1 (dd, 2H), 3.3 (dd, 2H), 2.32 (s, 3H).

Step 4:

4-(tert-Butoxycarbonylamino-methyl)-piperidine-1-carboxylic acid benzyl ester

To an ice cold, stirred solution of 21 g of 4-aminomethyl-piperidine-1-carboxylic acid benzyl ester in 250 m of dichloromethane was added 18 g of di-tert-butyldicarbonate in 100 mL of dichloromethane over 30 min. After stirring overnight, the mixture was concentrated to dryness. Trituration with hexane gave a white solid:

¹H NMR (400 MHz, CDCl₃): 7.4 (m, 5H), 5.15 (s, 2H), 4.6 (br s, 1H), 4.2 (br s, 2H), 3.0 (br s, 21), 2.8 ((m, 2H), 1.7 (m, 3H), 1.42 (s, 9H), 1.15 (m, 2H).

Step 5:

Piperidin-4-ylmethyl-carbamic acid tert-butyl ester

A mixture of 28 g of 4-(tert-butoxycarbonylamino-methyl)-piperidine-1-carboxylic acid benzyl ester, 1 g of 10% palladium on carbon, 100 mL of THF and 200 mL of methanol was stirred under anatmosphere of hydrogen for 2 days. The mixture was filtered concentrated under reduced pressure. Drying under reduced pressure gave a white solid:

¹H NMR (400 MHz, CDCl₃): 4.8 (br s, 1”), 3.05 (d, 2H), 2.9 (dd, 2H), 2.6 (m, 3H), 1.6 (d, 2H), 1.5 (m, 1H), 1.4 (s, 9H), 1.05 (m, 2H).

Step 6:

{1-[2-(4-Fluoro-phenyl)-ethanesulfonyl]-piperidin-4-ylmethyl}-carbamic acid tert-butyl ester

To an ice cold, stirred solution of 0.2 g of piperidin-4-ylmethyl-carbamic acid tert-butyl ester and 0.2 mL of N,N-diisopropylethyl amine in 20 mL of dichloromethane was added 0.3 g of 2-(4-fluoro-phenyl)-ethanesulfonyl chloride. After stirring overnight, the mixture was diluted with 50 mL of chloroform, washed with 50 mL of saturated sodium carbonate, dried over magnesium sulfate and concentrated to dryness under reduced pressure. Trituration with hexane gave a white solid:

¹H NMR (400 MHz, CDCl₃): 7.2 (m, 2H), 7.0 (dd, 2H), 4.6 (br m, 1H), 3.8 (d, 2H), 3.1 (m, 3H), 3.0 (m, 2H), 2.7 (dd, 2H), 1.8 (d, 2H), 1.6 (br m, 2H), 1.42 (s, 9H), 1.3 (m, 2H).

Step 7:

C-{1-[2-(4-Fluoro-phenyl)-ethanesulfonyl]-piperidin-4-yl}-methylamine

A mixture of 0.4 g of {1-[2-(4-fluoro-phenyl)-ethanesulfonyl]-piperidin-4-ylmethyl}-carbamic acid tert-butyl ester and 5 mL of 4N HCl in dioxane was stirred at rt for 3 h, then diluted with 50 mL of chloroform, washed with 50 mL of saturated sodium carbonate, dried over magnesium sulfate and concentrated to dryness under reduced pressure. The product was a white solid:

MS (m+1)=301;

¹H NMR (400 MHz, CDCl₃): 7.2 (m, 2H), 7.0 (dd, 2H), 3.92 (d, 2H), 3.1 (s, 4H), 2.7 (dd, 2H), 2.6 (d, 2H), 1.8 (d, 2H), 1.5 (br m, 3H), 1.3 (m, 2H).

Step 8

N-{1-[2-(4-Fluoro-phenyl)-ethanesulfonyl]-piperidin-4-ylmethyl}-4-hydroxy-benzamide

N-{1-[2-(4-Fluoro-phenyl)-ethanesulfonyl]-piperidin-4-ylmethyl}-4-hydroxy-benzamide was prepared from C-{1-[2-(4-fluoro-phenyl)-ethanesulfonyl]-piperidin-4-yl}-methylamine and 4-hydroxybenzoic acid as described above in EXAMPLE 1, Step 2.

MS (m+1)=421.

The following compounds were prepared as described in EXAMPLE 175, but replacing the 4-fluorophenethyl alcohol with the appropriately substituted phenethyl alcohol in Step 1 and using the appropriate carboxylic acid in Step 8.

| | | Analytical |
|---|---|---|
| EX. | Name | Data |
| 176 | N-[1-(2-p-Tolyl-ethanesulfonyl)- | MS (m + 1) = |
| | piperidin-4-ylmethyl]-isonicotinamide | 402.5. |
| 177 | 3H-Benzoimidazole-5-carboxylic acid[1-(2- | MS (m + 1) = |
| | phenyl-ethanesulfonyl)-piperidin- | 427.5. |
| | 4-ylmethyl]-amide | |
| 178 | Pyrimidine-4-carboxylic acid [1-(2-phenyl- | MS (m + 1) = 389. |
| | ethanesulfonyl)-piperidin-4-ylmethyl]-amide | |
| 179 | 2-Amino-pyrimidine-5-carboxylic acid[1-(2- | MS (m + 1) = 391 |
| | phenyl-ethanesulfonyl)-piperidin- | |
| | 4-ylmethyl]-amide | |
| 180 | Pyrazine-2-carboxylic acid[1-(2-phenyl- | MS (m + 1) = 389 |
| | ethanesulfonyl)-piperidin-4-ylmethyl]-amide | |
| 181 | 3-Amino-pyrazine-2-carboxylic acid[1- | MS (m + 1) = 404 |
| | (2-phenyl-ethanesulfonyl)-piperidin-4- | |
| | ylmethyl]-amide | |
| 182 | Pyrimidine-5-carboxylic acid[1-(2-phenyl- | MS (m + 1) = 389 |
| | ethanesulfonyl)-piperidin-4-ylmethyl]-amide | |
| 183 | Pyrimidine-4-carboxylic acid [1- | MS (m + 1) = 389 |
| | (2-p-tolyl-ethanesulfonyl)-piperidin-4- | |
| | ylmethyl]-amide | |
| 184 | 9H-Purine-6-carboxylic acid [1- | MS (m + 1) = 429 |
| | (2-phenyl-ethanesulfonyl)-piperidin-4- | |
| | ylmethyl]-amide | |
| 185 | N-{1-[2-(4-Chloro-phenyl)-ethanesulfonyl]- | MS (m + 1) = 437 |
| | piperidin-4-ylmethyl}-4-hydroxy-benzamide | |
| 186 | N-{1-[2-(2-Fluoro-phenyl)-ethanesulfonyl]- | MS (m + 1) = 421 |
| | piperidin-4-ylmethyl}-4-hydroxy-benzamide | |
| 187 | 6-Hydroxy-N-[1-(2-phenyl-ethanesulfonyl)- | MS (m + 1) = 404 |
| | piperidin-4-ylmethyl]-nicotinamide | |
| 188 | 4-Hydroxy-N-[1-(2-phenyl-ethanesulfonyl)- | MS (m + 1) = 403 |
| | piperidin-4-ylmethyl]-benzamide | |
| 189 | Pyridazine-4-carboxylic acid[1-(2-phenyl- | MS (m + 1) = 389 |
| | ethanesulfonyl)-piperidin-4-ylmethyl]-amide | |

### Example 190

(R,S) 3-[(4-Hydroxy-benzoylamino)-methyl]-pyrrolidine-1-carboxylic acid benzyl ester

Step 1:

1-Benzyl-pyrrolidine-3-carboxylic acid amide

To a mixture of 4.4 g of 1-benzyl-pyrrolidine-3-carboxylic acid methyl ester (M. J. Kornet et al., J. Org. Chem., 33:3637-3639(1968)) and 3 g of formamide in 10 mL of anhydrous DMF heated to 100° C. was added a solution of sodium methoxide, from 0.33 g of sodium dissolved in methanol, dropwise over 20 min. After stirring for 1 h at 100° C., the mixture was allowed to cool to rt and added to 100 mL of isopropanol. The mixture was concentrated to dryness. The resulting residue was triturated with 200 mL of chloroform, filtered and concentrated to dryness under reduced pressure. The resulting oil was fairly homogeneous by TLC (development with 90:10 chloroform saturated with ammonia: methanol):

¹H NMR (400 MHz, CDCl₃): 7.1 (5H), 4.3 (br s, 2H), 3.5 (d, 2H), 3.4 (m, 1H), 2.6 (m, 2H), 2.5 (m, 1H), 2.25 (m, 1H), 1.9 (m, 1H).

Step 2:

3-Carbamoyl-pyrrolidine-1-carboxylic acid benzyl ester

A mixture of 4.5 g of 1-benzyl-pyrrolidine-3-carboxylic acid amide, 200 mL of THF, 20 mL of methanol, and 1 g of 20% palladium hydroxide on carbon was shaken under 50 psi of hydrogen for 12 h. The catalyst was filtered off and the filtrate concentrated under reduced pressure. Drying under vacuum gave 3 g of an oil. To a stirred solution of the crude residue in 500 mL of chloroform was added 5.5 g of N-(benzyloxycarbonyloxy)succinimide and 2.2 mL of triethylamine. The mixture was allowed to stir overnight and washed with 50 mL of saturated sodium carbonate dried over magnesium sulfate and concentrated to dryness. Purification by chromatography on silica gel, eluting with 90:10 ethyl acetate: methanol, gave the product as a resin:

¹H NMR (400 MHz, CDCl₃): 7.35 (m, 5H), 5.6 (br m, 2H), 3.6 (m, 3H), 3.4 (m, 1H), 2.9 (br m, 1H), 2.1 (m, 2H).

Step 3:

3-Aminomethyl-pyrrolidine-1-carboxylic acid benzyl ester

A mixture of 1 g of 3-carbamoyl-pyrrolidine-1-carboxylic acid benzyl ester and 24 mL of 1 M borane-THF was stirred at room temperature for 24 h, then quenched with 50 mL of 3N HCl. The mixture was concentrated under reduced pressure, followed by being partitioned between 50 mL chloroform and 25 mL saturated aqueous sodium carbonate. Concentration of the combined extracts after drying over magnesium sulfate gave the product as a resin:

¹H NMR (400 MHz, CDCl₃)): 7.35 (m, 5H), 5.15 (s, 2H), 3.7-4 (complex, 4H), 2.7 (m, 1H), 2.4-2.0 (complex, 2H), 1.6 (m, 4H).

Step 4:

(R,S) 3-[(4-Hydroxy-benzoylamino)-methyl]-pyrrolidine-1-carboxylic acid benzyl ester

(R,S) 3-[(4-Hydroxy-benzoylamino)-methyl]-pyrrolidine-1-carboxylic acid benzyl ester was prepared from 3-aminomethyl-pyrrolidine-1-carboxylic acid benzyl ester and 4-hydroxybenzoic acid as described above in EXAMPLE 1, Step 2.

MS (m+1)=395.

### Example 191

(R) 3-[(4-Hydroxy-benzoylamino)-methyl]-pyrrolidine-1-carboxylic acid benzyl ester and (S) 3-[(4-Hydroxy-benzoylamino)-methyl]-pyrrolidine-1-carboxylic acid benzyl ester

Resolution of (R,S) 3-[(4-hydroxy-benzoylamino)-methyl]-pyrrolidine-1-carboxylic acid benzyl ester (EXAMPLE 190) was performed on a Chirapak® preparative chiral HPLC column:

MS (m+1)=395.

### Example 192

2-Amino-pyrimidine-5-carboxylic acid [1-(2-phenyl-ethanesulfonyl)-piperidin-4-ylmethyl]-amide

Step 1:

(5-{[1-(2-Phenyl-ethanesulfonyl)-piperidin-4-ylmethyl]-carbamoyl)-pyrimidin-2-yl)-carbamic acid tert-butyl ester (5-{[1-(2-Phenyl-ethanesulfonyl)-piperidin-4-ylmethyl]-carbamoyl}-pyrimidin-2-yl)-carbamic acid tert-butyl ester was prepared from C-[1-(2-phenyl-ethanesulfonyl)-piperidin-4-yl]-methylamine and 2-tert-butoxycarbonylamino-pyrimidine-5-carboxylic acid (prepared by BOC protection of ethyl 2-amino-5-pyrimidine carboxylate [prepared as described by P. Schenone, et al., J. Heterocyclic Chem., 27:295-305(1990)] using di-tert-butyl dicarbonate and 4-dimethylaminopyridine in acetonitrile, followed by saponification with sodium hydroxide and neutralization with dilute aqueous HCl) as described in EXAMPLE 1,

Step 2:

MS (m+1)=504.

Step 2:

2-Amino-pyrimidine-5-carboxylic acid [1-(2-phenyl-ethanesulfonyl)-piperidin-4-ylmethyl]-amide

2-Amino-pyrimidine-5-carboxylic acid [1-(2-phenyl-ethanesulfonyl)-piperidin-4-ylmethyl]-amide was prepared from (5-([1-(2-phenyl-ethanesulfonyl)-piperidin-4-ylmethyl]-carbamoyl}-pyrimidin-2-yl)-carbamic acid tert-butyl ester by stirring at rt for 3 h in 4N HCl in dioxane. The product was precipitated as the hydrochloride salt by dilution with ether and filtration.

MS (m+1)=404.

### Example 193

2-Amino-pyrimidine-5-carboxylic acid [1-(2-p-tolyl-ethanesulfonyl)-piperidin-4-ylmethyl]-amide

The title compound was prepared from C-[1-(2-p-tolyl-ethanesulfonyl)-piperidin-4-yl]-methylamine and 2-tert-butoxycarbonylamino-pyrimidine-5-carboxylic acid, followed by treatment with 4N HCl in dioxane as described in EXAMPLE 192.

MS (m+1)=418.

The following compounds were prepared by coupling 4-aminomethyl-piperidine-1-carboxylic acid benzyl ester (EXAMPLE 1, Step 1) with the appropriate acid as described in EXAMPLE 1, Step 2.

| EX. | Name | Analytical Data |
|---|---|---|
| 194 | 4-{[(3-Methyl-3H-imidazole-4- | MS (m + 1) = 357 |
| | carbonyl)-amino]-methyl}- | |
| | piperidine-1-carboxylic acid benzyl ester | |
| 195 | 4-{[(3-Methyl-3H-imidazole-4- | MS (m + 1) = 371 |
| | carbonyl)-amino]-methyl}-piperidine- | |
| | 1-carboxylic acid 4-methyl-benzyl ester | |
| 196 | 4-{[(9H-Purine-6-carbonyl)- | MS (m + 1) = 395 |
| | amino]-methyl}-piperidine-1- | |
| | carboxylic acid benzyl ester | |

### Example 197

3-Hydroxy-4[(4-hydroxy-benzoylamino)-methyl]-piperidine-1-carboxylic acid benzyl ester

Step 1:

1-Benzyl-4-hydroxymethyl-piperidin-3-ol

Sodium borohydride (40 g) was added in portions to a stirred solution of ethyl N-benzyl-3-oxopiperidine-4-carboxylate hydrochloride in methanol (500 mL), over 2 h. Water (300 mL) was added slowly, the mixture stirred for 15 min, and then the organics were evaporated. The resulting residue was partitioned between DCM and water (×3), the combined organic layers dried over anhydrous sodium sulfate, and the solvent evaporated to give the product as a cis trans mixture, used in the next step without further purification.

M.S. (M+1): 222.

Step 2:

3-Hydroxy-4-hydroxymethyl-piperidine-1-carboxylic acid benzyl ester

A solution of the 1-Benzyl-4-hydroxymethyl-piperidin-3-ol from Step 1 above (13.5 g) in methanol (450 mL) was hydrogenated at 50 psi over 20% palladium hydroxide on charcoal (10 g) for 48 h in three batches. The combined reaction mixtures were filtered and the filtrate evaporated to give an oil. This oil was dissolved in water (100 mL) and dioxane (100 mL), cooled to 5° C., and benzyl chloroformate (7.8 mL) was added slowly. 1M NaOH was added to maintain pH of 10-11. After 30 min, the cooling bath was removed and reaction mixture stirred for 30 min. The reaction mixture was concentrated to remove dioxane and the residue extracted with EtOAc (×3). The combined extracts were washed with brine, dried over anhydrous sodium sulfate and solvent evaporated to give a mixture of cis and trans products. Purified by flash column chromatography (80% EtOAc hexane to 5% MeOH EtOAc) gave the upper Rf cis isomer and the lower Rf trans isomer.

M.S. (M+1): 266.

Step 3:

Cis 3-Hydroxy-4-(toluene-4-sulfonyloxymethyl)-piperidine-1-carboxylic acid benzyl ester

A solution of the 3-Hydroxy-4-hydroxymethyl-piperidine-1-carboxylic acid benzyl ester diol from Step 2 above (7.65 g) in chloroform (200 mL) was treated with pyridine (2.6 mL) and 4-toluenesulfonyl chloride (6.05 g) and the reaction mixture heated to 60° C. for 18 h. Additional pyridine (0.85 mL) and 4-toluenesulfonyl chloride (2.0 g) were added to the cooled reaction and heating continued for a further 24 h. The reaction mixture was cooled to rt and washed with 10% aqueous citric acid solution and water, dried over anhydrous sodium sulfate and the solvent evaporated to give, after flash column chromatography, the Cis 3-Hydroxy-4-(toluene-4-sulfonyloxymethyl)-piperidine-1-carboxylic acid benzyl ester.

Step 4:

Cis 4-Aminomethyl-3-hydroxy-piperidine-1-carboxylic acid benzyl ester

A solution of the cis 3-Hydroxy-4-(toluene-4-sulfonyloxymethyl)-piperidine-1-carboxylic acid benzyl ester (6.80 g) from Step 3 above was dissolved in DMF (5 mL) and treated with sodium azide (3.16 g). The reaction mixture was then heated to 50° C. for 48 h, cooled to rt, and partitioned between dilute aqueous sodium bicarbonate and EtOAc. The organic layer was washed with brine, dried over anhydrous sodium sulfate and solvent evaporated to give the azide, which was dissolved in T]THF (50 mL) and treated with triphenylphosphine (14.07 g) and water (3.25 mL). The reaction mixture was stirred for 18 h at rt, the volatiles evaporated, and the residue purified by flash column chromatography (DCM to 80/20/2 DCM MeOH NH₄OH) to give the cis 4-Aminomethyl-3-hydroxy-piperidine-1-carboxylic acid benzyl ester as an oil.

M.S. (M+1): 265.

Step 4:

3-Hydroxy-4-[(4-hydroxy-benzoylamino)-methyl]-piperidine-1-carboxylic acid benzyl ester

The 3-Hydroxy-4-[(4-hydroxy-benzoylamino)-methyl]-piperidine-1-carboxylic acid benzyl ester was prepared from the cis 4-Aminomethyl-3-hydroxy-piperidine-1-carboxylic acid benzyl ester (Step 3 above) and 4-hydroxybenzoic acid as described in EXAMPLE 1, Step 2.

### Example 198

3-[(4-Hydroxy-benzoylamino)-methyl]-piperidine-1-carboxylic acid benzyl ester

Step 1:

4-Hydroxy-N-pyridin-3-ylmethyl-benzamide

The 4-hydroxy-N-pyridin-3-ylmethyl-benzamide was prepared from 3-(2-aminomethyl)pyridine and 4-hydroxybenzoic acid in as described in EXAMPLE 1, Step 2.

M.S. (M+1): 229.

Step 2:

4-Hydroxy-N-piperidin-3-ylmethyl-benzamide

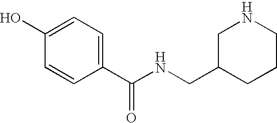

To a solution of 4-hydroxy-N-pyridin-3-ylmethyl-benzamide (2.0 g, 0.0088 mol) in acetic acid (135 mL) was added platinum oxide (200 mg) and the mixture stirred under hydrogen for 3 h. The reaction was filtered and concentrated in vacuo to give an oil.

M.S. (M+1): 235.

Step 3:

3-[(4-Hydroxy-benzoylamino)-methyl]-piperidine-1-carboxylic acid benzyl ester

To a mixture of 4-hydroxy-N-piperidin-3-ylmethyl-benzamide (135 mg, 0.580 mmol) in tetrahydrofuran (5 mL) was added triethylamine (100 μL) and N-benzyloxycarbonyloxysuccinamide (144 mg, 0.580 mmol) and the mixture stirred at rt for 3 h. The reaction was concentrated in vacuo and chromatographed on silica using 50-100% ethyl acetate/hexane to give 3-[(4-hydroxy-benzoylamino)-methyl]-piperidine-1-carboxylic acid benzyl ester as a foam.

M.S. (M+1): 369.

### Example 199

3-[(4-Hydroxy-benzoylamino)-methyl]-piperazine-1-carboxylic acid benzyl ester

Step 1:

1,4-Dibenzyl-2-chloromethyl-piperazin

The above compound was prepared according to the procedure described in Bihan, G. et. al., J. Med. Chem., 42:1587-1603(1999).

Step 2:

2-Azidomethyl-1,4-dibenzyl-piperazine

To a solution of 1,4-dibenzyl-2-chloromethyl-piperazine (8.8 g, 0.028 mol) in dimethylformamide (90 mL) under nitrogen was added sodium azide (5.5 g) and the reaction stirred at 50° C. for 18 h. The reaction was cooled and diluted with 10% aqueous sodium bicarbonate (100 mL) and water (250 mL) and the mixture extracted with ethyl acetate (2×200 mL). The organic extracts were washed with 10% sodium bicarbonate, brine, dried over sodium sulfate and concentrated to an oil.

M.S. (M+1): 322.

Step 3:

C-(1,4-Dibenzyl-piperazin-2-yl)-methylamine

To a solution of 2-azidomethyl-1,4-dibenzyl-piperazine (9.0 g, 0.028 mol) in THF (90 mL) and water (5 mL) was added triphenylphosphine (22.3 g, 0.085 mol) and the mixture stirred for 18 h. The reaction was concentrated to an oil, dissolved in 1N hydrochloric acid (100 mL) and washed with ethyl acetate (2×100 mL). The acidic aqueous layer was cooled to 0° C. and the pH adjusted to 8.5 with 3N sodium hydroxide. The mixture was extracted with ethyl acetate (2×100 mL) and extracts dried over sodium sulfate and concentrated to an oil.

M.S. (M+1): 296.

Step 4:

N-(1,4-Dibenzyl-piperazin-2-ylmethyl)-4-hydroxy-benzamide

The N-(1,4-Dibenzyl-piperazin-2-ylmethyl)4-hydroxy-benzamide was prepared from C-(1,4-Dibenzyl-piperazin-2-yl)-methylamine and 4-hydroxybenzoic acid as described in EXAMPLE 1, Step 2.

M.S. (M+1): 416.

Step 5:

4-Hydroxy-N-piperazin-2-ylmethyl-benzamide

The 4-Hydroxy-N-piperazin-2-ylmethyl-benzamide was prepared according to the procedure described in EXAMPLE 198, Step 2, using 10% Palladium/Carbon as catalyst in ethanol/12N HCl at 50° C. for 5 h.

M.S. (M+1): 236.

Step 6:

3-[(4-Hydroxy-benzoylamino)-methyl]-piperazine-1-carboxylic acid benzyl ester

The 3-[(4-Hydroxy-benzoylamino)-methyl]-piperazine-1-carboxylic acid benzyl ester was prepared according to the procedure described in EXAMPLE 198, Step 3. Dilution of reaction with 10% aqueous sodium bicarbonate and extraction with ethyl acetate followed by concentration and purification by silica gel chromatography using 95/5/1 to 90/10/2 (dichloromethane/methanol/NH₄OH) gave the 3-[(4-Hydroxy-benzoylamino)-methyl]-piperazine-1-carboxylic acid benzyl ester as a solid.

M.S. (+1): 370.

### Example 200

4-Hydroxy-N-[4-(3-phenyl-propionyl)-piperazin-2-ylmethyl]-benzamide

The title compound was prepared in a similar manner as described in EXAMPLE 1, Step 2, from 4-hydroxy-N-piperazin-2-ylmethyl-benzamide and 4-hydroxybenzoic acid.

M.S. (M+1): 368.

### Example 201

4-Hydroxy-N-[4-(3-phenyl-propyl)-piperazin-2-ylmethyl]-benzamide

The title compound was prepared in a similar manner as described in EXAMPLE 148, Step 1, from 4-Hydroxy-N-piperazin-2-ylmethyl-benzamide and propionaldehyde in dichlorethane as solvent.

M.S. (M+1): 354.

### Example 202

2-[(4-Hydroxy-benzoylamino)-methyl]-morpholine-4-carboxylic acid benzyl ester

Step 1:

N-(4-Benzyl-morpholin-2-ylmethyl)-4-hydroxy-benzamide

The N-(4-Benzyl-morpholin-2-ylmethyl)₄-hydroxy-benzamide was prepared from C-(4-benzyl-morpholin-2-yl)-methylamine (S. Kato et al., J. Med Chem., 33:1406(1990)) similarly to the procedure described in EXAMPLE 1, Step 2.

M.S. (M+1): 327

Step 2:

A solution of N-(4-benzyl-morpholin-2-ylmethyl)₄-hydroxy-benzamide (Step 1 above) (320 mg) was dissolved in ethanol (20 mL) and hydrogenated at 1 atm over 20% Pd(OH)₂/C (250 mg) for 18 h. The catalyst was removed by filtration, washed with ethanol, and the filtrate evaporated, to give a solid. A portion (21 mg) of this material was dissolved in DMF (0.5 mL) and N-(benzyloxycarbonyloxy)succinimide (27 mg) was added. The reaction mixture was stirred for 10 min, one drop of water was added and the solution was purified by preparative reverse phase HPLC to give the 2-[(4-Hydroxy-benzoylamino)-methyl]-morpholine-4-carboxylic acid benzyl ester compound.

M.S. (M+1): 371

### Example 203

4-Hydroxy-N-[4-(3-phenyl-propyl)-morpholin-2-ylmethyl]-benzamide

A solution of N-(4-benzyl-morpholin-2-ylmethyl)4-hydroxy-benzamide (EXAMPLE 202, Step 1) (55 mg) was dissolved in acetic acid (3 mL) and hydrogenated at 1 atm over 10% Pd/C (50 mg) for 18 h. The catalyst was removed by filtration, washed with acetic acid and the filtrate evaporated, to give an oil. A portion of this oil (21 mg) was dissolved in methanol (1 mL) and treated with phenylpropionaldehyde (24 mg) and sodium cyanoborohydride (25 mg). The resulting reaction was stirred for 15 min and the crude reaction mixture purified by preparative reverse phase HPLC to give the 4-Hydroxy-N-[4-(3-phenyl-propyl)-morpholin-2-ylmethyl]-benzamide compound.

M.S. (M+1): 355

What is claimed is:

1. A method for treating or preventing migraines in a mammalian patient in need of such treatment or prevention comprising administering to said patient an NR₂B receptor antagonist in an amount that is effective to treat or prevent migraines.

2. The method according to claim 1 wherein the NR2B antagonist is administered at a dose ranging from about 0.1 mg to about 2500 mg.

3. The method according to claim 1 wherein the mammalian patient is human.

4. The method for treating migraines in a mammalian patient in need of such treatment comprising administering to said patient an NR2B receptor antagonist in an amount that is effective to treat migraines in accordance with claim 1.

5. The method for preventing migraines in a mammalian patient in need of such prevention comprising administering to said patient an NR2B antagonist in an amount that is effective to prevent migraines in accordance with claim 1.

6. The method according to claim 1 further comprising concomitantly administering a calcitonin gene-related peptide receptor (CGRP) ligand with said NR2B receptor antagonist in amounts that are effective to treat or prevent migraines.

7. The method according to claim 1 further comprising concomitantly administering a cyclooxygenase-2 selective inhibiting compound with said NR2B receptor antagonist in amounts that are effective to treat or prevent migraines.

8. The method according to claim 7 wherein the cyclooxygenase-2 selective inhibiting compound is selected from the group consisting of: celecoxib, rofecoxib, etoricoxib, valdecoxib, parecoxib, COX189, BMS347070, ABT963, CS502, GW406381 and JTE522.

9. The method according to claim 8 wherein the cyclooxygenase-2 selective inhibiting compound is rofecoxib.

10. The method according to claim 8 wherein the cyclooxygenase-2 selective inhibiting compound is etoricoxib.

11. The method according to claim 1 further comprising concomitantly administering a 5HT_{1B/1D} agonist with said NR2B receptor antagonist in amounts that are effective to treat or prevent migraines.

12. The method according to claim 11 wherein the 5HT_{1B/1D} agonist is selected from the group consisting of: rizatriptan, sumatriptan, naratriptan, zolmitriptan, eleptriptan, and almotriptan

13. The method according to claim 12 wherein the 5HT_{1B/1D} agonist is rizatriptan.

14. The method according to claim 1 further comprising concomitantly administering a leukotriene receptor antagonist with said NR2B receptor antagonist in amounts that are effective to treat or prevent migraines.

15. The method according to claim 14 wherein the leukotriene receptor antagonist is montelukast.

16. A pharmaceutical composition comprising an NR2B receptor antagonist and a calcitonin gene-related peptide receptor (CGRP) receptor ligand in combination with a pharmaceutically acceptable carrier.

17. A pharmaceutical composition comprising an NR2B receptor antagonist and a 5HT_{1B/D} agonist in combination with a pharmaceutically acceptable carrier.

18. The pharmaceutical composition according to claim 17 wherein the 5HT_{1B/1D} agonist is rizatriptan.

19. A pharmaceutical composition comprising an NR2B receptor antagonist and a leukotriene receptor antagonist in combination with a pharmaceutically acceptable carrier.

20. The pharmaceutical composition according to claim 19 wherein the leukotriene receptor antagonost is montelukast.
